(19) United States
(12) Patent Application Publication
(10) Pub. No.: US 2005/0096360 A1
(43) Pub. Date: May 5, 2005

(54) Inhibitors of semicarbazide-sensitive amine oxidase (SSAO) and VAP-1 mediated adhesion useful for treatment of diseases

(76) Inventors: Luisa Maria Salter-Cid, San Diego, CA (US); Eric Yanjun Wang, San Diego, CA (US); Keith Cockerill, San Diego, CA (US); Matthew D. Linnik, Solana Beach, CA (US); Edward J. Victoria, San Diego, CA (US)

Correspondence Address: MORRISON & FOERSTER LLP, 755 PAGE MILL RD, PALO ALTO, CA, 94304-1018, US

(21) Appl. No.: 10/913,253

(22) Filed: Aug. 6, 2004

Related U.S. Application Data
(60) Provisional application No. 60/493,835, filed on Aug. 8, 2003.
(60) Provisional application No. 60/502,401, filed on Sep. 12, 2003.
(60) Provisional application No. 60/568,999, filed on May 6, 2004.

(51) Int. Cl. A61K 31/44; A61K 31/15

(52) U.S. Cl. 514/357; 514/645; 514/646; 546/332; 564/300; 564/310

(57) ABSTRACT

Compositions and methods of using compositions for treatment of inflammatory diseases and immune disorders are provided. Allylhydrazine compounds, hydroxylamine (aminooxy) compounds, and other compounds are disclosed which are inhibitors of semicarbazide-sensitive amine oxidase (SSAO) and/or vascular adhesion protein 1 (VAP-1). The compounds have therapeutic utility in suppressing inflammation and inflammatory responses, and in treatment of several disorders, including multiple sclerosis.

### CROSS REFERENCE TO RELATED APPLICATIONS

This application claims priority benefit of U.S. Provisional Patent Application No. 60/493,835, filed Aug. 8, 2003, of U.S. Provisional Patent Application No. 60/502,401, filed Sep. 12, 2003, and of U.S. Provisional Patent Application No. 60/568,999 filed May 6, 2004. The entire contents of those applications are hereby incorporated by reference herein.

### STATEMENT REGARDING FEDERALLY SPONSORED RESEARCH OR DEVELOPMENT

Not applicable.

### TECHNICAL FIELD

This application relates to compositions and methods for inhibiting semicarbazide-sensitive amine oxidase (SSAO), also known as vascular adhesion protein-1 (VAP-1), for treatment of inflammation, inflammatory diseases and autoimmune disorders.

### BACKGROUND

Human vascular adhesion protein-I (VAP-1) is a type 2, 180 kD homodimeric endothelial cell adhesion molecule. Cloning and sequencing of VAP-1 revealed that the VAP-1 cDNA sequence is identical to that of the previously known protein semicarbazide-sensitive amine oxidase (SSAO), a copper-containing amine oxidase. The precise difference (if any) between the membrane-bound VAP-1 adhesion protein and the soluble SSAO enzyme has not yet been determined; one hypothesis indicates that proteolytic cleavage of the membrane-bound VAP-1 molecule results in the soluble SSAO enzyme. Both the membrane-bound VAP-1 protein and the soluble SSAO enzyme have amine oxidase enzymatic activity. Thus membrane-bound VAP-1 can function both as an amine oxidase and a cell adhesion molecule.

Semicarbazide-sensitive amine oxidase is a member of a group of enzymes; that group is referred to generically as semicarbazide-sensitive amine oxidases (SSAOs). SSAOs are mostly soluble enzymes that catalyze oxidative deamination of primary amines. The reaction results in the formation of the corresponding aldehyde and release of H₂O₂ and ammonium. These enzymes are different from monoamine oxidases A and B (MAO-A and MAO-B, respectively), in terms of their substrates, inhibitors, cofactors, subcellular localization and function. To date, no physiological function has been definitively associated with SSAOs, and even the nature of the physiological substrates is not firmly established (reviewed in Buffoni F. and Ignesti G. (2000) Mol. Genetics Metabl. 71:559-564). However, they have been implicated in the metabolism of exogenous and endogenous amines and in the regulation of glucose transport.

SSAO molecules are highly conserved across species; the closest homologue to the human protein is the bovine serum amine oxidase (about 85% identity). Substrate specificity and tissue distribution vary considerably among different species. In humans, SSAO specific activity has been detected in most tissues but with marked differences (highest in aorta and lung). Human and rodent plasma have very low SSAO activity compared with ruminants. Depletion studies suggest that SSAO/VAP-1 accounts for ˜90% of cell and serum SSAO activity (Jaakkola K. et al.(1999) Am. J. Pathol. 155:1953).

Membrane-bound VAP-1 is primarily expressed in high endothelial cells (ECs) of lymphatic organs, sinusoidal ECs of the liver and small caliber venules of many other tissues. Moreover, SSAO/VAP-1 is also found in dendritic cells of germinal centers and is abundantly present in adipocytes, pericytes and smooth muscle cells. However, it is absent from capillaries, ECs of large blood vessels, epithelial cells, fibroblasts and leukocytes (Salmi M. et al. (2001) Trends Immunol. 22:211). Studies in clinical samples revealed that SSAO/VAP-1 is upregulated on vasculature at many sites of inflammation, such as synovitis, allergic and other skin inflammations, and inflammatory bowel disease (IBD). However, expression appears to be controlled by additional mechanisms. Animal studies indicate that the luminal SSAO/VAP-1 is induced only upon elicitation of inflammation. Thus, in ECs, SSAO/VAP-1 is stored in intracellular granules and is translocated onto the luminal surface only at sites of inflammation.

In the serum of healthy adults a soluble form of SSAO/VAP-1 is found at a concentration of 80 ng/ml. Soluble SSAO/VAP-1 levels increase in certain liver diseases and in diabetes, but remain normal in many other inflammatory conditions. Soluble SSAO/VAP-1 has an N-terminal amino acid sequence identical to the proximal extracellular sequence of the membrane bound form of SSAO/VAP-1. In addition, there is good evidence that at least a significant portion of the soluble molecule is produced in the liver by proteolytic cleavage of sinusoidal VAP-1 (Kurkijarvi R. et al. (2000) Gastroenterology 119:1096).

SSAO/VAP-1 regulates leukocyte-subtype-specific adhesion to ECs. Studies show that SSAO/VAP-1 is involved in the adhesion cascade at sites where induction/activation of selectins, chemokines, immunoglobulin superfamily molecules, and integrins takes place. In the appropriate context, nevertheless, inactivation of SSAO/VAP-1 function has an independent and significant effect on the overall extravasion process. A recent study shows that both the direct adhesive and enzymatic functions of SSAO/VAP-1 are involved in the adhesion cascade (Salmi M. et al. (2001) Immunity 14:265). In this study, it was proposed that the SSAO activity of VAP-1 is directly involved in the pathway of leukocyte adhesion to endothelial cells by a novel mechanism involving direct interaction with an amine substrate presented on a VAP-1 ligand expressed on the surface of a leukocyte. Under physiological laminar shear, it seems that SSAO/VAP-1 first comes into play after tethering (which takes place via binding of selectins to their ligands) when lymphocytes start to roll on ECs. Accordingly, anti-VAP-1 monoclonal antibodies inhibit ˜50% of lymphocyte rolling and significantly reduce the number of firmly bound cells. In addition, inhibition of VAP-1 enzymatic activity by SSAO inhibitors, also results in a >40% reduction in the number of rolling and firmly bound lymphocytes. Thus, inhibitors of SSAO/VAP-1 enzymatic activity could reduce leukocyte adhesion in areas of inflammation and thereby reduce leukocyte trafficking into the inflamed region and, consequently, reduce the inflammatory process itself.

Increased SSAO activity has been found in the plasma and islets of Type I and Type II diabetes patients and animal models, as well as after congestive heart failure, and in an artherosclerosis mouse model (Salmi M,.et al. (2002) Am. J. Pathol. 161:2255; Bono P. et al (1999) Am. J. Pathol. 155:1613; Boomsma F. et al (1999) Diabetologia 42:233; Gronvall-Nordquist J. et al (2001) J. Diabetes Complications 15:250; Ferre I. et al. (2002) Neurosci. Lett. 15; 321: 21; Conklin D. J. et al. (1998) Toxicological Sciences 46: 386; Yu P. H. and Deng Y. L. (1998) Atherosclerosis 140:357; Vidrio H. et al. (2002) General Pharmacology 35:195; Conklin D. J. (1999) Toxicology 138: 137). In addition to upregulation of expression of VAP-1 in the inflamed joints of rheumatoid arthritis (RA) patients and in the venules from lamina propria and Peyer's patches of IBD patients, increased synthesis of VAP-1 was also found in chronic skin inflammation and liver disease (Lalor P. F. et al. (2002) J. Immunol. 169:983; Jaakkola K. et al. (2000) Am. J. Pathol. 157:463; Salmi M. and Jalkanen S. (2001) J. Immunol. 166:4650; Lalr P. F. et al. (2002) Immunol Cell Biol 80:52; Salmi M et al. (1997) J. Cin. Invest. 99:2165; Kurkijarvi R. et al. (1998) J. Immunol. 1611549).

In summary, SSAO/VAP-1 is an inducible endothelial enzyme that regulates leukocyte-subtype-specific adhesion and mediates the interaction between lymphocytes and inflamed vessels. The fact that SSAO/VAP-1 has both enzymatic and adhesion activities together with the strong correlation between its upregulation in many inflammatory conditions, makes it a potential therapeutic target for all the above-mentioned disease conditions.

### DISCLOSURE OF THE INVENTION

SSAO inhibitors can block inflammation and autoimmune processes, as well as other pathological conditions associated with an increased level of the circulating amine substrates and/or products of SSAO. In one embodiment, the invention relates to a method of inhibiting an inflammatory response by administration of compounds to inhibit SSAO enzyme activity (where the enzyme activity is due either to soluble SSAO enzyme or membrane-bound VAP-1 protein, or due to both) and/or inhibit binding to VAP-1 protein. In another embodiment, the inflammatory response is an acute inflammatory response. In another embodiment, the invention relates to treating diseases mediated at least in part by SSAO or VAP-1, as generally indicated by one or more of abnormal levels of SSAO and/or VAP-1 or abnormal activity of SSAO and/or VAP-1 (where the abnormal activity of VAP-1 may affect its binding function, its amine oxidase function, or both), by administering a therapeutically effective amount of an SSAO inhibitor, or administering a therapeutically effective combination of SSAO inhibitors. In another embodiment, the invention relates to a method of treating immune disorders, by administering a therapeutically effective amount of an SSAO inhibitor, or administering a therapeutically effective combination of SSAO inhibitors. In another embodiment, the invention relates to a method of treating multiple sclerosis (including chronic multiple sclerosis), by administering a therapeutically effective amount of an SSAO inhibitor, or administering a therapeutically effective combination of SSAO inhibitors. In another embodiment, the invention relates to a method of treating ischemic diseases (for example, stroke) and/or the sequelae thereof (for example, an inflammatory response), by administering a therapeutically effective amount of an SSAO inhibitor, or administering a therapeutically effective combination of SSAO inhibitors. The SSAO inhibitors administered can inhibit the SSAO activity of soluble SSAO, the SSAO activity of membrane-bound VAP-1, binding to membrane-bound VAP-1, or any two of those activities, or all three of those activities. In another embodiment, the invention relates to a method of inhibiting SSAO activity or inhibiting binding to VAP-1 in vitro using the compounds provided herein. In another embodiment, the invention relates to a method of inhibiting SSAO activity or inhibiting binding to VAP-1 in vivo, that is, in a living organism, such as a vertebrate, mammal, or human, using the compounds provided herein.

In another embodiment, the present invention relates to various compounds which are useful for inhibiting SSAO enzyme activity (where the enzyme activity is due either to soluble SSAO enzyme or membrane-bound VAP-1 protein, or due to both) and/or inhibition of binding to membrane-bound VAP-1 protein. In another embodiment, the present invention relates to methods of using various compounds to inhibit SSAO enzyme activity (where the enzyme activity is due either to soluble SSAO enzyme or membrane-bound VAP-1 protein, or due to both) and/or inhibit binding to VAP-1 protein.

In another embodiment, the present invention relates to methods of treating inflammation, by administering an SSAO inhibitor which has a specificity for inhibition of SSAO as compared to MAO-A and/or MAO-B of about 10, about 100, or about 500.

In another embodiment, the present invention relates to methods of treating an immune or autoimmune disorder, by administering an SSAO inhibitor which has a specificity for inhibition of SSAO as compared to MAO-A and/or MAO-B of about 10, about 100, or about 500.

In another embodiment, the present invention relates to methods of treating inflammation, by administering one or more of the compounds described herein in formulas I, I-P, I-A, I-AP, I-B, I-C, II, III, III-A, III-B, or III-C in a therapeutically effective amount, or in an amount sufficient to treat inflammation. In another embodiment, the present invention relates to methods of treating immune or autoimmune disorders, by administering one or more of the compounds described herein in formulas I, I-P, I-A, I-AP, I-B, I-C, II, III, III-A, III-B, or III-C in a therapeutically effective amount, or in an amount sufficient to treat an immune or autoimmune disorder.

In one embodiment, the present invention relates to compounds of formula I: 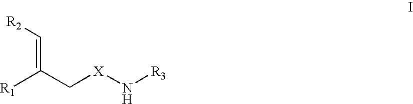
wherein:
R₁ is independently chosen from the group consisting of H, C₁-C₄ alkyl,
C₃-C₈ cycloalkyl, C₆-C₁₀ aryl, C₆-C₁₄ aralkyl, C₄-C₉ heteroaryl, C₆-C₁₄ substituted aryl,
C₅-C₁₄ substituted heteroaryl, R₄—(CH₂)ₙ—, and R₅—Y₁—CH₂—;
n is independently 1 or 2;
Y₁ is independently S or O;
R₂ is independently chosen from H, C₁-C₄ alkyl, Cl, F, or CF₃;
X is independently chosen from O or NR₆;
R₃ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₄ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₅ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl; and
R₆ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
   including all stereoisomers thereof, all E/Z (cis/trans) isomers thereof, all solvates and hydrates thereof, all crystalline and non-crystalline forms thereof, and all salts thereof, particularly pharmaceutically-acceptable salts. Metabolites and prodrugs of the compounds of formula I are also embraced by the invention. In one preferred embodiment, R₁ is unsubstituted phenyl. In another preferred embodiment, R₁ is substituted phenyl. In another preferred embodiment, R₁ is substituted phenyl bearing one substituent. In another preferred embodiment, R₁ is substituted phenyl bearing two substituents. In another preferred embodiment, R₂ is H. In another preferred embodiment, R₂ is F. In another embodiment, X is O. In another preferred embodiment, X is NR₆. In another preferred embodiment, R₃ is H or C₁-C₄ alkyl. In another preferred embodiment, R₆ is H or C₁-C₄ alkyl.

In another embodiment, the present invention relates to methods of using the compounds of formula I to inhibit SSAO enzyme activity (whether the enzyme activity is due either to soluble SSAO enzyme or membrane-bound VAP-1 protein, or due to both) and/or inhibit binding to VAP-1 protein. The compounds can be used for a method of inhibiting SSAO activity or inhibiting binding to VAP-1 in vitro, by supplying the compound to the in vitro environment in an amount sufficient to inhibit SSAO activity or inhibit binding to VAP-1. The compounds can also be used for a method of inhibiting SSAO activity or inhibiting binding to VAP-1 in vivo, that is, in a living organism, such as a vertebrate, mammal, or human, by administering the compounds to the organism in an amount sufficient to inhibit SSAO activity or inhibit binding to VAP-1. In another embodiment, the present invention relates to methods of using the compounds of formula I to treat inflammation or immune disorders. In another embodiment, the present invention relates to methods of using the compounds of formula I to suppress or reduce inflammation, or to suppress or reduce an inflammatory response. In another embodiment, the present invention relates to methods of treating inflammation, by administering one or more of the compounds described in formula I in a therapeutically effective amount, or in an amount sufficient to treat inflammation. In another embodiment, the present invention relates to methods of treating immune or autoimmune disorders, by administering one or more of the compounds described in formula I in a therapeutically effective amount, or in an amount sufficient to treat the immune or autoimmune disorder.

In another embodiment, the present invention relates to compounds of formula I-P: 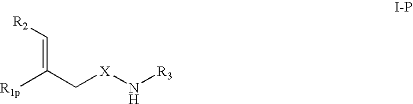
wherein:
R₁ₚ is independently chosen from the group consisting of H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl, C₅-C₁₄ substituted heteroaryl, R₄—(CH₂)ₙ—, and R₅—Y₁—CH₂—;
n is independently 1 or 2;
Y₁ is independently S or O;
R₂ is independently chosen from H, C₁-C₄ alkyl, Cl, F, or CF₃;
X is independently chosen from O or NR₆;
R₃ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₄ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₅ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl; and
R₆ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
   with the proviso that when R₁ₚ is unsubstituted phenyl, R₂ is H, and X is NH, then R₃ is not H;
   including all stereoisomers thereof, all E/Z (cis/trans) isomers thereof, all solvates and hydrates thereof, all crystalline and non-crystalline forms thereof, and all salts thereof, particularly pharmaceutically-acceptable salts. This subset of compounds of formula I, inclusive of the proviso set forth above, are designated the compounds of subset P of formula I, or the compounds of formula I-P. Metabolites and prodrugs of the compounds of formula I are also embraced by the invention. In one preferred embodiment, R₁ₚ is unsubstituted phenyl. In another preferred embodiment, R₁ₚ is substituted phenyl. In another preferred embodiment, R₁ₚ is substituted phenyl bearing one substituent. In another preferred embodiment, R₁ₚ is substituted phenyl bearing two substituents. In another preferred embodiment, R₂ is H. In another embodiment, X is O. In another preferred embodiment, X is NR₆. In another preferred embodiment, R₃ is H or C₁-C₄ alkyl. In another preferred embodiment, R₆ is H or C₁-C₄ alkyl.

In another embodiment, the present invention relates to methods of using the compounds of formula I-P to inhibit SSAO enzyme activity (whether the enzyme activity is due either to soluble SSAO enzyme or membrane-bound VAP-1 protein, or due to both) and/or inhibit binding to VAP-1 protein. The compounds can be used for a method of inhibiting SSAO activity or inhibiting binding to VAP-1 in vitro, by supplying the compound to the in vitro environment in an amount sufficient to inhibit SSAO activity or inhibit binding to VAP-1. The compounds can also be used for a method of inhibiting SSAO activity or inhibiting binding to VAP-1 in vivo, that is, in a living organism, such as a vertebrate, mammal, or human, by administering the compounds to the organism in an amount sufficient to inhibit SSAO activity or inhibit binding to VAP-1. In another embodiment, the present invention relates to methods of using the compounds of formula I-P to treat inflammation or immune disorders. In another embodiment, the present invention relates to methods of using the compounds of formula I-P to suppress or reduce inflammation, or to suppress or reduce an inflammatory response. In another embodiment, the present invention relates to methods of treating inflammation, by administering one or more of the compounds described in formula I-P in a therapeutically effective amount, or in an amount sufficient to treat inflammation. In another embodiment, the present invention relates to methods of treating immune or autoimmune disorders, by administering one or more of the compounds described in formula I-P in a therapeutically effective amount, or in an amount sufficient to treat the immune or autoimmune disorder.

In one embodiment, the present invention relates to compounds of formula I-A: 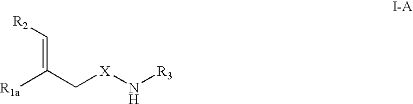
wherein:
R₁ₐ is substituted or unsubstituted phenyl;
R₂ is independently chosen from H, C₁-C₄ alkyl, Cl, F, or CF₃;
X is independently chosen from O or NR₆;
R₃ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₆ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
   including all stereoisomers thereof, all E/Z (cis/trans) isomers thereof, all solvates and hydrates thereof, all crystalline and non-crystalline forms thereof, and all salts thereof, particularly pharmaceutically-acceptable salts. This subset of compounds of formula I are designated the compounds of subset A of formula I, or the compounds of formula I-A. Metabolites and prodrugs of the compounds of formula I-A are also embraced by the invention. In one embodiment, R₁ₐ is unsubstituted phenyl. In another embodiment, R₁ₐ is substituted phenyl. In another embodiment, R₁ₐ is substituted phenyl bearing one substituent. In another embodiment, R₁ₐ is substituted phenyl bearing two substituents. In another embodiment, X is O. In another embodiment, X is NR₆. In another embodiment, R₃ is H or C₁-C₄ alkyl. In another embodiment, R₆ is H or C₁-C₄ alkyl.

In another embodiment, the present invention relates to methods of using the compounds of formula I-A to inhibit SSAO enzyme activity (whether the enzyme activity is due either to soluble SSAO enzyme or membrane-bound VAP-1 protein, or due to both) and/or inhibit binding to VAP-1 protein. The compounds can be used for a method of inhibiting SSAO activity or inhibiting binding to VAP-1 in vitro, by supplying the compound to the in vitro environment in an amount sufficient to inhibit SSAO activity or inhibit binding to VAP-1. The compounds can also be used for a method of inhibiting SSAO activity or inhibiting binding to VAP-1 in vivo, that is, in a living organism, such as a vertebrate, mammal, or human, by administering the compounds to the organism in an amount sufficient to inhibit SSAO activity or inhibit binding to VAP-1. In another embodiment, the present invention relates to methods of using the compounds of formula I-A to treat inflammation or immune disorders. In another embodiment, the present invention relates to methods of using the compounds of formula I-A to suppress or reduce inflammation, or to suppress or reduce an inflammatory response. In another embodiment, the present invention relates to methods of treating inflammation, by administering one or more of the compounds described in formula I-A in a therapeutically effective amount, or in an amount sufficient to treat inflammation. In another embodiment, the present invention relates to methods of treating immune or autoimmune disorders, by administering one or more of the compounds described in formula I-A in a therapeutically effective amount, or in an amount sufficient to treat the immune or autoimmune disorder.

In another embodiment, the present invention relates to compounds of formula I-AP: 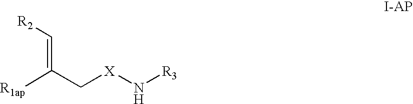
wherein:
R₁ₐₚ is substituted or unsubstituted phenyl;
R₂ is independently chosen from H, C₁-C₄ alkyl, Cl, F, or CF₃;
X is independently chosen from O or NR₆;
R₃ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₆ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
   with the proviso that when R₁ₐₚ is unsubstituted phenyl, R₂ is H, and X is NH, then R₃ is not H;
   including all stereoisomers thereof, all E/Z (cis/trans) isomers thereof, all solvates and hydrates thereof, all crystalline and non-crystalline forms thereof, and all salts thereof, particularly pharmaceutically-acceptable salts. This subset of compounds of formula I, inclusive of the proviso set forth above, are designated the compounds of subset AP of formula I, or the compounds of formula I-AP. Metabolites and prodrugs of the compounds of formula I-AP are also embraced by the invention. In one embodiment, R₁ₐₚ is unsubstituted phenyl. In another embodiment, R₁ₐₚ is substituted phenyl. In another embodiment, R₁ₐₚ is substituted phenyl bearing one substituent. In another embodiment, R₁ₐₚ is substituted phenyl bearing two substituents. In another embodiment, X is O. In another embodiment, X is NR₆. In another embodiment, R₃ is H or C₁-C₄ alkyl. In another embodiment, R₆ is H or C₁-C₄ alkyl.

In another embodiment, the present invention relates to methods of using the compounds of formula I-AP to inhibit SSAO enzyme activity (whether the enzyme activity is due either to soluble SSAO enzyme or membrane-bound VAP-1 protein, or due to both) and/or inhibit binding to VAP-1 protein. The compounds can be used for a method of inhibiting SSAO activity or inhibiting binding to VAP-1 in vitro, by supplying the compound to the in vitro environment in an amount sufficient to inhibit SSAO activity or inhibit binding to VAP-1. The compounds can also be used for a method of inhibiting SSAO activity or inhibiting binding to VAP-1 in vivo, that is, in a living organism, such as a vertebrate, mammal, or human, by administering the compounds to the organism in an amount sufficient to inhibit SSAO activity or inhibit binding to VAP-1. In another embodiment, the present invention relates to methods of using the compounds of formula I-AP to treat inflammation or immune disorders. In another embodiment, the present invention relates to methods of using the compounds of formula I-AP to suppress or reduce inflammation, or to suppress or reduce an inflammatory response. In another embodiment, the present invention relates to methods of treating inflammation, by administering one or more of the compounds described in formula I-AP in a therapeutically effective amount, or in an amount sufficient to treat inflammation. In another embodiment, the present invention relates to methods of treating immune or autoimmune disorders, by administering one or more of the compounds described in formula I-AP in a therapeutically effective amount, or in an amount sufficient to treat the immune or autoimmune disorder.

In one embodiment, the present invention relates to compounds of formula I-B: 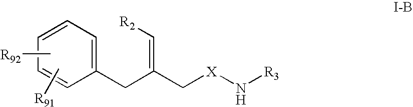
wherein:
R₂ is independently chosen from H, C₁-C₄ alkyl, Cl, F, or CF₃;
R₉₁ and R₉₂ are independently chosen from H, F, Br, Cl, I, C₁-C₄ alkyl, and C₁-C₄ alkoxy;
X is independently chosen from O or NR₆;
R₃ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₆ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
   including all stereoisomers thereof, all E/Z (cis/trans) isomers thereof, all solvates and hydrates thereof, all crystalline and non-crystalline forms thereof, and all salts thereof, particularly pharmaceutically-acceptable salts. This subset of compounds of formula I are designated the compounds of subset B of formula I, or the compounds of formula I-B. Metabolites and prodrugs of the compounds of formula I-B are also embraced by the invention. In one embodiment, X is O. In a preferred embodiment, X is NR₆. In another preferred embodiment, R₃ is H or C₁-C₄ alkyl. In another preferred embodiment, R₆ is H or C₁-C₄ alkyl. In another preferred embodiment, R₉₁ is H. In another preferred embodiment, R₉₂ is H. In another preferred embodiment, R₉₁ and R₉₂ are both H.

In another embodiment, the present invention relates to methods of using the compounds of formula I-B to inhibit SSAO enzyme activity (whether the enzyme activity is due either to soluble SSAO enzyme or membrane-bound VAP-1 protein, or due to both) and/or inhibit binding to VAP-1 protein. The compounds can be used for a method of inhibiting SSAO activity or inhibiting binding to VAP-1 in vitro, by supplying the compound to the in vitro environment in an amount sufficient to inhibit SSAO activity or inhibit binding to VAP-1. The compounds can also be used for a method of inhibiting SSAO activity or inhibiting binding to VAP-1 in vivo, that is, in a living organism, such as a vertebrate, mammal, or human, by administering the compounds to the organism in an amount sufficient to inhibit SSAO activity or inhibit binding to VAP-1. In another embodiment, the present invention relates to methods of using the compounds of formula I-B to treat inflammation or immune disorders. In another embodiment, the present invention relates to methods of using the compounds of formula I-B to suppress or reduce inflammation, or to suppress or reduce an inflammatory response. In another embodiment, the present invention relates to methods of treating inflammation, by administering one or more of the compounds described in formula I-B in a therapeutically effective amount, or in an amount sufficient to treat inflammation. In another embodiment, the present invention relates to methods of treating immune or autoimmune disorders, by administering one or more of the compounds described in formula I-B in a therapeutically effective amount, or in an amount sufficient to treat the immune or autoimmune disorder.

In one embodiment, the present invention relates to compounds of formula I-C: 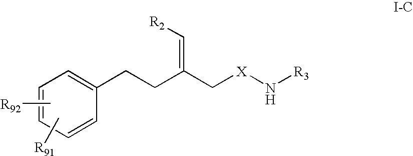
wherein:
R₂ is independently chosen from H, C₁-C₄ alkyl, Cl, F, or CF₃;
R₉₁ and R₉₂ are independently chosen from H, F, Br, Cl, I, C₁-C₄ alkyl, and C₁-C₄ alkoxy;
X is independently chosen from O or NR₆;
R₃ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₆ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
   including all stereoisomers thereof, all E/Z (cis/trans) isomers thereof, all solvates and hydrates thereof, all crystalline and non-crystalline forms thereof, and all salts thereof, particularly pharmaceutically-acceptable salts. This subset of compounds of formula I are designated the compounds of subset C of formula I, or the compounds of formula I-C. Metabolites and prodrugs of the compounds of formula I-C are also embraced by the invention. In one embodiment, X is O. In another preferred embodiment, X is NR₆. In another preferred embodiment, R₃ is H or C₁-C₄ alkyl. In another preferred embodiment, R₆ is H or C₁-C₄ alkyl. In another preferred embodiment, R₉₁ is H. In another preferred embodiment, R₉₂ is H. In another preferred embodiment, R₉₁ and R₉₂ are both H.

In another embodiment, the present invention relates to methods of using the compounds of formula I-C to inhibit SSAO enzyme activity (whether the enzyme activity is due either to soluble SSAO enzyme or membrane-bound VAP-1 protein, or due to both) and/or inhibit binding to VAP-1 protein. The compounds can be used for a method of inhibiting SSAO activity or inhibiting binding to VAP-1 in vitro, by supplying the compound to the in vitro environment in an amount sufficient to inhibit SSAO activity or inhibit binding to VAP-1. The compounds can also be used for a method of inhibiting SSAO activity or inhibiting binding to VAP-1 in vivo, that is, in a living organism, such as a vertebrate, mammal, or human, by administering the compounds to the organism in an amount sufficient to inhibit SSAO activity or inhibit binding to VAP-1. In another embodiment, the present invention relates to methods of using the compounds of formula I-C to treat inflammation or immune disorders. In another embodiment, the present invention relates to methods of using the compounds of formula I-C to suppress or reduce inflammation, or to suppress or reduce an inflammatory response. In another embodiment, the present invention relates to methods of treating inflammation, by administering one or more of the compounds described in formula I-C in a therapeutically effective amount, or in an amount sufficient to treat inflammation. In another embodiment, the present invention relates to methods of treating immune or autoimmune disorders, by administering one or more of the compounds described in formula I-C in a therapeutically effective amount, or in an amount sufficient to treat the immune or autoimmune disorder.

In another embodiment, the present invention relates to methods of using the compounds of formula I, I-P, I-A, I-AP, I-B, and/or I-C to inhibit SSAO enzyme activity (whether the enzyme activity is due either to soluble SSAO enzyme or membrane-bound VAP-1 protein, or due to both) and/or inhibit binding to VAP-1 protein. The compounds can be used for a method of inhibiting SSAO activity or inhibiting binding to VAP-1 in vitro, by supplying the compound to the in vitro environment in an amount sufficient to inhibit SSAO activity or inhibit binding to VAP-1. The compounds can also be used for a method of inhibiting SSAO activity or inhibiting binding to VAP-1 in vivo, that is, in a living organism, such as a vertebrate, mammal, or human, by administering the compounds to the organism in an amount sufficient to inhibit SSAO activity or inhibit binding to VAP-1. In another embodiment, the present invention relates to methods of using the compounds of formula I, I-P, I-A, I-AP, I-B, and/or I-C to treat inflammation or immune disorders. In another embodiment, the present invention relates to methods of using the compounds of formula I, I-P, I-A, I-AP, I-B, and/or I-C to suppress or reduce inflammation, or to suppress or reduce an inflammatory response. In another embodiment, the present invention relates to methods of treating inflammation, by administering one or more of the compounds described in formula I, I-P, I-A, I-AP, I-B, and/or I-C in a therapeutically effective amount, or in an amount sufficient to treat inflammation. In another embodiment, the present invention relates to methods of treating immune or autoimmune disorders, by administering one or more of the compounds described in formula I, I-P, I-A, I-AP, I-B, and/or I-C in a therapeutically effective amount, or in an amount sufficient to treat the immune or autoimmune disorder.

In another embodiment, the present invention relates to compounds of general formula II: 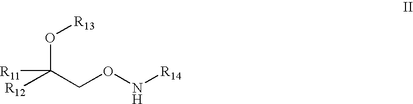
R₁₁ and R₁₂ are independently chosen from the group consisting of H, C₁-C₄ alkyl,
C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₁₃ and R₁₄ are independently chosen from H, C₁-C₄ alkyl, C₃-C₈ cycloalkyl, C₆-C₁₀ aryl,
C₆-C₁₄ aralkyl, C₄-C₉ heteroaryl, C₆-C₁₄ substituted aryl and C₅-C₁₄ substituted heteroaryl;
including all stereoisomers thereof, all E/Z (cis/trans) isomers thereof, all solvates and hydrates thereof, all crystalline and non-crystalline forms thereof, and all salts thereof, particularly pharmaceutically-acceptable salts. Metabolites and prodrugs of the compounds of formula II are also embraced by the invention. In one embodiment, R₁₁ is H. In another embodiment, R₁₂ is unsubstituted phenyl. In another embodiment, R₁₂ is substituted phenyl. In another embodiment, R₁₃ is H or C₁-C₄ alkyl. In another embodiment, R₁₄ is H or C₁-C₄ alkyl.

In another embodiment, the present invention relates to methods of using the compounds of formula II to inhibit SSAO enzyme activity (whether the enzyme activity is due either to soluble SSAO enzyme or membrane-bound VAP 1 protein, or due to both) and/or inhibit binding to VAP-1 protein. The compounds can be used for a method of inhibiting SSAO activity or inhibiting binding to VAP-1 in vitro, by supplying the compound to the in vitro environment in an amount sufficient to inhibit SSAO activity or inhibit binding to VAP-1. The compounds can also be used for a method of inhibiting SSAO activity or inhibiting binding to VAP-1 in vivo, that is, in a living organism, such as a vertebrate, mammal, or human, by administering the compounds to the organism in an amount sufficient to inhibit SSAO activity or inhibit binding to VAP-1. In another embodiment, the present invention relates to methods of using the compounds of formula II to treat inflammation or immune disorders. In another embodiment, the present invention relates to methods of using the compounds of formula II to suppress or reduce inflammation, or to suppress or reduce an inflammatory response. In another embodiment, the present invention relates to methods of treating inflammation, by administering one or more of the compounds described in formula II in a therapeutically effective amount, or in an amount sufficient to treat inflammation. In another embodiment, the present invention relates to methods of treating immune or autoimmune disorders, by administering one or more of the compounds described in formula II in a therapeutically effective amount, or in an amount sufficient to treat the immune or autoimmune disorder.

In another embodiment, the present invention relates to compounds of general formula III: 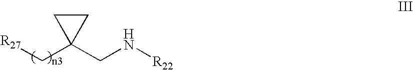
wherein R₂₇ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl, C₅-C₁₄ substituted heteroaryl, R₂₃—(CH₂)ₙ—, and R₂₄—Y₂—(CH₂)—;
R₂₂ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
n is independently 1 or 2;
n3 is independently 0, 1, or 2;
Y₂ is independently S or O; and
R₂₃ and R₂₄ are independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
   including all stereoisomers thereof, all E/Z (cis/trans) isomers thereof, all solvates and hydrates thereof, all crystalline and non-crystalline forms thereof, and all salts thereof, particularly pharmaceutically-acceptable salts. Metabolites and prodrugs of the compounds of formula III are also embraced by the invention. In one embodiment, R₂₇ is unsubstituted phenyl. In another embodiment, R₂₇ is substituted phenyl. In another embodiment, R₂₂ is H or C₁-C₄ alkyl. In another embodiment, R₂₂ is not H. In another embodiment, R₂₂ is C₁-C₄ alkyl. In another embodiment, R₂₂ is methyl or ethyl.

In another embodiment, the present invention relates to methods of using the compounds of formula III to inhibit SSAO enzyme activity (whether the enzyme activity is due either to soluble SSAO enzyme or membrane-bound VAP 1 protein, or due to both) and/or inhibit binding to VAP-1 protein. The compounds can be used for a method of inhibiting SSAO activity or inhibiting binding to VAP-1 in vitro, by supplying the compound to the in vitro environment in an amount sufficient to inhibit SSAO activity or inhibit binding to VAP-1. The compounds can also be used for a method of inhibiting SSAO activity or inhibiting binding to VAP-1 in vivo, that is, in a living organism, such as a vertebrate, mammal, or human, by administering the compounds to the organism in an amount sufficient to inhibit SSAO activity or inhibit binding to VAP-1. In another embodiment, the present invention relates to methods of using the compounds of formula III to treat inflammation or immune disorders. In another embodiment, the present invention relates to methods of using the compounds of formula III to suppress or reduce inflammation, or to suppress or reduce an inflammatory response. In another embodiment, the present invention relates to methods of treating inflammation, by administering one or more of the compounds described in formula III in a therapeutically effective amount, or in an amount sufficient to treat inflammation. In another embodiment, the present invention relates to methods of treating immune or autoimmune disorders, by administering one or more of the compounds described in formula III in a therapeutically effective amount, or in an amount sufficient to treat the immune or autoimmune disorder.

In another embodiment, the present invention relates to compounds of general formula III-A 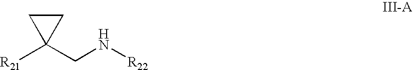
wherein R₂₁ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl, C₅-C₁₄ substituted heteroaryl, R₂₃—(CH₂)ₙ—, and R₂₄—Y₂—(CH₂)—;
R₂₂ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
n is independently 1 or 2;
Y₂ is independently S or O; and
R₂₃ and R₂₄ are independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl; including all stereoisomers thereof, all E/Z (cis/trans) isomers thereof, all solvates and hydrates thereof, all crystalline and non-crystalline forms thereof, and all salts thereof, particularly pharmaceutically-acceptable salts. Metabolites and prodrugs of the compounds of formula III-A are also embraced by the invention. In one embodiment, R₂, is unsubstituted phenyl. In another embodiment, R₂, is substituted phenyl. In another embodiment, R₂₂ is H or C₁-C₄ alkyl. In another embodiment, R₂₂ is not H. In another embodiment, R₂₂ is C₁-C₄ alkyl. In another embodiment, R₂₂ is methyl or ethyl.

In another embodiment, the present invention relates to methods of using the compounds of formula III-A to inhibit SSAO enzyme activity (whether the enzyme activity is due either to soluble SSAO enzyme or membrane-bound VAP 1 protein, or due to both) and/or inhibit binding to VAP-1 protein. The compounds can be used for a method of inhibiting SSAO activity or inhibiting binding to VAP-1 in vitro, by supplying the compound to the in vitro environment in an amount sufficient to inhibit SSAO activity or inhibit binding to VAP-1. The compounds can also be used for a method of inhibiting SSAO activity or inhibiting binding to VAP-1 in vivo, that is, in a living organism, such as a vertebrate, mammal, or human, by administering the compounds to the organism in an amount sufficient to inhibit SSAO activity or inhibit binding to VAP-1. In another embodiment, the present invention relates to methods of using the compounds of formula III-A to treat inflammation or immune disorders. In another embodiment, the present invention relates to methods of using the compounds of formula III-A to suppress or reduce inflammation, or to suppress or reduce an inflammatory response. In another embodiment, the present invention relates to methods of treating inflammation, by administering one or more of the compounds described in formula III-A in a therapeutically effective amount, or in an amount sufficient to treat inflammation. In another embodiment, the present invention relates to methods of treating immune or autoimmune disorders, by administering one or more of the compounds described in formula III-A in a therapeutically effective amount, or in an amount sufficient to treat the immune or autoimmune disorder.

In another embodiment, the present invention relates to compounds of general formula III-B: 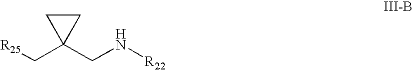
wherein R₂₅ is independently chosen from C₆-C₁₀ aryl, C₆-C₁₄ aralkyl, C₄-C₉ heteroaryl, C₆-C₁₄ substituted aryl, and C₅-C₁₄ substituted heteroaryl; and R₂₂ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl; including all stereoisomers thereof, all E/Z (cis/trans) isomers thereof, all solvates and hydrates thereof, all crystalline and non-crystalline forms thereof, and all salts thereof, particularly pharmaceutically-acceptable salts. Metabolites and prodrugs of the compounds of formula III-B are also embraced by the invention. In one embodiment, R₂₅ is unsubstituted phenyl. In another embodiment, R₂₅ is substituted phenyl. In another embodiment, R₂₂ is H or C₁-C₄ alkyl. In another embodiment, R₂₂ is not H. In another embodiment, R₂₂ is C₁-C₄ alkyl. In another embodiment, R₂₂ is methyl or ethyl.

In another embodiment, the present invention relates to methods of using the compounds of formula III-B to inhibit SSAO enzyme activity (whether the enzyme activity is due either to soluble SSAO enzyme or membrane-bound VAP 1 protein, or due to both) and/or inhibit binding to VAP-1 protein. The compounds can be used for a method of inhibiting SSAO activity or inhibiting binding to VAP-1 in vitro, by supplying—the compound to the in vitro environment in an amount sufficient to inhibit SSAO activity or inhibit binding to VAP-1. The compounds can also be used for a method of inhibiting SSAO activity or inhibiting binding to VAP-1 in vivo, that is, in a living organism, such as a vertebrate, mammal, or human, by administering the compounds to the organism in an amount sufficient to inhibit SSAO activity or inhibit binding to VAP-1. In another embodiment, the present invention relates to methods of using the compounds of formula III-B to treat inflammation or immune disorders. In another embodiment, the present invention relates to methods of using the compounds of formula III-B to suppress or reduce inflammation, or to suppress or reduce an inflammatory response. In another embodiment, the present invention relates to methods of treating inflammation, by administering one or more of the compounds described in formula III-B in a therapeutically effective amount, or in an amount sufficient to treat inflammation. In another embodiment, the present invention relates to methods of treating immune or autoimmune disorders, by administering one or more of the compounds described in formula III-B in a therapeutically effective amount, or in an amount sufficient to treat the immune or autoimmune disorder.

In another embodiment, the present invention relates to compounds of general formula III-C: 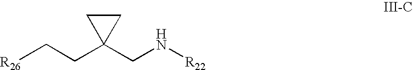
wherein R₂₆ is independently chosen from C₆-C₁₀ aryl, C₆-C₁₄ aralkyl, C₄-C₉ heteroaryl, C₆-C₁₄ substituted aryl, and C₅-C₁₄ substituted heteroaryl; and
R₂₂ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
   including all stereoisomers thereof, all E/Z (cis/trans) isomers thereof, all solvates and hydrates thereof, all crystalline and non-crystalline forms thereof, and all salts thereof, particularly pharmaceutically-acceptable salts. Metabolites and prodrugs of the compounds of formula III-C are also embraced by the invention. In one embodiment, R₂₆ is unsubstituted phenyl. In another embodiment, R₂₆ is substituted phenyl. In another embodiment, R₂₂ is H or C₁-C₄ alkyl. In another embodiment, R₂₂ is not H. In another embodiment, R₂₂ is C₁-C₄ alkyl. In another embodiment, R₂₂ is methyl or ethyl.

In another embodiment, the present invention relates to methods of using the compounds of formula III-C to inhibit SSAO enzyme activity (whether the enzyme activity is due either to soluble SSAO enzyme or membrane-bound VAP 1 protein, or due to both) and/or inhibit binding to VAP-1 protein. The compounds can be used for a method of inhibiting SSAO activity or inhibiting binding to VAP-1 in vitro, by supplying the compound to the in vitro environment in an amount sufficient to inhibit SSAO activity or inhibit binding to VAP-1. The compounds can also be used for a method of inhibiting SSAO activity or inhibiting binding to VAP-1 in vivo, that is, in a living organism, such as a vertebrate, mammal, or human, by administering the compounds to the organism in an amount sufficient to inhibit SSAO activity or inhibit binding to VAP-1. In another embodiment, the present invention relates to methods of using the compounds of formula III-C to treat inflammation or immune disorders. In another embodiment, the present invention relates to methods of using the compounds of formula III-C to suppress or reduce inflammation, or to suppress or reduce an inflammatory response. In another embodiment, the present invention relates to methods of treating inflammation, by administering one or more of the compounds described in formula III-C in a therapeutically effective amount, or in an amount sufficient to treat inflammation. In another embodiment, the present invention relates to methods of treating immune or autoimmune disorders, by administering one or more of the compounds described in formula III-C in a therapeutically effective amount, or in an amount sufficient to treat the immune or autoimmune disorder.

In another embodiment, the inflammatory disease or immune disorder to be treated by one or more of the compounds of formulas I, I-P, I-A, I-AP, I-B, I-C, II, III, III-A, III-B, and/or III-C of the present invention is selected from the group consisting of multiple sclerosis (including chronic multiple sclerosis); synovitis; systemic inflammatory sepsis; inflammatory bowel diseases; Crohn's disease; ulcerative colitis; Alzheimer's disease; vascular dementia; atherosclerosis; rheumatoid arthritis; juvenile rheumatoid arthritis; pulmonary inflammatory conditions; asthma; skin inflammatory conditions and diseases; contact dermatitis; liver inflammatory and autoimmune conditions; autoimmune hepatitis; primary biliary cirrhosis; sclerosing cholangitis; autoimmune cholangitis; alcoholic liver disease; Type I diabetes and/or complications thereof; Type II diabetes and/or complications thereof; atherosclerosis; chronic heart failure; congestive heart failure; ischemic diseases such as stroke and/or complications thereof; and myocardial infarction and/or complications thereof. In another embodiment, the inflammatory disease or immune disorder to be treated by the present invention is multiple sclerosis (including chronic multiple sclerosis). In another embodiment, the inflammatory disease or immune disorder to be treated by the present invention is the inflammatory complications resulting from stroke.

A compound of formula I, I-P, I-A, I-AP, I-B, I-C, II, III, III-A, III-B, or III-C as described above can be administered singly in a therapeutically effective amount. A compound of formula I, I-P, I-A, I-AP, I-B, I-C, II, III, III-A, III-B, or III-C as described above can be administered with one or more additional compounds of formulas I, I-P, I-A, I-AP, I-B, I-C, II, III, III-A, III-B, or III-C, in a therapeutically effective amount. When administered in combination, the compounds can be administered in amounts that would therapeutically effective were the compounds to be administered singly. Alternatively, when administered in combination, any or all of compounds can be administered in amounts that would not be therapeutically effective were the compounds to be administered singly, but which are therapeutically effective in combination. One or more compounds of formulas I, I-P, I-A, I-AP, I-B, I-C, II, III, III-A, III-B, or 111-C can also be administered with other compounds not included in formulas I, I-P, I-A, I-AP, I-B, I-C, II, III, III-A, III-B, or III-C; the compounds can be administered in amounts that are therapeutically effective when used as single drugs, or in amounts which are not therapeutically effective as single drugs, but which are therapeutically effective in combination. Also provided are pharmaceutically acceptable compositions comprising a therapeutically effective amount of one or more of the compounds disclosed herein or a therapeutically effective combination of two or more of the compounds disclosed herein, including the compounds of formulas I, I-P, I-A, I-AP, I-B, I-C, II, III, III-A, III-B, and/or III-C above, and a pharmaceutically acceptable carrier; and human unit dosages thereof.

A compound of formula I, I-P, I-A, I-AP, I-B, I-C, II, III, III-A, III-B, and/or III-C as described above can be prepared as an isolated pharmaceutical composition, and administered as an isolated pharmaceutical composition in conjunction with vehicles or other isolated compounds. That is, a compound of formula I, I-P, I-A, I-AP, I-B, I-C, II, III, III-A, III-B, and/or III-C as described above can be isolated from other compounds (e.g., a compound which is discovered in a library screening assay can be purified out of the library, or synthesized de novo as a single compound). The degree of purification can be 90%, 95%, 99%, or whatever percentage of purity is required for pharmaceutical use of the compound. The isolated compound can then be combined with pharmaceutically acceptable vehicles, or can be combined with one or more isolated compounds of formulas I, I-P, I-A, I-AP, I-B, I-C, II, III, III-A, III-B, and/or III-C, or with another therapeutic substance. A compound of formula I, I-P, I-A, I-AP, I-B, I-C, II, III, III-A, III-B, and/or III-C as described above can be administered orally, in a pharmaceutical human unit dosage formulation.

In another embodiment, the invention embraces compounds of formula I for use in therapy. In another embodiment, the invention embraces compounds of formula I for manufacture of a medicament for treatment of inflammatory diseases. In another embodiment, the invention embraces compounds of formula I for manufacture of a medicament for treatment of immune or autoimmune diseases. In another embodiment, the invention embraces compounds of formula I for manufacture of a medicament for treatment of multiple sclerosis or chronic multiple sclerosis. In another embodiment, the invention embraces compounds of formula I for manufacture of a medicament for treatment of ischemic diseases (such as stroke) or the sequelae of ischemic diseases.

In another embodiment, the invention embraces compounds of formula I-P for use in therapy. In another embodiment, the invention embraces compounds of formula I-P for manufacture of a medicament for treatment of inflammatory diseases. In another embodiment, the invention embraces compounds of formula I-P for manufacture of a medicament for treatment of immune or autoimmune diseases. In another embodiment, the invention embraces compounds of formula I-P for manufacture of a medicament for treatment of multiple sclerosis or chronic multiple sclerosis. In another embodiment, the invention embraces compounds of formula I-P for manufacture of a medicament for treatment of ischemic diseases (such as stroke) or the sequelae of ischemic diseases.

In another embodiment, the invention embraces compounds of formula I-A for use in therapy. In another embodiment, the invention embraces compounds of formula I-A for manufacture of a medicament for treatment of inflammatory diseases. In another embodiment, the invention embraces compounds of formula I-A for manufacture of a medicament for treatment of immune or autoimmune diseases. In another embodiment, the invention embraces compounds of formula I-A for manufacture of a medicament for treatment of multiple sclerosis or chronic multiple sclerosis. In another embodiment, the invention embraces compounds of formula I-A for manufacture of a medicament for treatment of ischemic diseases (such as stroke) or the sequelae of ischemic diseases.

In another embodiment, the invention embraces compounds of formula I-AP for use in therapy. In another embodiment, the invention embraces compounds of formula I-AP for manufacture of a medicament for treatment of inflammatory diseases. In another embodiment, the invention embraces compounds of formula I-AP for manufacture of a medicament for treatment of immune or autoimmune diseases. In another embodiment, the invention embraces compounds of formula I-AP for manufacture of a medicament for treatment of multiple sclerosis or chronic multiple sclerosis. In another embodiment, the invention embraces compounds of formula I-AP for manufacture of a medicament for treatment of ischemic diseases (such as stroke) or the sequelae of ischemic diseases.

In another embodiment, the invention embraces compounds of formula I-B for use in therapy. In another embodiment, the invention embraces compounds of formula I-B for manufacture of a medicament for treatment of inflammatory diseases. In another embodiment, the invention embraces compounds of formula I-B for manufacture of a medicament for treatment of immune or autoimmune diseases. In another embodiment, the invention embraces compounds of formula I-B for manufacture of a medicament for treatment of multiple sclerosis or chronic multiple sclerosis. In another embodiment, the invention embraces compounds of formula I-B for manufacture of a medicament for treatment of ischemic diseases (such as stroke) or the sequelae of ischemic diseases.

In another embodiment, the invention embraces compounds of formula I-C for use in therapy. In another embodiment, the invention embraces compounds of formula I-C for manufacture of a medicament for treatment of inflammatory diseases. In another embodiment, the invention embraces compounds of formula I-C for manufacture of a medicament for treatment of immune or autoimmune diseases. In another embodiment, the invention embraces compounds of formula I-C for manufacture of a medicament for treatment of multiple sclerosis or chronic multiple sclerosis. In another embodiment, the invention embraces compounds of formula I-C for manufacture of a medicament for treatment of ischemic diseases (such as stroke) or the sequelae of ischemic diseases.

In another embodiment, the invention embraces compounds of formula II for use in therapy. In another embodiment, the invention embraces compounds of formula II for manufacture of a medicament for treatment of inflammatory diseases. In another embodiment, the invention embraces compounds of formula II for manufacture of a medicament for treatment of immune or autoimmune diseases. In another embodiment, the invention embraces compounds of formula II for manufacture of a medicament for treatment of multiple sclerosis or chronic multiple sclerosis. In another embodiment, the invention embraces compounds of formula II for manufacture of a medicament for treatment of ischemic diseases (such as stroke) or the sequelae of ischemic diseases.

In another embodiment, the invention embraces compounds of formula III for use in therapy. In another embodiment, the invention embraces compounds of formula III for manufacture of a medicament for treatment of inflammatory diseases. In another embodiment, the invention embraces compounds of formula III for manufacture of a medicament for treatment of immune or autoimmune diseases. In another embodiment, the invention embraces compounds of formula III for manufacture of a medicament for treatment of multiple sclerosis or chronic multiple sclerosis. In another embodiment, the invention embraces compounds of formula III for manufacture of a medicament for treatment of ischemic diseases (such as stroke) or the sequelae of ischemic diseases.

In another embodiment, the invention embraces compounds of formula III-A for use in therapy. In another embodiment, the invention embraces compounds of formula III-A for manufacture of a medicament for treatment of inflammatory diseases. In another embodiment, the invention embraces compounds of formula III-A for manufacture of a medicament for treatment of immune or autoimmune diseases. In another embodiment, the invention embraces compounds of formula III-A for manufacture of a medicament for treatment of multiple sclerosis or chronic multiple sclerosis. In another embodiment, the invention embraces compounds of formula III-A for manufacture of a medicament for treatment of ischemic diseases (such as stroke) or the sequelae of ischemic diseases.

In another embodiment, the invention embraces compounds of formula III-B for use in therapy. In another embodiment, the invention embraces compounds of formula III-B for manufacture of a medicament for treatment of inflammatory diseases. In another embodiment, the invention embraces compounds of formula III-B for manufacture of a medicament for treatment of immune or autoimmune diseases. In another embodiment, the invention embraces compounds of formula III-B for manufacture of a medicament for treatment of multiple sclerosis or chronic multiple sclerosis. In another embodiment, the invention embraces compounds of formula III-B for manufacture of a medicament for treatment of ischemic diseases (such as stroke) or the sequelae of ischemic diseases.

In another embodiment, the invention embraces compounds of formula III-C for use in therapy. In another embodiment, the invention embraces compounds of formula III-C for manufacture of a medicament for treatment of inflammatory diseases. In another embodiment, the invention embraces compounds of formula III-C for manufacture of a medicament for treatment of immune or autoimmune diseases. In another embodiment, the invention embraces compounds of formula III-C for manufacture of a medicament for treatment of multiple sclerosis or chronic multiple sclerosis. In another embodiment, the invention embraces compounds of formula III-C for manufacture of a medicament for treatment of ischemic diseases (such as stroke) or the sequelae of ischemic diseases.

### BRIEF DESCRIPTION OF THE DRAWINGS

FIG. 1A depicts the effect of the compound of Example 2 ((2-phenylallyl)hydrazine) on monoclonal antibody-induced arthritis disease development as assessed by arthritis score, versus phosphate buffer saline (PBS) control and methotrexate.

FIG. 1B depicts the effect of the compound of Example 2 ((2-phenylallyl)hydrazine) on monoclonal antibody-induced arthritis disease development as assessed by paw measurements, versus PBS control and methotrexate.

FIG. 1C depicts the effect of the compound of Example 2 ((2-phenylallyl)hydrazine) on monoclonal antibody-induced arthritis disease development as assessed by percent incidence, versus PBS control and methotrexate.

FIG. 2A depicts the effect of the allylamine (AA) compound of Example 18 (mofegiline) on experimental autoimmune encephalitis (EAE) development as assessed by clinical severity, versus vehicle control and methotrexate.

FIG. 2B depicts the effect of the allylamine (AA) compound of Example 18 (mofegiline) on EAE development as assessed by percent incidence, versus vehicle control and methotrexate.

FIG. 2C depicts the effect of the allylamine (AA) compound of Example 18 (mofegiline) on EAE development as assessed by body weight, versus vehicle control and methotrexate.

FIG. 3A depicts the effect of the compound of Example 2 ((2-phenylallyl)hydrazine) on EAE development as assessed by percent incidence, versus vehicle control.

FIG. 3B depicts the effect of the compound of Example 2 ((2-phenylallyl)hydrazine) on EAE development as assessed by clinical severity, versus vehicle control.

FIG. 4A depicts the effect of the compounds of Examples 2 and 8 versus PBS on induced paw inflammation. The various triangle, square, and diamond symbols in the figure represent individual test animals.

FIG. 4B depicts the effect of the compounds of Examples 2 and 8 versus phosphate-buffered saline on induced paw inflammation. The symbols in the figure represent individual mice.

FIGS. 5A and 5B depict oral availability studies in mice and rats. FIG. 5A depicts oral availability studies in mice; FIG. 5B depicts oral availability studies in rats. Compounds were administered to mice and rats by oral gavage at a concentration of 50 mg/kg in phosphate buffered saline (PBS). Plasma was collected at the times indicated in the figures, and the concentration of inhibitor was determined using the SSAO colorimetric assay described in Example 14.

FIG. 6 depicts in vivo inhibition of SSAO activity. Dose response effect of a single oral dose administration of the compound of example 8 on SSAO activity in rat aorta and lung 4 hrs after treatment (mean S.E.M.). ED₅₀ Values: Aorta—5 mg/kg; Lung—0.72 mg/kg; n=5.

FIG. 7 depicts the effect of blockage of SSAO/VAP-1 on binding between peripheral blood mononuclear cells (PBMCs) and high endothelial cells (HEC). FIG. 7A is a control experiment showing the effect of various compounds used for treatment on the adhesion of PBMCs to mock-transfected high endothelial cells; a non-treated control is included for comparison (MNT). FIG. 7B is a control experiment showing the effect of various compounds used for treatment on the adhesion of PBMCs to high endothelial cells transfected with VAP-1; a non-treated control is included for comparison (VNT). MNT: Mock transfected cells, not treated; VNT: VAP-1-transfected cells, not treated; VAP-1: cells treated with anti-VAP1 antibody; Ex2: cells treated with compound of example 2; Ex8: cells treated with compound of example 8; Semic: cells treated with semicarbazide; Clog: cells treated with clorgyline; Parg: cells treated with pargyline. IC₁₀₀ values: Semicarbazide (SSAO)—500 μM; Clorgyline (MAO-A)—250 μM; Pargyline (MAO-B)—200 μM; Compound of example 2—150 nM; Compound of example 8-250 nM; n=6.

FIG. 8 shows RT-PCR amplification of 18S rRNA and TNFα from mouse paw samples. FIG. 8A: RT-PCR amplification of cDNA from paws and digits of representative animals. FIG. 8B.: The right hind paws from all animals from the three different groups were removed and the total RNA was isolated and used in qualitative RT-PCR studies as described in Example 17. The densitometry units (DU) from the TNF and 18S bands were determined for each sample and their ratios were averaged (±SD). “Compound 2” indicates the compound of example 2.

FIG. 9 shows the ameliorating effect of administration of (2-phenylallyl)hydrazine in a model of chronic multiple sclerosis. FIG. 9A depicts the mean clinical score of mice treated with PBS (phosphate buffered saline) versus mice treated with (2-phenylallyl)hydrazine. FIG. 9B depicts the percentage of disease incidence in mice treated with PBS (phosphate buffered saline) versus mice treated with (2-phenylallyl)hydrazine. FIG. 9C depicts the percentage of mice with chronic disease in mice treated with PBS (phosphate buffered saline) versus mice treated with (2-phenylallyl)hydrazine. FIG. 9D depicts the total number of relapses in mice treated with PBS (phosphate buffered saline) versus mice treated with (2-phenylallyl)hydrazine.

FIG. 10 shows the effect of SSAO inhibition on reduction of paw edema after therapeutic administration. Animals received the compound of example 2 ((2-phenyallyl)hydrazine, 30 mg/kg, p.o.), indomethacin (3 mg/kg, p.o.) or PBS one hour after carrageenan injection (arrow). Paw volumes were recorded at the indicated times and expressed as percent of the volume before injection. N=8 animals/group; *p<0.05.

FIG. 11 shows data indicating that SSAO inhibitor reduces paw volume (FIG. 11A) and PGE2 levels (FIG. 11B) in paw exudates. Eight animals per group received (via oral administration) PBS, indomethacin (3 mg/kg) or the compound of example 2 ((2-phenyallyl)hydrazine, 50 mg/kg) one hour prior to injection of 0.5% carrageenan in the right hind footpad. Dexamethasone (3 mg/kg) was administered i.p. as well one hour prior to carrageenan injection. Three hours after carrageenan injection, animals were sacrificed, their paw exudates were collected and PGE2 levels were determined by ELISA. Asterisks indicate the following p values: *p<0.05; **p<0.01.

FIG. 12 shows data indicating that SSAO/VAP-1 inhibition prolongs survival, reduces disease symptoms and improves histological scores in a murine colitis model. Oxazolone-induced colitis is a mouse model that resembles human ulcerative colitis. Mice were presensitized with 3% oxazolone (day 0), and five days later were intrarectally challenged with 1% oxazolone (day 5). Treatment with the compound of example 2 ((2-phenylallyl)hydrazine, 10 mg/kg, twice a day, i.p.) or PBS (twice a day, i.p.) was initiated on day 0. Animals in the EtOH group were presensitized with 3% oxazolone, followed by an intrarectal administration of 50% EtOH (vehicle) on day 5. FIG. 12A shows the effect on survival, while FIG. 12B shows the effect on body weight. In FIG. 12A, n=10, p<0.05; squares indicate EtOH group, triangles indicate PBS group, circles indicate the group receiving the compound of example 2. In FIG. 12B, n=10, asterisk * indicates p value of p<0.05; squares indicate EtOH group, regular triangles indicate PBS group, inverted triangles indicate group receiving the compound of example 2.

FIG. 13 shows histological assessments of colitis in mice with oxazolone-induced colitis two days after intrarectal administration of 1% oxazolone (day 7). Colons were fixed and stained with H/E. Treatment with the compound of example 2 ((2-phenylallyl)hydrazine, 20 mg/kg/day), or PBS was initiated on day 0. N=10 animals/treatment group. Data is for scores from section 2. Unpaired t tests were calculated using GraphPad Prism software (San Diego, Calif.). Double asterisks ** indicate p<0.01.

FIG. 14 shows that an SSAO inhibitor prolongs survival after therapeutic administration. Mice were presensitized with 3% oxazolone (day 0), and five days later were intrarectally challenged with 1% oxazolone (day 5). Treatment with the compound of example 2 ((2-phenyallyl)hydrazine, 10 mg/kg, twice a day, i.p.) or PBS (twice a day, i.p.) was initiated on day 6. N=10, p<0.05; squares indicate data points for animals receiving PBS; diamonds indicate data points for animals receiving the compound of example 2.

FIG. 15 shows that oral dosing with SSAO inhibitor reduces LPS-induced cytokine production and lethality. Eight female mice per group received i.p. injections of 5 mg/kg LPS. Vehicle (PBS) and the compound of example 2 ((2-phenylallyl)hydrazine, 50 mg/kg) were dosed orally one hour prior to LPS administration. Dexamethasone (3 mg/kg) was administered i.p. at the same time. Blood was collected 1, 2, 4, and 8 hours after LPS injection and circulating TNF-α and IL-6 levels were measured by ELISA (R&D Systems). Asterisk * indicates p<0.01. PBS data is shown in the clear boxes, data for the compound of example 2 is shown in the gray-shaded boxes, data for dexamethasone is shown in the black-filled boxes.

FIG. 16 shows the results of an experiment where female mice received LPS (2 mg/kg) administered i.p. together with 300 mg/kg D-galactosamine. The compound of example 2 ((2-phenylallyl)hydrazine) was delivered by oral gavage of 30 mg/kg at the time of the challenge (1×), or dosed two times at 0 and 8 hours post LPS injection (2×). Survival data for the first 14 hours is presented. The survival was 40% for mice treated with PBS and 60 and 80% for mice treated once or twice with the compound of example 2, respectively.

### MODES FOR CARRYING OUT THE INVENTION

The present invention relates to various compounds which are useful for inhibiting SSAO enzyme activity (where the enzyme activity is due either to soluble SSAO enzyme or membrane-bound VAP-1 protein, or due to both) and/or inhibition of binding to membrane-bound VAP-1 protein. The present invention also relates to methods of using various compounds to inhibit SSAO enzyme activity (where the enzyme activity is due either to soluble SSAO enzyme or membrane-bound VAP-1 protein, or due to both) and/or inhibit binding to VAP-1 protein. The present invention also relates to methods of using various compounds to treat inflammation or immune disorders, and to reduce or suppress inflammation and/or inflammatory responses.

Compounds for use in the invention can be assayed for SSAO inhibitory activity by the protocol in Example 14 below. The substrate specificity of SSAO versus monoamine oxidase partially overlap. Thus it is preferable to use compounds which are specifically inhibit SSAO over monoamine oxidase. The specificity of the compounds for SSAO inhibitory activity versus MAO-A and MAO-B inhibitory activity can be assayed by the protocol in Example 15 below. Compounds for use in the invention have an inhibitory activity (IC o) against SSAO of about<1 μM, more preferably of about 100 nM, and more preferably of about 10 nM. Preferably, compounds for use in the invention also have a specificity for SSAO versus MAO-A of about 10, more preferably about 100, more preferably about 500 (where specificity for SSAO versus MAO-A is defined as the ratio of the IC₅₀ of a compound for MAO-A to the IC₅₀ of the same compound for SSAO; that is, a compound with an IC₅₀ of 10 μM for MAO-A and an IC₅₀ of 20 nM for SSAO has a specificity of 500 for SSAO versus MAO-A). Compounds for use in the invention also have a specificity for SSAO versus MAO-B of about 10, more preferably of about 100, more preferably of about 500 (where specificity for SSAO versus MAO-B is defined as the ratio of the IC₅₀ of a compound for MAO-B to the IC₅₀ of the same compound for SSAO). Table 1 below provides experimental values for several of the compounds for use in the invention.

The term “inhibit binding to VAP-1 protein” is meant to indicate inhibition (which can include partial to complete inhibition) of binding between, for example, a cell expressing the SSAO/VAP-1 protein on its surface, and a binding partner of SSAO/VAP-1 protein. Such binding occurs, for example, when a cell expressing the SSAO/VAP-1 protein on its surface interacts with another cell expressing a binding partner of SSAO/VAP-1 protein, such as a high endothelial cell (HEC). Thus “inhibit binding to VAP-1 protein” embraces inhibition of adhesion between a cell expressing the SSAO/VAP-1 protein on its surface, and another cell expressing a binding partner of SSAO/VAP-1 protein. Such adhesion events include, for example, cell rolling. As this disclosure (including the examples) clearly indicates, such inhibition can occur either in vitro or in vivo.

The invention includes all salts of the compounds described herein, as well as methods of using such salts of the compounds. The invention also includes all pure (non-salt) compounds of any salt of a compound named herein, as well as other salts of any salt of a compound named herein. In one embodiment, the salts of the compounds comprise pharmaceutically acceptable salts. Pharmaceutically acceptable salts are those salts which retain the biological activity of the free compounds and which are not biologically or otherwise undesirable. The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, can also be prepared.

The invention also includes all stereoisomers of the compounds, including diastereomers and enantiomers, as well as mixtures of stereoisomers, including, but not limited to, racemic mixtures. Unless stereochemistry is explicitly indicated in a chemical structure or chemical name, the chemical structure or chemical name is intended to embrace all possible stereoisomers of the compound depicted. Also, while the general formulas I, I-P, I-A, I-AP, I-B, and I-C are drawn with only one of the cis-trans isomers depicted (with R₁ and R₂ depicted as cis to each other), the drawing is intended to embrace both the compounds with R₁ and R₂ in the cis position as well as R₁ and R₂ in the trans position (that is, the single drawing is used to represent both the E and Z isomers, although only one isomer is drawn).

The term “alkyl” refers to saturated aliphatic groups including straight-chain, branched-chain, cyclic groups, and combinations thereof, having the number of carbon atoms specified, or if no number is specified, having up to 12 carbon atoms. “Straight-chain alkyl” or “linear alkyl” groups refers to alkyl groups that are neither cyclic nor branched, commonly designated as “n-alkyl” groups. Examples of alkyl groups include, but are not limited to, groups such as methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl. Cycloalkyl groups can consist of one ring, including, but not limited to, groups such as cycloheptyl, or multiple fused rings, including, but not limited to, groups such as adamantyl or norbornyl.

“Substituted alkyl” refers to alkyl groups substituted with one or more substituents including, but not limited to, groups such as halogen (fluoro, chloro, bromo, and iodo), alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of substituted alkyl groups include, but are not limited to, —CF₃, —CF₂—CF₃, and other perfluoro and perhalo groups; —CH₂—OH; —CH₂CH₂CH(NH₂)CH₃, etc.

The term “alkenyl” refers to unsaturated aliphatic groups including straight-chain (linear), branched-chain, cyclic groups, and combinations thereof, having the number of carbon atoms specified, or if no number is specified, having up to 12 carbon atoms, which contain at least one double bond (—C═C—). Examples of alkenyl groups include, but are not limited to, —CH₂—CH═CH—CH₃; and —CH₂—CH₂-cyclohexenyl, where the ethyl group can be attached to the cyclohexenyl moiety at any available carbon valence. The term “alkynyl” refers to unsaturated aliphatic groups including straight-chain (linear), branched-chain, cyclic groups, and combinations thereof, having the number of carbon atoms specified, or if no number is specified, having up to 12 carbon atoms, which contain at least one triple bond (—C═C—). “Hydrocarbon chain” or “hydrocarbyl” refers to any combination of straight-chain, branched-chain, or cyclic alkyl, alkenyl, or alkynyl groups, and any combination thereof. “Substituted alkenyl,” “substituted alkynyl,” and “substituted hydrocarbon chain” or “substituted hydrocarbyl” refer to the respective group substituted with one or more substituents, including, but not limited to, groups such as halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

“Aryl” or “Ar” refers to an aromatic carbocyclic group having a single ring (including, but not limited to, groups such as phenyl) or two or more condensed rings (including, but not limited to, groups such as naphthyl or anthryl), and includes both unsubstituted and substituted aryl groups. Aryls, unless otherwise specified, contain from 6 to 12 carbon atoms in the ring portion. A preferred range for aryls is from 6 to 10 carbon atoms in the ring portion. “Substituted aryls” refers to aryls substituted with one or more substituents, including, but not limited to, groups such as alkyl, alkenyl, alkynyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. “Aralkyl” designates an alkyl-substituted aryl group, where any aryl can attached to the alkyl; the alkyl portion is a straight or branched chain of 1 to 6 carbon atoms, preferably the alkyl chain contains 1 to 3 carbon atoms. When an aralkyl group is indicated as a substituent, the aralkyl group can be connected to the remainder of the molecule at any available valence on either its alkyl moiety or aryl moiety; e.g., the tolyl aralkyl group can be connected to the remainder of the molecule by replacing any of the five hydrogens on the aromatic ring moiety with the remainder of the molecule, or by replacing one of the alpha-hydrogens on the methyl moiety with the remainder of the molecule. Preferably, the aralkyl group is connected to the remainder of the molecule via the alkyl moiety.

A preferred aryl group is phenyl, which can be substituted or unsubstituted. Preferred substitutents for substituted phenyl groups are lower alkyl (—C₁-C₄ alkyl), or a halogen (chlorine (—Cl), bromine (—Br), iodine (—I), or fluorine (—F); preferred halogen substituents for pheny groups are chlorine and fluorine), hydroxy (—OH), or lower alkoxy (—C₁-C₄ alkoxy), such as methoxy, ethoxy, propyloxy (propoxy) (either n-propoxy or i-propoxy), and butoxy (either n-butoxy, i-butoxy, sec-butoxy, or tert-butoxy); a preferred alkoxy substituent is methoxy. Substituted phenyl groups preferably have one or two substituents; more preferably, one substituent.

“Heteroalkyl,” “heteroalkenyl,” and “heteroalkynyl” refer to alkyl, alkenyl, and alkynyl groups, respectively, that contain the number of carbon atoms specified (or if no number is specified, having up to 12 carbon atoms) which contain one or more heteroatoms as part of the main, branched, or cyclic chains in the group. Heteroatoms include, but are not limited to, N, S, O, and P; N and O are preferred. Heteroalkyl, heteroalkenyl, and heteroalkynyl groups may be attached to the remainder of the molecule either at a heteroatom (if a valence is available) or at a carbon atom. Examples of heteroalkyl groups include, but are not limited to, groups such as —O—CH₃, —CH₂—O—CH₃, —CH₂—CH₂—O—CH₃, —S—CH₂—CH₂—CH₃, —CH₂—CH(CH₃)—S—CH₃, —CH₂—CH₂—NH—CH₂—CH₂—, 1-ethyl-6-propylpiperidino, and morpholino. Examples of heteroalkenyl groups include, but are not limited to, groups such as —CH═CH—NH—CH(CH₃)—CH₂—. “Heteroaryl” or “HetAr” refers to an aromatic carbocyclic group having a single ring (including, but not limited to, examples such as pyridyl, imidazolyl, thiophene, or furyl) or two or more condensed rings (including, but not limited to, examples such as indolizinyl or benzothienyl) and having at least one hetero atom, including, but not limited to, heteroatoms such as N, O, P, or S, within the ring. Unless otherwise specified, heteroalkyl, heteroalkenyl, heteroalkynyl, and heteroaryl groups have between one and five heteroatoms and between one and twelve carbon atoms. “Substituted heteroalkyl,” “substituted heteroalkenyl,” “substituted heteroalkynyl,” and “substituted heteroaryl” groups refer to heteroalkyl, heteroalkenyl, heteroalkynyl, and heteroaryl groups substituted with one or more substituents, including, but not limited to, groups such as alkyl, alkenyl, alkynyl, benzyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of such substituted heteroalkyl groups include, but are not limited to, piperazine, substituted at a nitrogen or carbon by a phenyl or benzyl group, and attached to the remainder of the molecule by any available valence on a carbon or nitrogen, —NH—SO₂-phenyl, —NH—(C═O)O-alkyl, —NH—(C═O)O-alkyl-aryl, and —NH—(C═O)-alkyl. If chemically possible, the heteroatom(s) and/or the carbon atoms of the group can be substituted. The heteroatom(s) can also be in oxidized form, if chemically possible.

The term “alkoxy” as used herein refers to an alkyl, alkenyl, alkynyl, or hydrocarbon chain linked to an oxygen atom and having the number of carbon atoms specified, or if no number is specified, having up to 12 carbon atoms. Examples of alkoxy groups include, but are not limited to, groups such as methoxy, ethoxy, propyloxy (propoxy) (either n-propoxy or i-propoxy), and butoxy (either n-butoxy, i-butoxy, sec-butoxy, or tert-butoxy). The groups listed in the preceding sentence are preferred alkoxy groups; a particularly preferred alkoxy substituent is methoxy.

The terms “halo” and “halogen” as used herein refer to the Group VIIa elements (Group 17 elements in the 1990 IUPAC Periodic Table, IUPAC Nomenclature of Inorganic Chemistry, Recommendations 1990) and include Cl, Br, F and I substituents. Preferred halogen substituents are Cl and F.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 3rd Ed. (John Wiley & Sons, Inc., New York). Amino protecting groups include, but are not limited to, mesitylenesulfonyl (Mts), benzyloxycarbonyl (CBz or Z), t-butyloxycarbonyl (Boc), t-butyldimethylsilyl (TBS or TBDMS), 9-fluorenylmethyloxycarbonyl (Fmoc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, dimethyl dimethoxybenzil, 5-bromo-7-nitroindolinyl, and the like. Hydroxylprotecting groups include, but are not limited to, Fmoc, TBS, photolabile protecting groups (such as nitroveratryl oxymethyl ether (Nvom)), Mom (methoxy methyl ether), and Mem (methoxy ethoxy methyl ether), NPEOC (4-nitrophenethyloxycarbonyl) and NPEOM (4-nitrophenethyloxymethyloxycarbonyl).

General Synthetic Methods

The compounds of the formulas described herein can be prepared by various methods. Compounds of formula I are conveniently prepared using a 1,2-substituted propene as starting material (see Examples 1-10 below). The methyl group of the propene can be derivatized with a good leaving group (LG), e.g., by bromination to yield a 3-bromo-1,2-substituted propene.

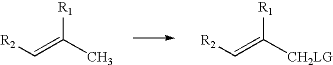

Reaction with an N-protected hydroxylamine compound, HON(R₃)(PG) where PG is a protecting group (e.g., N-Boc hydroxylamine (t-butyl N-hydroxy carbamate)), or with a mono-protected hydrazine compound H(R₆)N—N(R₃)(PG) (e.g., N-Boc hydrazine (t-butyl carbazate)), yields compounds of formula I with X=O or NR₆, respectively.

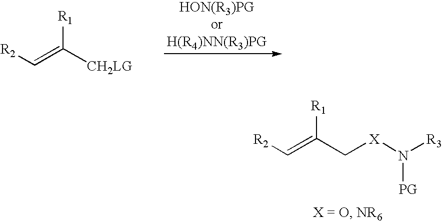

Certain compounds of fomula I (e.g., compounds of of formula I-B or formula I-C) are conveniently prepared by the following synthetic route, starting from commercially available benzeneacetic acid (phenylacetic acid, alpha-tolylic acid; Aldrich; corresponding to n=0 in the following reaction scheme) or 3-phenylpropionic acid (hydrocinnamic acid; Aldrich; corresponding to n=1 in the following reaction scheme). Other compounds of formula I-B and formula I-C can be synthesized using phenyl-substituted phenylacetic acid or phenyl-substituted 3-phenylpropionic acid.

The acid is converted into (2-benzyl)acrylic acid methyl ester or (2-phenethyl)acrylic acid methyl ester according to the procedures indicated in Hin, B. et al., J. Org. Chem. 67:7365-7368 (2002) (see procedure for preparation of 8b from 6b, benzeneacetic acid at pages 7367-7368). Briefly, dicyclohexylcarbodiimide (DCC) in methylene chloride is added to a solution of benzeneacetic acid or 3-phenylpropionic acid, Meldrum's acid (2,2-dimethyl-1,3-dioxane-4,6-dione), and dimethylaminopyridine and reacted at 0° C. overnight. The solution is filtered, washed, and dried, acidified, and then NaBH₄ is added and the reaction allowed to proceed overnight at 0° C. The solution is washed, dried, and concentrated, and the product purified by recrystallization or silica gel chromatography, to yield the 5-substituted Meldrum's acid intermediate. The Meldrum's acid intermediate is then stirred with dimethyl methyleneimmonium iodide in anhydrous methanol at 65° C. overnight. The reaction mixture is concentrated, taken up in diethyl ether, washed, dried and concentrated to give the (2-benzyl)acrylic acid methyl ester or (2-phenethyl)acrylic acid methyl ester.

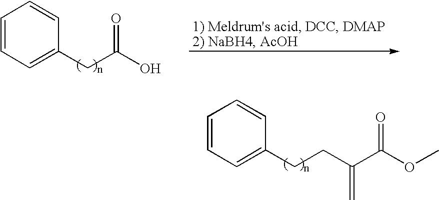

The products depicted above, (2-benzyl)acrylic acid methyl ester (n=0) or (2-phenethyl)acrylic acid methyl ester (n=1) compounds are then subjected to a DIBAL reduction, resulting in reduction of the esters to alcohols (2-phenethyl-2-propene-1-ol (n=0) or 2-(3-phenylpropyl)-2-propene-1-ol (n=1)):

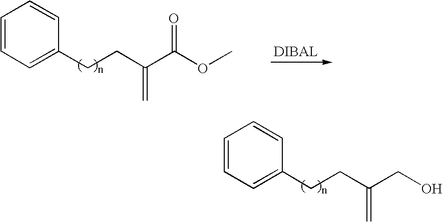

The alcohols are then subjected to a Mitsunobu reaction using N-(Boc-amino)phthalimide (N′-Boc-N,N-phthaloylhydrazine; commercially available from Fluka, Switzerland) (see Brosse et al., Tetrahedron Lett. 41, 205 (2000); Brosse et al., J. Org. Chem. 66, 2869 (2001); Brosse et al., Journal of Organic Chemistry, 65(14), 4370-4374 (2000); see also Example 10 below; see also Hughes, D. L. Org. Reac. 42:335-656 (1992) and Mitsunobu, O.et al., J. Am. Chem. Soc. 94:679 (1972)), to yield the following product: 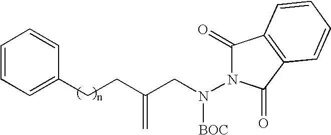
followed by removal of the protecting groups to yield:

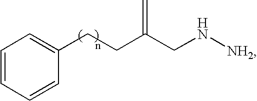

### (2-phenethylallyl)hydrazine (n=0) and [2-(3-phenylpropyl)allyl]hydrazine (n=1)

Compounds of formula II are conveniently prepared by several methods. One such method utilizes a 1,1-disubstituted ethylene oxide as starting material, which is reacted with a compound of the formula HON(R₁₄)PG, where PG is a protecting group and R₁₄ is as indicated in formula II (see Examples 11 and 12 below). 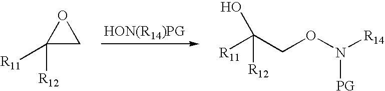
The hydroxyloxygen can then be subjected to further derivatization, and the protecting group removed at the end of the synthesis.

Compounds of formula III are conveniently prepared by various methods. One such method (see Example 13 below) utilizes a benzyl cyanide (phenylacetonitrile) starting material. The phenyl ring may be optionally substituted. Other groups, such as alkyl, cycloalkyl, aryl (substituted or unsubstituted), or heteroaryl (substituted or unsubstituted) can be used, e.g., 2-pyridylacetonitrile. The benzyl cyanide is treated with a strong, sterically hindered base, such as potassium bis(trimethylsilyl)amide, followed by addition of an ethyl compound substituted at the 1 and 2 positions with good leaving groups, such as 1,2-dibromoethane. This results in a 1-phenyl, 1-cyano cyclopropane compound. 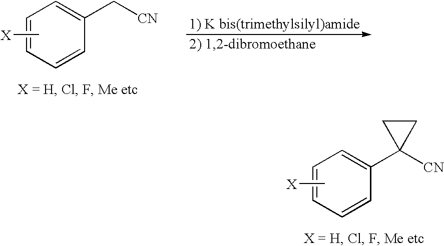
The cyano group can then be reduced by methods known in the art (such as by addition of lithium aluminum hydride) to produce the corresponding amine compound.

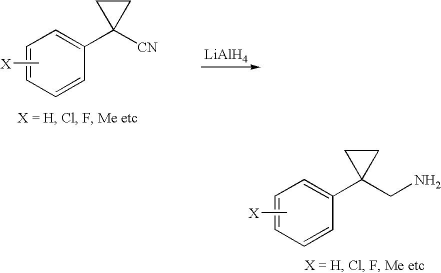

The amino group thus produced can be reacted with a wide variety of reagents, e.g. alkyl bromides, aldehydes or ketones followed by reduction, acyl compounds followed by reduction, etc., to introduce the R₂₂ group onto the nitrogen.

Another route for synthesis of compounds of formula III, which is useful for preparation of compounds of formula III-B and formula III-C, inter alia, is depicted in the following scheme (where Ar designates an aryl group, such as a C₆-C₁₀ substituted or unsubstituted aryl group). Cyclopropanecarbonitrile (cyclopropyl cyanide; Aldrich) is reacted with base (such as lithium diisopropylamide, LDA), followed by reaction with a substituted alkyl bromide; the nitrile group is then reduced to an amino group.

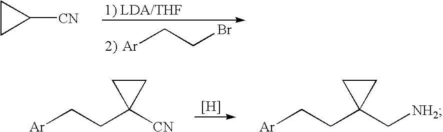

For example, reaction of (2-bromoethyl)benzene with base, followed by cyclopropanecarbonitrile, yields the intermediate (1-phenethyl)cyclopropanecarbonitrile, which is reduced to (1-phenethylcyclopropyl)methylamine; reaction of benzyl bromide (α-bromotoluene) with base, followed by cyclopropanecarbonitrile, yields the intermediate 1-benzylcyclopropanecarbonitrile, which is reduced to (1-benzylcyclopropyl)methylamine.

Methods of Use

The compounds discussed herein can be used in a variety of manners. One such use is in treatment of inflammation, inflammatory diseases, inflammatory responses, and certain other diseases, as described in more detail below under “Treatment of Diseases.” Other uses include inhibiting SSAO enzyme activity and/or VAP-1 binding activity or VAP-1 amine oxidase activity, both in vivo and in vitro. An example of in vitro use of the compounds is use in assays, such as conventional assays or high-throughput screening assays.

Treatment of Diseases

Compounds discussed herein are useful for treating inflammation and inflammatory conditions, and for treating immune and autoimmune disorders. The compounds are also useful for treating one or more of a variety of diseases caused by or characterized by inflammation or immune disorders. Thus the compounds can be used to treat diseases caused by inflammation, and can also be used to treat diseases which cause inflammation. The compounds are used to treat mammals, preferably humans. “Treating” a disease with the compounds discussed herein is defined as administering one or more of the compounds discussed herein, with or without additional therapeutic agents, in order to prevent, reduce, or eliminate either the disease or one or more symptoms of the disease, or to retard the progression of the disease or of one or more symptoms of the disease, or to reduce the severity of the disease or of one or more symptoms of the disease. “Therapeutic use” of the compounds discussed herein is defined as using one or more of the compounds discussed herein to treat a disease, as defined above. A “therapeutically effective amount” of a compound is an amount of the compound, which, when administered to a subject, is sufficient to prevent, reduce, or eliminate either the disease or one or more symptoms of the disease, or to retard the progression of the disease or of one or more symptoms of the disease, or to reduce the severity of the disease or of one or more symptoms of the disease. A “therapeutically effective amount” can be given in one or more administrations.

The subjects which can be treated with the compounds and methods of the invention include vertebrates, preferably mammals, more preferably humans.

Diseases which can be treated with the compound and methods of the invention include inflammation, inflammatory responses, inflammatory diseases and immune disorders. It should be noted that inflammatory diseases can be caused by immune disorders, and that immune disorders are often accompanied by inflammation, and therefore both inflammation and immune disorders may be treated simultaneously by the compounds and methods of the invention. Diseases which can be treated with the compounds and methods of the invention include, but are not limited to, multiple sclerosis (including chronic multiple sclerosis); synovitis; systemic inflammatory sepsis; inflammatory bowel diseases; Crohn's disease; ulcerative colitis; Alzheimer's disease; atherosclerosis; rheumatoid arthritis; juvenile rheumatoid arthritis; pulmonary inflammatory conditions; asthma; skin inflammatory conditions and diseases; contact dermatitis; liver inflammatory and autoimmune conditions; autoimmune hepatitis; primary biliary cirrhosis; sclerosing cholangitis; autoimmune cholangitis; alcoholic liver disease; Type I diabetes and/or complications thereof; Type II diabetes and/or complications thereof; atherosclerosis; ischemic diseases such as stroke and/or complications thereof; and myocardial infarction. In another embodiment, the inflammatory disease or immune disorder to be treated by the present invention is multiple sclerosis. In another embodiment, the inflammatory disease or immune disorder to be treated by the present invention is chronic multiple sclerosis. In another embodiment, the inflammatory disease or immune disorder to be treated by the present invention is the inflammatory complications resulting from stroke.

Modes of Administration

The compounds described for use in the present invention can be administered to a mammalian, preferably human, subject via any route known in the art, including, but not limited to, those disclosed herein. Methods of administration include but are not limited to, intravenous, oral, intraarterial, intramuscular, topical, via inhalation (e.g. as mists or sprays), via nasal mucosa, subcutaneous, transdermal, intraperitoneal, gastrointestinal, and directly to a specific or affected organ. Oral administration is a preferred route of administration. The compounds described for use herein can be administered in the form of tablets, pills, powder mixtures, capsules, granules, injectables, creams, solutions, suppositories, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

The compounds of the present invention may be administered in an effective amount within the dosage range of about 0.1 μg/kg to about 300 mg/kg, or within about 1.0 μg/kg to about 40 mg/kg body weight, or within about 1.0 μg/kg to about 20 mg/kg body weight, preferably between about 1.0 μg/kg to about 10 mg/kg body weight. Compounds of the present invention may be administered in a single daily dose, or the total daily dosage may be administered in divided dosage of two, three or four times daily.

The pharmaceutical dosage form which contains the compounds described herein is conveniently admixed with a non-toxic pharmaceutical organic carrier or a non-toxic pharmaceutical inorganic carrier. Typical pharmaceutically-acceptable carriers include, for example, mannitol, urea, dextrans, lactose, potato and maize starches, magnesium stearate, talc, vegetable oils, polyalkylene glycols, ethyl cellulose, poly(vinylpyrrolidone), calcium carbonate, ethyl oleate, isopropyl myristate, benzyl benzoate, sodium carbonate, gelatin, potassium carbonate, silicic acid, and other conventionally employed acceptable carriers. The pharmaceutical dosage form can also contain non-toxic auxiliary substances such as emulsifying, preserving, or wetting agents, and the like. A suitable carrier is one which does not cause an intolerable side effect, but which allows the compound(s) to retain its pharmacological activity in the body. Formulations for parenteral and nonparenteral drug delivery are known in the art and are set forth in Remington: The Science and Practice of Pharmacy, 20th Edition, Lippincott, Williams & Wilkins (2000). Solid forms, such as tablets, capsules and powders, can be fabricated using conventional tableting and capsule-filling machinery, which is well known in the art. Solid dosage forms, including tablets and capsules for oral administration in unit dose presentation form, can contain any number of additional non-active ingredients known to the art, including such conventional additives as excipients; desiccants; colorants; binding agents, for example syrup, acacia, gelatin, sorbitol, tragacanth, or polyvinylpyrollidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulfate. The tablets can be coated according to methods well known in standard pharmaceutical practice. Liquid forms for ingestion can be formulated using known liquid carriers, including aqueous and non-aqueous carriers such as sterile water, sterile saline, suspensions, oil-in-water and/or water-in-oil emulsions, and the like. Liquid formulations can also contain any number of additional non-active ingredients, including colorants, fragrance, flavorings, viscosity modifiers, preservatives, stabilizers, and the like. For parenteral administration, the compounds for use in the invention can be administered as injectable dosages of a solution or suspension of the compound in a physiologically acceptable diluent or sterile liquid carrier such as water, saline, or oil, with or without additional surfactants or adjuvants. An illustrative list of carrier oils would include animal and vegetable oils (e.g., peanut oil, soy bean oil), petroleum-derived oils (e.g., mineral oil), and synthetic oils. In general, for injectable unit doses, sterile liquids such as water, saline, aqueous dextrose and related sugar solutions, and ethanol and glycol solutions such as propylene glycol or polyethylene glycol are preferred liquid carriers.

The pharmaceutical unit dosage chosen is preferably fabricated and administered to provide a defined final concentration of drug in the blood, tissues, organs, or other targeted region of the body. The optimal effective concentration of the compounds of the invention can be determined empirically and will depend on the type and severity of the disease, route of administration, disease progression and health, mass and body area of the patient. Such determinations are within the skill of one in the art.

The compounds for use in the invention can be administered as the sole active ingredient, or can be administered in combination with another active ingredient.

Kits

The invention also provides articles of manufacture and kits containing materials useful for treating diseases such as inflammatory diseases, autoimmune diseases, multiple sclerosis (including chronic multiple sclerosis); synovitis; systemic inflammatory sepsis; inflammatory bowel diseases; Crohn's disease; ulcerative colitis; Alzheimer's disease; atherosclerosis; rheumatoid arthritis; juvenile rheumatoid arthritis; pulmonary inflammatory conditions; asthma; skin inflammatory conditions and diseases; contact dermatitis; liver inflammatory and autoimmune conditions; autoimmune hepatitis; primary biliary cirrhosis; sclerosing cholangitis; autoimmune cholangitis; alcoholic liver disease; Type I diabetes and/or complications thereof; Type II diabetes and/or complications thereof; atherosclerosis; ischemic diseases such as stroke and/or complications thereof; and myocardial infarction; or for inhibiting SSAO enzyme activity (whether the enzyme activity is due either to soluble SSAO enzyme or membrane-bound VAP-1 protein, or due to both) and/or inhibiting binding to VAP-1 protein. The article of manufacture comprises a container with a label. Suitable containers include, for example, bottles, vials, and test tubes. The containers may be formed from a variety of materials such as glass or plastic. The container holds a composition having an active agent which is effective for treating diseases or for inhibiting SSAO or VAP-1 enzyme activity or binding to VAP-1 protein. The active agent in the composition is one or more of the compounds of formulas I, I-P, I-A, I-AP, I-B, I-C, II, III, III-A, III-B, and/or III-C. The label on the container indicates that the composition is used for treating diseases such as inflammatory or autoimmune diseases, or for inhibiting SSAO or VAP-1 enzyme activity or binding to VAP-1 protein, and may also indicate directions for either in vivo or in vitro use, such as those described above.

The invention also provides kits comprising any one or more of the compounds of formulas I, I-P, I-A, I-AP, I-B, I-C, II, III, III-A, III-B, and/or III-C. In some embodiments, the kit of the invention comprises the container described above. In other embodiments, the kit of the invention comprises the container described above and a second container comprising a buffer. It may further include other materials desirable from a commercial and user standpoint, including other buffers, diluents, filters, needles, syringes, and package inserts with instructions for performing any methods described herein (such as methods for treating autoimmune or inflammatory diseases, and methods for inhibiting SSAO or VAP-1 enzyme activity or binding to VAP-1 protein).

In other aspects, the kits may be used for any of the methods described herein, including, for example, to treat an individual with autoimmune or inflammatory disease, such as multiple sclerosis or ischemic disease (such as stroke) and the sequelae thereof.

The disclosures of all publications, patents, patent applications and published patent applications referred to herein by an identifying citation are hereby incorporated herein by reference in their entirety.

The invention will be further understood by the following nonlimiting examples. The phrase “the compound of example X” whereused herein refers to the compound in the title of the example; e.g., the compound of example 8 refers to N-[2-(4′-fluorophenyl)-allyl]-hydrazine hydrochloride, while the compound of example 10 refers to (E)-1-fluoro-2-phenyl-3-hydrazinopropene hydrochloride. It should be noted that, while the compounds are typically described as salts, the disclosure expressly includes the non-salt forms of the compounds, as well as any other salt of the compound; e.g., N-[2-(4′-fluorophenyl)-allyl]-hydrazine hydrochloride is intended as a disclosure of the non-salt compound N-[2-(4′-fluorophenyl)-allyl]-hydrazine.

### EXAMPLES

### Example 1

### O-(2-phenyl-allyl)-hydroxylamine Hydrochloride

A mixture of α-methylstyrene (17.73 g, 150 mmol) and N-bromosuccinimide (17.80 g, 100 mmol) was dissolved in CCl₄ (20 ml) in a flask fitted with a reflux condenser and magnetic stirrer. It was heated until the mixture was refluxing. The reaction was moderated to maintain a gentle reflux for a period of 3 hrs, and then cooled to room temperature. The precipitated succinimide was separated by filtration. The filtrate was concentrated in vacuo. The residue was purified via column chromatography (silica gel, 100% hexanes). The product, α-bromomethylstyrene, was obtained as an oil (13.0 g, 66%).

A solution of HONHBoc (5.99 g, 45.0 mmol) and NaOH (1.8 g, 45.0 mmol) in MeOH (20 ml was stirred at room temperature for 1 hr. To this mixture was added dropwise a solution of α-bromomethylstyrene (5.91 g, 30.0 mmol) in MeOH (5 ml). The resulting reaction mixture was kept gentle reflux under N₂ for overnight. The mixture was concentrated in vacuo. The residue was diluted with H₂O, and then extracted with EtOAc (3×20 ml). The combined organic layers were dried (MgSO₄), and filtered. The filtrate was concentrated in vacuo. The residue was purified via column chromatography (silica gel, 10% EtOAc/hexanes). N-tert-butyloxycarbonyl-O-(2-phenylallyl)hydroxylamine (4.0 g) was obtained as a white solid. ¹H NMR (CDCl₃, 300 MHz) δ 7.25-7.58 (m, 5H), 7.65 (s, 1H), 5.42 (s, 1H), 4.79 (s, 2H), 1.52 (s, 9H).

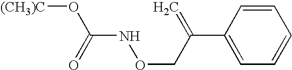

To a solution of N-tert-butyloxycarbonyl-O-(2-phenylallyl) hydroxylamine (2.0 g, 8.0 mmol) in ether (5 ml) was added 1M HCl in ether (20 ml, 20 mmol). The resulting mixture was stirred under N₂ for 3 hrs at room temperature. The precipitate was collected by filtration, washed with ether (3×20 ml), and then dried in vacuo. O-(2-Phenyl-allyl)-hydroxylamine hydrochloride was obtained (0.80 g, 67%). mp 101-102° C. ¹H NMR (D₂O, 300 MHz) δ 7.25-7.52 (m, 5H), 5.75 (s, 1H), 5.45 (s, 1H), 4.89 (s, 2H).

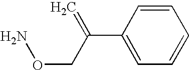

### Example 2

### 2-(Phenyl-allyl)-hydrazine Hydrochloride

“The compound of example 2” refers to (2-phenyl-2-propenyl)hydrazine (CAS Registry No. 65814-30-4), also named (2-phenylallyl)hydrazine, which is the product of this example (see the last structure of this example). A mixture of NH₂NHBoc (3.96 g, 30 mmol) and Et₃N (3.04 g, 30 mmol) in MeOH (15 ml) was stirred for 20 min. at room temperature. To this mixture was added α-bromomethylstyrene (2.96 g, 15 mmol). The resulting mixture was gently refluxed and monitored by TLC. After refluxing for about 3 hrs, the TLC indicated that the reaction was completed. The reaction mixture was concentrated in vacuo. The residue was purified via column chromatography (silica gel, 10% EtOAc/hexanes) to provide 3-(N′-tert-butyloxycarbonylhydrazino)-2-phenyl propene (1.34 g, 39%) as a white solid. ¹H NMR (CDCl₃, 300 MHz) δ 7.26-7.55 (m, 5H), 5.50 (s, 1H), 5.30 (s, 1H), 3.90 (s, 2H), 1.52 (s, 9H).

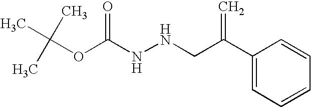

To a solution of 3-(N′-tert-butyloxycarbonylhydrazino)-2-phenyl propene (1.0 g, 4 mmol) in ether (5 ml) was added a solution of 1M HCl in ether (20 ml, 20 mmol). The solution was stirred under N₂ at room temperature for 5 hrs. TLC showed that the reaction was not completed. Thus, the mixture was concentrated in vacuo. The residue was dissolved in anhydrous MeOH (3 ml). To this solution was added a solution of 1M HCl in ether (20 ml, 20 mmol). The resulting mixture was stirred under N₂ at room temperature for 3 hrs. TLC showed that the reaction was completed. The solid formed was collected by filtration, washed with ether, and then dried in vacuo. A white crystalline solid was obtained (0.36 g, 48%). mp: 153-154.5° C. ¹H NMR (D₂O, 300 MHz) δ 7.25-7.42 (m, 5), 5.60 (s, 1H), 5.40 (s, 1H), 4.05 (s, 2H). This compound is designated as the compound of example 2, (2-phenyl-2-propenyl)hydrazine (also named (2-phenylallyl)hydrazine), depicted immediately below.

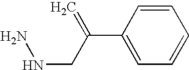

### Example 3

### N-[2-(4′-chlorophenyl)-allyl]-hydrazine Hydrochloride

A mixture of tert-butyl carbazate (6.61 g, 50 mmole) and Et₃N (5.06 g, 50 mmole) in MeOH (40 ml) was stirred at room temperature for 20 min. To this stirred mixture was added 4-chloro-α-bromomethylstyrene (prepared according to the procedures described in Tetrahedron Lett. Yamanaka, M. et al. 2002, 43, 2403-2406) (6.95 g, 30 mmol). The resulting mixture was heated to reflux and monitored by TLC. TLC showed that the reaction was completed after refluxing for 3 hrs. The mixture was concentrated in vacuo. The residue was purified via column chromatography (silica gel, 5% EtOAc/Hexanes). 3-(N′-tert-butyloxycarbonylhydrazino)-2-(4′-chlorophenyl) propene was obtained as a white solid (2.70 g, 20%). ¹H NMR (CDCl₃, 300 MHz) δ 7.42 (d, J=8.4 Hz, 2H), 7.29 (d, J=8.4 Hz, 2H), 5.45. (s, 1H), 5.28 (s, 1H), 3.85 (s, 2H), 1.45 (s, 9H).

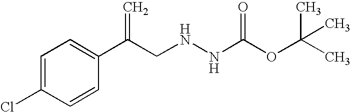

To a solution of 3-(N′-tert-butyloxycarbonylhydrazino)-2-(4′-chlorophenyl) propene (0.78 g, 2.76 mmol) in MeOH (4 ml) was added a solution of HCl in ether (2 M. 5.0 ml, 10 mmol). The solution was stirred at room temperature for overnight. The reaction mixture was concentrated in vacuo. The resulting solid was washed with ether, which afforded 2-(4′-chlorophenyl)-allyl hydrazine hydrochloride as a white solid (0.61 g, 100%). Mp: 124-126° C. ¹HNMR (D₂O, 300 MHz) δ 7.40 (d, J=9 Hz, 2H), 7.35 (d, J=9 Hz, 2H), 5.65 (s, 1H), 5.43 (s, 1H), 4.06 (s, 2H).

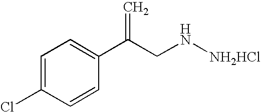

### Example 4

### N-[2-(4′-chlor-phenyl)-allyl]-N-methyl-hydrazine Hydrochloride

A mixture of 3-(N′-tert-butyloxycarbonylhydrazino)-2-(4′-chlorophenyl) propene (see Example 3) (1.00 g, 3.54 mmol) and N,N-diisopropylethylamine (0.91 g, 7.08 mmol) in DMF (10 ml) was stirred under N₂ at room temperature for 20 min before MeI (1.00 g, 7.08 mmol) was added dropwise. The resulting mixture was stirred under N₂ at room temperature overnight. Then it was concentrated in vacuo. The residue was purified via column chromatography (silica gel, 5% EtOAc/Hexanes). 3-(N-methyl-N′-tert-butyloxycarbonylhydrazino)-2-(4′-chlorophenyl) propene was obtained as a white solid (0.82 g, 78%). ¹HNMR (CDCl₃, 300 MHz) δ 7.48 (d, J=9 Hz, 2H), 7.26 (d, J=9 Hz, 2H), 5.45 (s, 1H), 5.23 (s, 1H), 3.73 (br s, 2H), 2.60 (s, 3H), 1.40 (s, 9H).

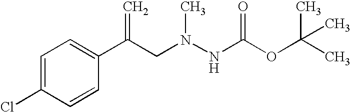

To a mixture of 3-(N-methyl-N′-tert-butyloxycarbonylhydrazino)-2-(4′-chlorophenyl) propene (0.82 g, 2.76 mmol) in MeOH (5 ml) was added a solution of HCl in ether (2 M, 5 ml, 10 mmol). The reaction mixture was stirred under N₂ at room temperature overnight. Then it was concentrated in vacuo. The residue was washed with ether. The solid formed was collected by filtration. N-[2-(4′-chlorophenyl)-allyl]-N-methyl-hydrazine hydrochloride was obtained as a white solid (0.6 g, 94%). Mp: 132-134° C. ¹HNMR (D₂O, 300 MHz) δ 7.27-7.52 (m, 4H), 5.70 (s, 1H), 5.50 (s, 1H), 4.09 (s, 2H), 2.76 (s, 3H).

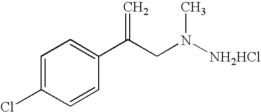

### Example 5

### N-[2-(4′-Chlorophenyl)-allyl]-N-ethyl-hydrazine Hydrochloride

A mixture of 3-(N′-tert-butyloxycarbonylhydrazino)-2-(4′-chlorophenyl) propene (see Example 3) (0.42 g, 1.49 mmole) and N,N-diisopropylethylamine (0.38 g, 2.97 mmol) in DMF (10 ml) was stirred under N₂ at room temperature for 20 min. To this stirred mixture was added EtI (0.46 g, 2.97 mmol). The resulting mixture was stirred under N₂ at room temperature for 4 days. Then it was concentrated in vacuo. The residue was purified via column chromatography (silica gel, 5% EtOAc/Hexanes). 3-(N-ethyl-N′-tert-butyloxycarbonylhydrazino)-2-(4′-chlorophenyl) propene was obtained as an oil (0.38 g, 83%). ¹HNMR (CDCl₃, 300 MHz) δ 7.26-7.52 (m, 4H), 5.45 (s, 1H), 5.25 (s, 1H), 3.77 (br s, 2H), 2.80 (br s, 2H), 1.38 (s, 9H), 1.04 (t, J=6.3 Hz, 3H).

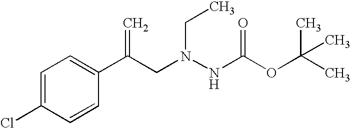

To a mixture of 3-(N-ethyl-N′-tert-butyloxycarbonylhydrazino)-2-(4′-chlorophenyl) propene (0.38, 1.221) in MeOH (3 ml) was added a solution of HCl in ether (2 M, 4 ml, 8 mmol). The reaction mixture was stirred under N₂ at room temperature overnight. Then it was concentrated in vacuo. The residue was washed with ether. The solid formed was collected by filtration. N-[2-(4′-chlorophenyl)-allyl]-N-ethyl-hydrazine hydrochloride was obtained as a white solid (0.27, 90%). Mp: 150-151° C. ¹HNMR (D₂O, 300 MHz) δ 7.25-7.46 (m, 4H), 5.68 (s, 1H), 5.50 (s, 1H), 4.11 (s, 2H), 3.07 (q, J=7.2 Hz, 2H), 1.12 (t, J=7.2 Hz, 3H).

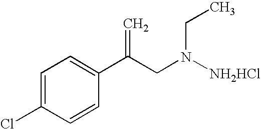

### Example 6

### N-[2-(4′-Chlorophenyl)-allyl]-N,N′-dimethylhydrazine Hydrochloride

To a solution of 3-(N′-tert-butyloxycarbonylhydrazino)-2-(4′-chlorophenyl) propene (see Example 3) (0.42 g, 1.49 mmol) in DMF (10 ml) was added sodium hydride (0.11 g, 4.47 mmol). The mixture was stirred under N₂ at room temperature for 20 min. Then, MeI (0.63 g, 4.47 mmol) was added in one portion. The resulting mixture was stirred under N₂ at room temperature overnight. It was concentrated in vacuo. The residue was purified via column chromatography (silica gel, 5% EtOAc/Hexanes). 3-(N,N′-dimethyl-N′-tert-butyloxycarbonylhydrazino)-2-(4′-chlorophenyl) propene, a colorless oil, was obtained (0.29 g, 63%). ¹HNMR (CDCl₃, 300 MHz) δ 7.46 (d, J=8.7, 2H), 7.26 (d, J=8.7 Hz, 2H), 5.43 (s, 1H), 5.22 (s, 1H), 3.75 (br s, 2H), 2.73 (s, 3H), 2.59 (br s, 3H), 1.43 (s, 9H).

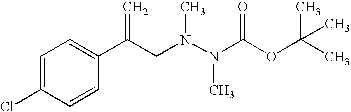

To a mixture of 3-(N,N′-dimethyl-N′-tert-butyloxycarbonylhydrazino)-2-(4′-chlorophenyl) propene (0.29 g, 0.93 mmol) in MeOH (3 ml) was added a solution of HCl in ether (2 M, 4 ml, 8 mmol). The resulting mixture was stirred under N₂ at room temperature overnight. Then it was concentrated in vacuo. The residue was washed with ether. The solid formed was collected by filtration. N-[2-(4′-chlorophenyl)-allyl]-N,N′-dimethylhydrazine hydrochloride was obtained as a white solid (0.17 g, 74%). Mp: 108-110° C. ¹HNMR (D₂O, 300 MHz) δ 7.27-7.56 (m, 4H), 5.60 (s, 1H), 5.42 (s, 1H), 3.93 (s, 2H), 2.70 (s, 3H), 2.58 (s, 3H).

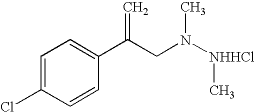

### Example 7

### N-[2-(4′-fluorophenyl)-allyl]-N′-methylhydrazine Hydrochloride

To a mixture of 4-fluoro-α-methylstyrene (13.62 g, 100 mmol) and NBS (21.36 g, 120 mmol) in CH₂Cl₂/THF (4:1, 50 ml) was added Yb(OTf)₃ (3.1 g, 5 mmol) and 5 mol % TMSCl (0.54 g). The resulting mixture was stirred at room temperature for 2 hrs. TLC showed that the starting material disappeared. The reaction mixture was concentrated in vacuo. The residue was purified via column chromatography (silica gel, 100% hexanes) to yield 4-fluoro-α-bromomethylstyrene as an oil (13.54 g, 63%).

¹HNMR (CDCl₃, 300 MHz) δ 7.47 (br s, 2H), 7.37 (br s, 2H), 5.50 (s, 1H), 5.47 (s, 1H), 4.36 (s, 2H).

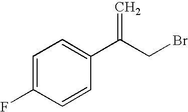

To a mixture of di-tert-butylhydrazodiformate (9.29 g, 40 mmol) and NaH (0.96 g, 40 mmol) in DMF (40 ml) was added 4-fluoro-α-bromomethylstyrene (6.45 g, 30 mmol). The resulting reaction mixture was stirred under N₂ at room temperature and monitored by TLC. When TLC showed that the reaction was completed, it was concentrated in vacuo. The residue was purified via column chromatography (silica gel, 0-5% EtOAc/hexanes). 3-(N,N′-di-tert-butyloxycarbonylhydrazino)-2-(4′-fluorophenyl) propene was obtained as an oil (8.33 g, 83%). ¹H NMR (CDCl₃, 300 MHz) δ 7.42 (br s, 2H), 7.01 (br s, 2H), 5.42 (br s, 1H), 5.17 (s, 1H), 4.50 (s, 2H), 1.43 (s, 18H).

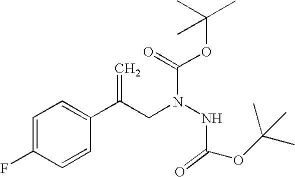

A mixture of 3-(N,N′-di-tert-butyloxycarbonylhydrazino)-2-(4′-fluorophenyl) propene (1.0 g, 2.73 mmol) and NaH (0.11 g, 4.55 mmol) in DMF (30 ml) was stirred under N₂ at room temperature for 20 min. To this mixture was added dropwise MeI (0.65 g, 4.55 mmol). The resulting mixture was stirred under N₂ at room temperature for overnight. It was concentrated in vacuo. The residue was purified via column chromatography (silica gel, 0-5% EtOAc/hexanes), which afforded an oil (1.12 g, ). ¹H NMR (CDCl₃, 300 MHz) δ 7.48 (br s, 2H), 7.03 (br s, 2H), 5.49 (s, 1H), 5.20 (s, 1H), 4.10 (br s, 2H), 2.75 (s, 3H), 1.45 (s, 18H).

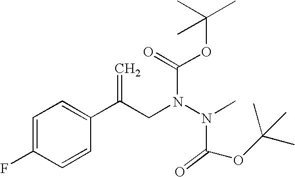

To a mixture of 3-(N,N′-di-tert-butyloxycarbonyl-N′-methylhydrazino)-2-(4′-fluorophenyl) propene (1.12 g, 2.94 mmol) in MeOH (4.0 ml) was added a solution of HCl in ether (2M, 6.0 ml, 12 mmol). The resulting mixture was stirred under N₂ at room temperature overnight. The solvent and excess HCl were removed in vacuo. The residue was washed with ether several times, then dried to give N-[2-(4′-fluorophenyl)-allyl]-N′-methylhydrazine hydrochloride as a white solid (0.56 g, 88%). Mp: 128-129° C.

¹H NMR (D₂O, 300 MHz) δ 7.41 (br s, 2H), 7.03 (br s, 2H), 5.50 (s, 1H), 5.29 (s, 1H), 3.95 (s, 2H), 2.67 (s, 3H).

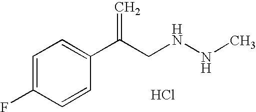

### Example 8

### N-[2-(4′-fluorophenyl)-allyl]-hydrazine Hydrochloride

A mixture of tert-butyl carbazate (6.61 g, 50 mmole) and Et₃N (5.06 g, 50 mmole) in MeOH (40 ml) was stirred at room temperature for 20 min. To this stirred mixture was added 4-fluoro-α-bromomethylstyrene (see Example 7) (6.45 g, 30 mmol). The resulting mixture was heated to reflux and monitored by TLC. TLC showed that the reaction was completed after refluxing for 3 hrs. The mixture was concentrated in vacuo. The residue was purified via column chromatography (silica gel, 5-10% EtOAc/Hexanes). 3-(N′-tert-butyloxycarbonylhydrazino)-2-(4′-fluorophenyl) propene was obtained as a white solid (1.9 g, 24%). ¹H NMR (CDCl₃, 300 MHz) δ 7.42-7.52 (m, 2H), 7.02 (t, J=8.4 Hz, 2H), 5.43 (s, 1H), 5.27 (s, 1H), 3.87 (s, 2H), 1.47 (s, 9H).

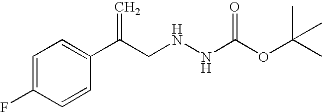

A mixture of 3-(N′-tert-butyloxycarbonylhydrazino)-2-(4′-fluorophenyl) propene (0.8 g, 3.0 mmol) and HCl in ether (2.0 M, 5.0 ml, 10 mmol) in MeOH (4.0 ml) was stirred under N₂ at room temperature overnight. It was concentrated in vacuo. The residue, a white solid, was washed several times with ether, collected by filtration, then dried. N-[2-(4′-fluorophenyl)-allyl]-hydrazine hydrochloride was obtained as a white solid (0.55 g, 83%). Mp: 149-150° C. ¹H NMR (D₂O, 300 MHz) δ 7.35-7.46 (m, 2H), 7.05 (t, J=8.4 Hz, 2H), 5.58 (s, 1H), 5.38 (s, 1H), 4.04 (s, 2H).

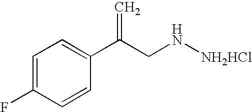

### Example 9

### (2-methyl-allyl)hydrazine Hydrochloride

A mixture of tert-butyl carbazate (1.72 g, 13 mmol) and Et₃N (1.81 ml, 13 mmol) in MeOH (25 ml) was stirred at room temperature for 20 min. To this stirred mixture was added 3-bromo-2-methylpropene (1.26 ml, 12.5 mmol). The resulting mixture was heated to reflux and monitored by TLC. TLC showed that the reaction was completed after refluxing for 3 hrs. The mixture was concentrated in vacuo. The residue was purified on column chromatography (silica gel, 20% EtOAc/hexanes) to give 3-(N′-tert-butyloxycarbonylhydrazino)-2-methyl-propene as an oil (0.7 g, 30%). ¹H NMR (CDCl₃, 300 MHz) δ 4.92 (br s, 2H), 3.43 (s, 2H), 1.78 (s, 3H), 1.46 (s, 9H).

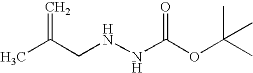

To a solution of 3-(N′-tert-butyloxycarbonylhydrazino)-2-methyl-propene (0.7 g, 3.76 mmol) in MeOH (5 ml) was added a solution of HCl in 1,4-dioxane (4M, 3.8 ml, 15.2 mmol). The resulting mixture was stirred under N₂ at room temperature overnight. Then it was concentrated in vacuo to give a solid. The solid was washed with ether and EtOAc, then dried. A white solid (0.4 g, 87%) was obtained. ¹H NMR (D₂O, 300 MHz) δ 5.05 (s, 1H), 4.95 (s, 1H), 3.53 (s, 2H), 1.65 (s, 3H).

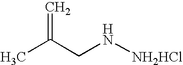

### Example 10

### (E)-1-fluoro-2-phenyl-3-hydrazinopropene Hydrochloride

To a cooled solution of (E)-2-phenyl-3-fluoroallyl alcohol (synthesized by using the procedures described in J. Med. Chem. McDonald; I. A. et al. (1985), 28, 186-193) (1.1 g, 7.47 mmol), N-tert-butyloxycarbonylaminophthalimide (prepared according to the procedures described in J. Org. Chem. Brosse; N. et al. (2000), 65, 4370-4374) (1.95 g, 7.47 mmol), and PPh₃ (2.94 g, 11.22 mmol) in THF (120 ml) was added DEAD (1.8 ml, 11.09 mmol) in one portion. The resulting mixture was stirred under N₂ at room temperature overnight. Then, it was concentrated in vacuo. The residue was triturated in EtOAc, and filtered. The filtrate was concentrated in vacuo. The residue was purified via column chromatography (silica gel, 10% EtOAc/hexanes) to give an oil (1.3 g, 44%).

¹H NMR (CDCl₃, 300 MHz) δ 7.71-7.95 (m, 5H), 7.15-7.55 (m, 4H), 6.81 (2d, J=81.3 Hz, 1H), 4.64, 4.55 (2 s, 2H), 1.44, 1.30 (2 s, 9H).

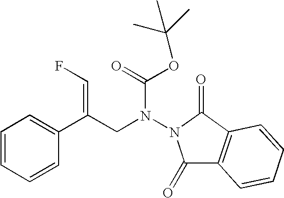

A mixture of N-[(E)-2-phenyl-3-fluoroallyl]-N-tert-butyloxycarbonylamino-phthalimide (1.3 g, 3.28 mmol), H₂NNHMe (0.26 ml, 4.72 mmol) in THF (50 ml) was stirred under N₂ at room temperature for 24 hrs, then was concentrated in vacuo. The residue was washed with EtOAc. A white solid was formed. It was filtered and washed with EtOAc. The filtrate was concentrated to give a semi solid (0.90 g). ¹H NMR (CDCl₃, 300 MHz) δ 7.21-7.53 (m, 5H), 6.78 (d, J=83.1 Hz, 1H), 4.26 (s, 2H), 1.40 (s, 9H). It was used directly in the next step without any further purification.

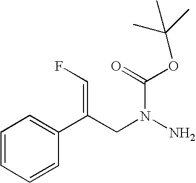

A mixture of 1-[(E)-2-phenyl-3-fluoroallyl]-1-tert-butyloxycarbonylhydrazine (0.98 g, 3.27 mmol) and 4 M HCl in 1,4-dioxane (4.0 ml, 16 mmol) in MeOH (5 ml) was stirred under N₂ at room temperature overnight. The mixture was concentrated in vacuo. The residue was washed several times with ether. The solid formed was collected by filtration, and dried to give (E)-1-fluoro-2-phenyl-3-hydrazinopropene hydrochloride as a white solid (0.35 g, 53%). Mp: 139-141° C. ¹H NMR (D₂O, 300 MHz) δ 7.22-7.47 (m, 5H), 6.96 (d, J=81.0 Hz, 1H), 3.89 (s, 2H).

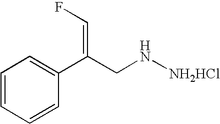

### Example 11

### 2-Aminooxyl-1-phenyl-ethanol Hydrochloride

A mixture of HONHBoc (5.59 g, 42 mmol) and NaOH (1.7 g, 42 mmol) in MeOH (15 ml) was stirred at room temperature for 1 hr. Then, to this stirred solution was added dropwise a solution of styrene oxide (2.52 g, 21 mmol) in MeOH (3 ml). The resulting mixture was heated to keep gentle reflux and monitored by TLC. After refluxing for 3 hrs, TLC showed that the reaction was completed. The reaction mixture was concentrated in vacuo. The residue was diluted with H₂O, extracted with EtOAc (3×20 ml). The combined organic layers were dried (MgSO₄), and then filtered. The filtrate was concentrated in vacuo. The residue was purified via column chromatography (silica gel, 15% EtOAc/hexanes), which afforded 2-(N-tert-butyloxycarbonylaminooxyl)-1-phenylethanol (0.95 g, 18%). mp 97-99° C. ¹H NMR (CDCL₃, 300 MHz) δ 7.25-7.35 (m, 5H), 4.99 (dd, J=9.9, 2.4 Hz, 1H), 3.95 (dd, J=11.7, 9.0 Hz, 1H), 3.77 (dd, J=11.7, 9.9 Hz, 1H), 1.52 (s, 9H).

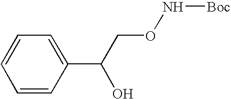

To a solution of 2-(N-tert-butyloxycarbonylaminooxyl)-1-phenylethanol (100 mg, 0.395 mmol) in ether (5 ml) was added a solution of 1M HCl in ether (2.0 ml, 2 mmol). The reaction mixture was stirred under N₂ for 3 hrs at room temperature. A solid was formed and precipitated. The solid was collected by filtration, washed with ether, and then dried in vacuo, to give 2-aminooxyl-1-phenyl-ethanol as a white crystalline solid (40 mg, 53%). Mp 131-132° C. ¹H NMR (D₂O, 300 MHz) δ 7.30 (m, 5H), 4.99 (t, J=5.7 Hz, 1H), 4.10 (d, J=5.4 Hz, 2H).

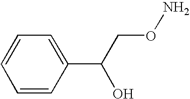

### Example 12

### 2-Aminooxy-1-(3′,4′-dimethoxyphenyl)-ethanol Hydrochloride

To a cooled solution of NaH (0.25 g, 10.42 mmol) in THF (20 ml) was added dropwise a solution of trimethylsulfonium iodide (2.08 g, 10 mmol) in DMSO (20 ml). The resulting mixture was stirred at 0° C. under N₂ for 10 min before a solution of 3,4-dimethoxybenzaldehyde (1.66 g, 10.00 mmol) in THF (5 ml) was added. The resulting reaction mixture was stirred at 0° C. under N₂ for 30 min, then it was warmed gradually to room temperature and stirred at room temperature for 1 hr. The reaction mixture was poured into icewater. The mixture was extracted with hexanes (3×30 ml). The combined organic layers were washed with H₂O, brine, then dried (MgSO₄) and filtered. The filtrate was concentrated in vacuo to afford 2-(3′,4′-dimethoxyphenyl)-oxirane as an oil (1.56 g, 87%). ¹H NMR (CDCl₃, 300 MHz) δ 2.75-2.83 (m, 1H), 3.08-3.17 (m, 1H), 3.76-3.85 (m, 1H), 3.87 (br s, 6H), 6.51 (s, 1H), 6.75-6.91 (m, 2H).

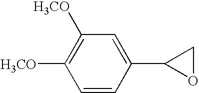

A mixture of HONHBoc (2.31 g, 17.35 mmol) and NaOH (0.69 g, 17.25 mmol) in MeOH(20 ml) was stirred at room temperature for 1 hr. Then, to this stirred solution was added dropwise a solution of 2-(3,4-Dimethoxy-phenyl)-oxirane (1.56 g, 8.67 mmol) in MeOH (3 ml). The resulting mixture was gently refluxed and monitored by TLC. After refluxing for 3 hrs, TLC showed that the reaction was completed. The reaction mixture was concentrated in vacuo. The residue was diluted with H₂O (50 ml), extracted with EtOAc (3×20 ml). The combined organic layers were dried (MgSO₄), and then filtered. The filtrate was concentrated in vacuo. The residue was purified via column chromatography (silica gel, 20-40% EtOAc/hexanes), which afforded 2-(N-tert-butyloxycarbonylaminooxyl)-1-(3′,4′-dimethoxyphenyl)ethanol (0.4 g, 15%). ¹H NMR (CDCl₃, 300 MHz) δ 7.51 (s, 1H), 7.01 (s, 1H), 6.81-6.95 (m, 2H), 4.99 (d, J=3.0 Hz, 1H), 3.97 (s, 3H), 3.75 (s, 3H), 3.70-3.95 (m, 2H), 1.52 (s, 9H).

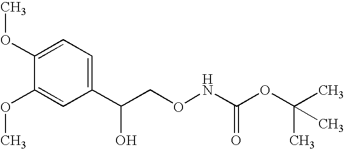

To a solution of 2-(N-tert-butyloxycarbonylaminooxyl)-1-(3′,4′-dimethoxyphenyl)ethanol (80 mg, 0.26 mmol) in CH₂Cl₂ (2 ml) was added a solution of 4M HCl in 1,4-dioxane (1.5 ml, 6.0 mmol). The reaction mixture was stirred under N₂ at room temperature for overnight. A solid was formed and precipitated. The solid was collected by filtration, washed with ether, and then dried in vacuo, which gives 2-aminooxyl-1-(3′,4′-dimethoxyphenyl)-ethanol as a white crystalline solid (50 mg, 55%). mp 100-101° C. ¹H NMR (D₂O,300 MHz) δ 6.93 (s, 1H), 6.85-6.91 (m, 2H), 4.87 (t, J=5.7 Hz, 1H), 4.05 (d, J=5.4 Hz, 2H), 3.70 (s, 3H), 3.68 (s, 3H).

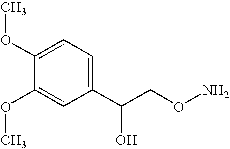

### Example 13

### 2-phenyl-2-cyclopropyl Ethylamine

To a cooled solution of 2-phenylacetonitrile (5.85 g, 50 mmol) in THF (200 ml) was added potassium bis(trimethylsiylyl)amide (29.92 g, 150 mmol). The resulting mixture was stirred under N₂ at 0° C. for 30 min. To the resulting mixture was added dropwise a solution of 1,2-dibromoethane (10.33 g, 55 mmol) in THF (30 ml). The reaction mixture was stirred under N₂ at 0° C. and gradually allowed to warm to room temperature. Then it was stirred at room temperature overnight. It was concentrated in vacuo. The 1-phenyl-1-cyclopropanecarbonitrile was obtained by distillation (3.6 g, 50%). ¹H NMR (CDCl₃, 300 MHz) δ 7.27-7.38 (m, 5H), 1.69-1.76 (m, 2H), 1.37-1.44 (m, 2H).

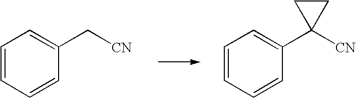

1-phenyl-1-cyclopropanecarbonitrile (1.0 g, 6.98 mmol) was added to a stirred suspension of lithium aluminum hydride (0.27 g, 6.98 mmol) in ether (25 ml). The resulting suspension was refluxed for 2 hrs, them cooled to 0° C. by applying an external ice bath. The excess hydride was quenched by careful addition of H₂O. The resulting mixture was filtered. The solid was washed with ether and filtered. The filtrate was dried (MgSO₄), and filtered. The filtrate was concentrated in vacuo to give 2-cyclopropyl-2-phenylethylamine. ¹H NMR (DMSO-d₆, 300 MHz) δ 7.28 (m, 4H), 7.17 (m, 1H), 2.70 (s, 2H), 1.45 (br s, 2H), 0.78 (dd, J=6.2, 3.8H, 2H), 0.67 (dd, J=6.2, 3.8 Hz, 2H).

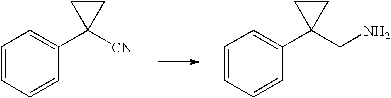

### Example 14

### In Vitro Inhibition of SSAO Activity

SSAO activity was measured using the coupled colorimetric method essentially as described for monoamine oxidase and related enzymes (Holt A. et al. (1997) Anal. Biochem. 244:384). Bovine plasma amine oxidase (PAO) was purchased from Worthington Biochemical (Lakewood, N.J.) and used as a source of SSAO for activity measurements. The SSAO assay was performed in 96 well microtitre plates as follows. A pre-determined amount of inhibitor diluted in 0.2 M potassium phosphate buffer, pH 7.6, was added to each well, if required. The amount of inhibitor varied in each assay but was generally at a final concentration of between 10 nM and 10 μM. Controls lacked inhibitor. In order to study the effects of potential inhibitors 50 μl of inhibitor solution were preincubated for 30 min at 37° C. with 0.4 mU of PAO in an a total volume of 130 μl of 0.2 M potassium phosphate buffer pH 7.6. Assays were then started by addition of 20 μl 10 mM benzylamine substrate and incubated for 20 min at 37° C. The following reagents were then added to a final reaction volume of 200 μl, 50 μl of freshly made chromogenic solution containing 750 nM vanillic acid (Sigma # V-2250), 400 nM 4-aminoantipyrine (Sigma # A-4328) and 12 U/ml horseradish peroxidase (Sigma # P-8250) in order to cause a change of 0.5 OD A490 per hour. This was within the linear response range of the assay. The plates were incubated for 1 hr at 37° C. and the increase in absorbance, reflecting SSAO activity, was measured at 490 nm using a microplate spectrophotometer (Power Wave 40, Bio-Tek Inst.). Inhibition was presented as percent inhibition compared to control after correcting for background absorbance and IC₅₀ values calculated using GraphPad Prism software.

SSAO activity was also measured as described (Lizcano J M. Et al. (1998) Biochem J. 331:69). Briefly, rat lung homogenates were prepared by chopping the freshly removed tissue into small pieces and washing them thoroughly in PBS. The tissue was then homogenized 1:10 (w/v) in 10 mM potassium phosphate buffer (pH 7.8) and centrifuged at 1000 g at 4° C. for 10 minutes; the supernatant was kept frozen until ready to use. SSAO activity in 100 ul of lung homogenate was determined radiochemically using 20 uM ¹⁴C-benzylamine as substrate. The reaction was carried out at 37° C. in a final volume of 300 ul of 50 mM potassium phosphate buffer (pH 7.2) and stopped with 100 ul of 2 M citric acid. Radioactively labeled products were extracted into toluene/ethyl acetate (1:1, v/v) containing 0.6% (w/v) 2,5-diphenyloxdazole (PPO) before liquid scintillation counting. Using this method the inhibitory activity of the compounds of Examples 2 and 8 was also tested in the presence of up to 50% human serum. There were no changes to the SSAO IC₅₀ values of both compounds in the presence of serum.

### Example 15

### Comparison of Inhibition of the SSAO Activity of SSAO/VAP-1 Versus MAO-A and MAO-B Activities

The specificity of the different SSAO inhibitors was tested by determining their ability to inhibit MAO-A and MAO-B activities in vitro. Recombinant human MAO-A and human MAO-B enzymes were obtained from BD Biosciences (MA, USA). MAO activities were measured in a similar way as for SSAO except that no pre-incubation with inhibitor or substrate was performed. A pre-determined amount of inhibitor diluted in 0.2 M potassium phosphate buffer, pH 7.6, was added to each well, if required. The amount of inhibitor varied in each assay but was generally at a final concentration of between 50 nM and 1 mM. Controls lacked inhibitor. The following agents were then added to a final reaction volume of 200 μl in 0.2 M potassium phosphate buffer, pH 7.6: 0.04 mg/ml of MAO-A or 0.07 mg/ml MAO-B enzyme, 15 μl of 10 mM tyramine substrate (for MAO-A), or 15 μl 100 mM benzylamine substrate (for MAO-B), and 50 μl of freshly made chromogenic solution (as above). The plates were incubated for 60 min at 37° C. The increase in absorbance, reflecting MAO activity, was measured at 490 nm using microplate spectrophotometer (Power Wave 40, Bio-Tek Inst.). Inhibition was presented as percent inhibition compared to control after correcting for background absorbance and IC₅₀ values calculated using GraphPad Prism software. Clorgyline and pargyline (inhibitors of MAO-A and -B, respectively) at 0.5 and 10 μM, respectively, were added to some wells as positive controls for MAO inhibition. The ability of compounds of the previous Examples to inhibit SSAO activity versus MAO activity is shown in Table 1. The results show that the compounds described in the present invention are specific inhibitors of SSAO activity. The compounds described in the present invention are therefore expected to have therapeutic utility in the treatment of diseases and conditions in which the SSAO activity of SSAO/VAP-1 plays a role, that is, in SSAO/VAP-1 mediated diseases and conditions.

TABLE 1
Potency and specificity of Examples 1 to 12

| Ex- | SSAO | MAO-A | MAO-B | | |
|---|---|---|---|---|---|
| ample | Inhibitory | Inhibitory | Inhibitory | Specificity | Specificity |
| Com- | Activity | Activity | Activity | for SSAO | for SSAO |
| pound | IC₅₀ (μM) | IC₅₀ (μM) | IC₅₀ (μM) | vs. MAO-A | vs. MAO-B |
| 1 | 0.029 | 900 | 125 | 31,000 | 4,300 |
| 2 | 0.035 | 225 | 100 | 6,400 | 2,900 |
| 3 | 0.050 | 1 | 24.5 | 20 | 490 |
| 4 | 0.65 | 260 | 175 | 400 | 269 |
| 5 | 0.95 | 690 | 155 | 726 | 163 |
| 6 | 4.7 | 300 | 2.8 | 64 | 0.60 |
| 7 | 2.61 | 620 | 210 | 230 | 80 |
| 8 | 0.032 | 2.2 | 81 | 69 | 2,500 |
| 9 | 0.055 | 350 | 20 | 6,300 | 360 |
| 10 | 0.065 | 2.8 | 0.65 | 43 | 10 |
| 11 | 0.050 | 250 | 450 | 5000 | 9000 |
| 12 | 0.090 | 3,6000 | 8,200 | 400,000 | 90,000 |

### Example 16

### Acute Toxicity Studies

Intraperitoneal (i.p.) and intravenous (i.v.) LD₅₀ values for the compounds of Examples 8 and 10, as well as mofegiline, the allylamine compound described in Example 18, were determined in mice. Six-week old C57B1/6 female mice were divided in groups of five and administered a single i.p or i.v. injection of compound dissolved in PBS (10-100 mg/kg in 100 ul i.v.; 30-500 mg/kg in 200 ul i.p.). Control groups were administered the same volume of PBS i.p. or i.v. Appearance and overt behavior were noted daily, and body weight was measured before compound administration (Day 1) and on Days, 3, 5 and 7. After seven days, animals were euthanized and their liver, spleen and kidneys weighted. The results of the acute toxicity study are summarized in Table 2.

TABLE 2
Intraperitoneal and intravenous LD₅₀ (mg/kg)* values
for mofegiline and compounds of Examples 8 and 10.

| Mode of | | | |
|---|---|---|---|
| Administration | Mofegiline | Example 8 | Example 10 |
| intraperitoneal | 200 | 350 | 250 |
| intravenous | 70 | >100 | >100 |

*Numbers represent the LD₅₀ Day 7 values.

Acute toxicity effects for mofegiline included tremors (40 mg/kg i.v.; 100 mg/kg i.p.) and clonic convulsions and labored breathing (100 mg/kg i.v.; 200 mg/kg i.p.). Mice receiving 100 mg/kg i.v. of the compounds of Examples 8 and 10 exhibited only tremor, whereas convulsions and labored breathing were only observed at doses greater than about 300 mg/kg for both compounds (see Table 2; greater than about 250 mg/kg for the compound of Example 10, greater than about 350 mg/kg for the compound of Example 8). All deaths occurred within 24 hours after drug administration. Postmortem examinations did not reveal any gross lesions. Body weights as well as absolute and body weight-normalized organ weights were not significantly different from the control group for any of the compounds (p>0.05 by Dunnett's test following analysis of variance). Thus, there is no indication of the cause of death for any of the compounds tested. However, as indicated by the much higher levels of the compounds of Examples 8 and 10 required to induce tremors, convulsions, and labored breathing, compounds of the current invention are significantly less toxic than mofegiline.

### Example 17

### Inhibition of Collagen-Induced Arthritis in Mice

Collagen-induced arthritis (CIA) in mice is widely used as an experimental model for rheumatoid arthritis (RA) in humans. CIA is mediated by autoantibodies to a particular region of type II collagen and complement. The murine CIA model used in this study is called antibody-mediated CIA, and can be induced by i.v. injection of a combination of different anti-type II collagen monoclonal antibodies (Terato K., et al. (1995). Autoimmunity. 22:137). Several compounds have been used to successfully block inflammation in this model, including anti-α1β1 and anti-α2β2 integrins monoclonal antibodies (de Fougerolles A. R. (2000) J. Clin. Invest. 105: 721).

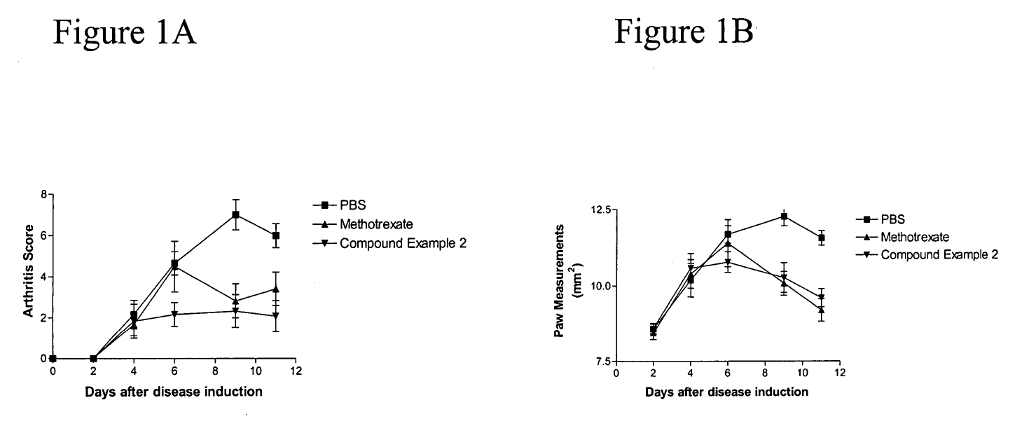

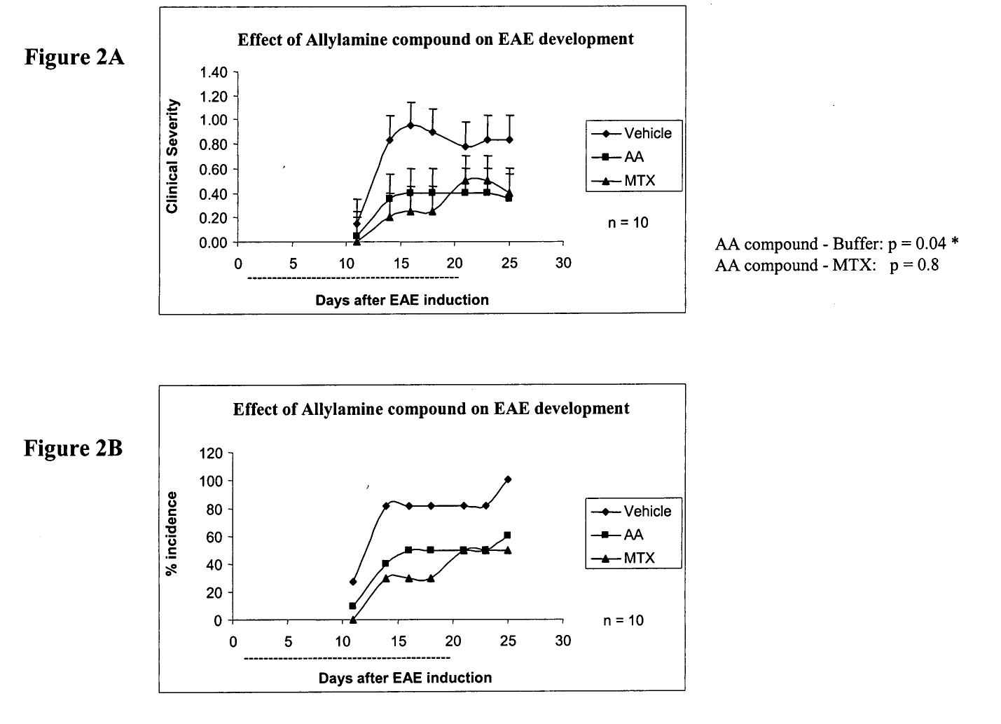

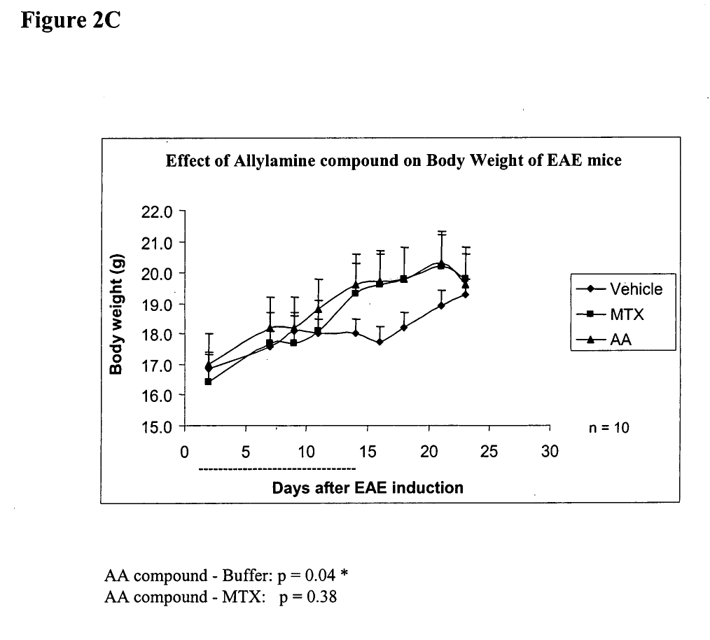

In this example, arthrogen-collagen-induced arthritis antibody kits were purchased from Chemicon International (Temecula, Calif.) and arthritis was induced using the manufacturer's protocol. Mice were injected i.v. with a cocktail of 4 anti-collagen Type II monoclonal antibodies (0.5 mg each) on day 0, followed by i.p. injection of 25 μg lipopolysaccharide (LPS) on day 2. Mice develop swollen wrists, ankles, and digits 3-4 days after LPS injection, with disease incidence of 90% by day 7. Severity of arthritis in each limb was scored for 12 days as follows: 0=normal; 1=mild redness, slight swelling of ankle or wrist; 2=moderate redness and swelling of ankle or wrist; 3=severe redness and swelling of some digits, ankle and paw; 4=maximally inflamed limb. Animals were divided in 3 groups of 6 animals: vehicle, methotrexate (MTX)-treated, and compound-treated. Animals in the vehicle group were injected i.p. with phosphate buffer saline (PBS), twice daily for 12 days (starting on day 0). MTX (3 mg/kg) was administered i.p. starting on day 0 and continuing every other day (Mon., Weds., Fri.) for the duration of the experiment. Administration of the compound of Example 2 (20 mg/kg/dose, i.p., two doses daily) was initiated at day 0 and continued until day 11. The results are shown in FIG. 1A, FIG. 1B, and FIG. 1C. The administration of 20 mg/kg of the compound of Example 2, twice daily, clearly reduced the final arthritis score and paw swelling in this model.

For each of the two sets of data (arthritis score and paw swelling), a repeated measure analysis was performed to assess the treatment effect. For arthritis scores, there is significant overall treatment effect (p=0.0165). There is no significant difference between the compound of example 2 and MTX in terms of treatment effect (p=0.3348). However, the compound of example 2 shows significant treatment effect when compared to PBS vehicle (p=0.0046). For swelling, there is significant overall treatment effect (p=0.0294). There is no significant difference between the compound of example 2 and MTX in terms of treatment effect (p=0.8772). However, the compound of example 2 shows significant treatment effect when compared to PBS (p=0.0060).

Follow-up studies involved looking at cytokine levels in sera and the affected tissues. Whereas circulatory cytokines are easy to measure by ELISA, determination of levels of cytokines in paw, colon, and spinal cord is not as straightforward. The relative RT-PCR approach was used. To perform this experiment, a kit from Ambion (USA) was used that employs 18S ribosomal RNA (rRNA) as an internal control. In addition to the rRNA primers, the kit also provides specific primers for the amplification of the different cytokines to be analyzed. The advantage of using 18S rRNA as a standard is that, as opposed to mRNA for specific genes, it is expressed at static levels across a broad range of tissues and treatment conditions. Still, for each tissue to be analyzed, amplification procedures have to be optimized so that both the gene of interest and the rRNA are in a linear range. At the end of the study, animals were euthanized and the right hind paws were extracted and frozen. Total RNA was isolated using 1 ml of Trizol reagent (Invitrogen, USA) per 50-100 mg tissue according to the manufacturer's instructions. Five micrograms of total RNA was used in the first strand cDNA synthesis following the protocol provided with the Ambion TNF-alpha (mouse) Gene Specific Relative RT-PCR Kit (catalog # 5439) and 2 μl used as template in the qualitative RT-PCR reaction. PCR cycling conditions were as follows: hot start for 2 min at 94° C. followed by 27 cycles of denaturation for 45 sec at 94° C., 45 sec annealing at 50° C. and 45 sec extension at 72° C., and one final extension for 7 min at 72° C. A 10 μl aliquot from each PCR reaction was run in a 6% acrylamide/TBE gel (Invitrogen, USA) and stained with ethidium bromide. FIG. 8A shows the results of one of these experiments, where RNA from the digits, foot pad and ankles of one single animal was amplified using primers for rRNA and mouse TNFα (the paws of this animal had different levels of arthritis score). Quantitative densitometry analysis (Gel Doc 2000 gel documentation system and Quantity One 4.3.1 software, BioRad, USA) of the different bands allowed comparison of the relative TNFα:rRNA ratios between samples. FIG. 8B shows the results obtained when total RNA was isolated from the right hind paw of all animals from the experiment depicted in FIG. 1 and used to determine the relative ratios between 18S and TNFα levels.

Data were analyzed with GraphPad Prism software (San Diego, Calif.) by Dunnett's test following analysis of variance. These results show that the compound of example 2 (used in the experiment in FIG. 1) was able to reduce the levels of TNFα mRNA in the paws of mice with CIA.

### Example 18A

### Inhibition of Experimental Autoimmune Encephalomyelitis in Mice by SSAO Inhibitors—Mofegiline (Allylamine Compound)

SSAO/VAP-1 is expressed on the endothelium of inflamed tissues/organs including brain and spinal cord. Its ability to support lymphocyte transendothelial migration may be an important systemic function of SSAO/VAP-1 in inflammatory diseases such as multiple sclerosis and Alzheimer's disease. An analysis of the use of SSAO inhibitors to treat inflammatory disease of the central nervous system (CNS) was performed through the use of an experimental autoimmune encephalomyelitis model (EAE) in C57BL/6 mice. EAE in rodents is a well-characterized and reproducible animal model of multiple sclerosis in human (Benson J. M. et al. (2000) J. Clin. Invest. 106:1031). Multiple sclerosis is a chronic immune-mediated disease of the CNS characterized by pachy perivenular inflammatory infiltrates in areas of demyelination and axonal loss. As an animal model, EAE can be induced in mice by immunization with encephalitogenic myelin antigens in the presence of adjuvant. The pathogenesis of EAE comprises presentation of myelin antigens to T cells, migration of activated T cells to the CNS, and development of inflammation and/or demyelination upon recognition of the same antigens.

To examine the role of SSAO/VAP-1 as a major regulator of the lymphocyte recruitment to the CNS, mofegiline, an allylamine and SSAO inhibitor, was evaluated in an EAE model.

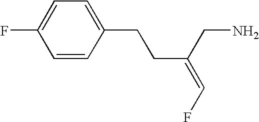

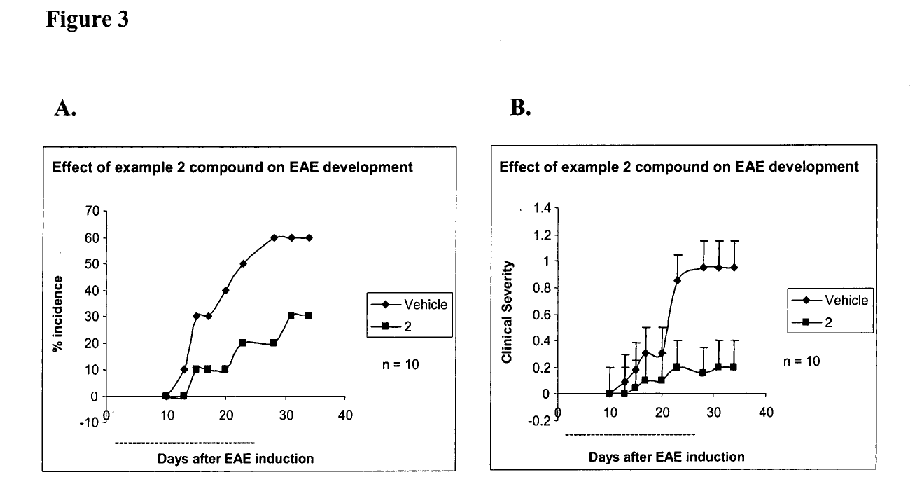

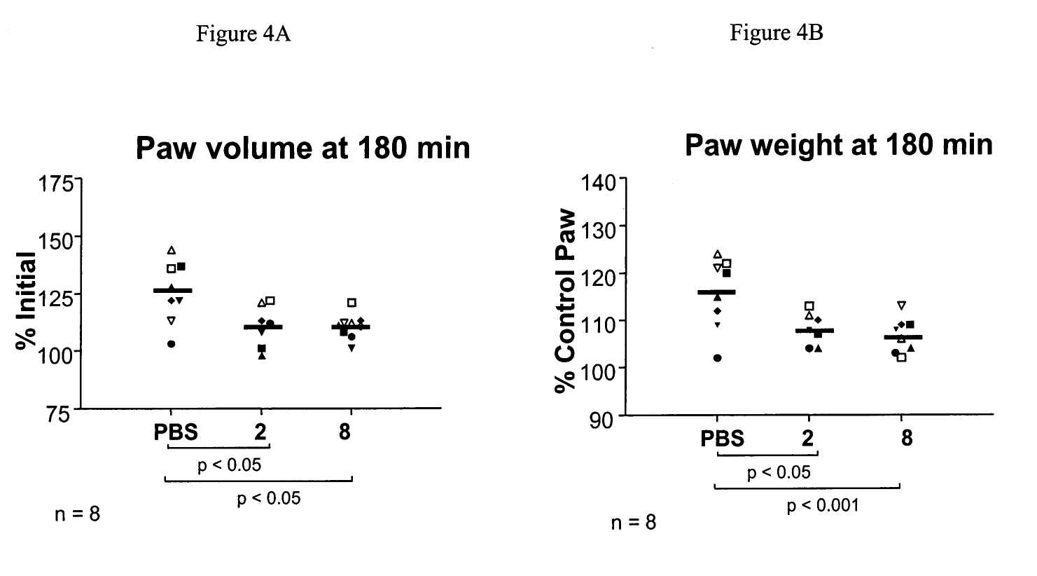

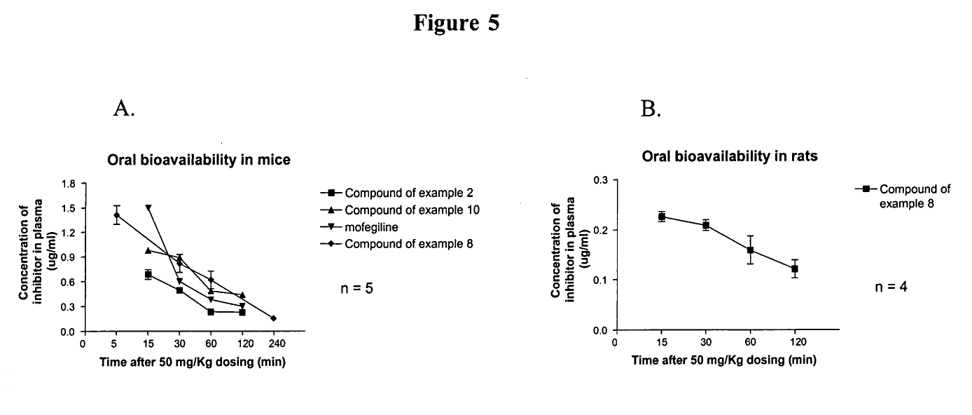

Thirty female C57BL/6 mice were immunized subcutaneously (s.c). with myelin oligodendrocyte glycoprotein 35-55 (MOG peptide 35-55) in Complete Freund Adjuvant (CFA) on day 0, followed by i.p. injections of pertussis toxin (one pertussis toxin injection on day 0, a second pertussis toxin injection on day 2). Groups of 10 mice received either the allylamine compound mofegiline (AA, 10 mg/kg/dose, twice daily for 18 consecutive days), methotrexate (2.5 mg/kg/day, every other day (Mon., Weds., Fri.) till day 18) or vehicle control (twice/day for 18 consecutive days) all-starting from one day after the immunization and all administered i.p. Then animals were monitored for body weight, signs of paralysis and death according to a 0-5 scale of scoring system as follows: 1=limp tail or waddling gait with tail tonicity; 2=waddling gait with limp tail (ataxia); 2.5=ataxia with partial limb paralysis; 3=full paralysis of one limb; 3.5=full paralysis of one limb with partial paralysis of second limb; 4=full paralysis of two limbs; 4.5=moribund; 5=death. Results are shown in FIG. 2A, FIG. 2B, and FIG. 2C. Compared with the vehicle-treated group during the dosing period (up to day 18), that showed an 80% disease incidence and moderate clinical severity, mofegiline-treated mice resulted in a statistically significant reduction of disease severity with 50% of mice affected. (p=0.04 by repeated measure analysis to assess the treatment effect. Proper polynomial transformation, with the spacing corresponding to the collection days, was applied to test the time effect). Statistically significant differences in diseases severity between the AA and vehicle-treated groups, continued even after stopping compound administration and were observed until the end of the study (d25) .

As expected, the loss of body weight is correlated with the clinical severity in vehicle-control mice; and mofegiline treatment also prevented body weight loss in the mice during the dosing period (p=0.04). In addition, the inhibitory effect of mofegiline on the EAE development was continuously observed for at least one more week after the last treatment (d19-25). MTX-treated mice exhibited a similar inhibitory effect during the treatment period (d0-18). However, a rise in disease incidence and severity was observed right after stopping the MTX treatment (FIG. 2A). There was no statistically significant difference (p=0.8 and p=0.38, for clinical severity and body weight, respectively) between the groups treated with MTX and mofegiline during or after the dosing period.

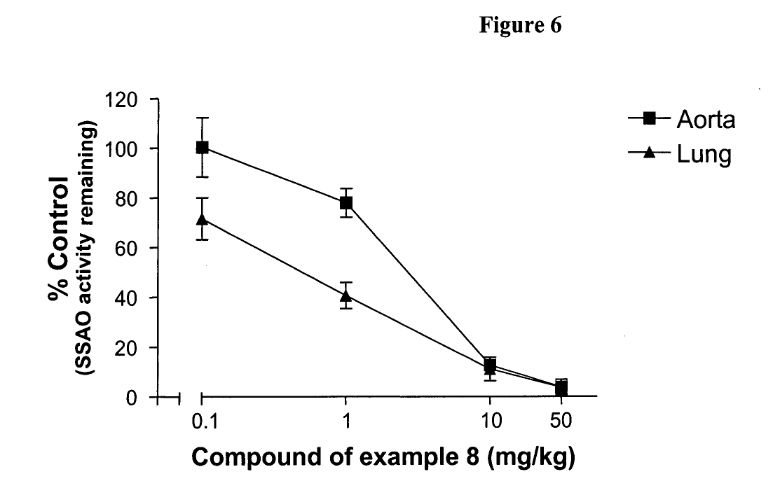

The exact same protocol was followed in a separate experiment using the compound of Example 2, except that this time the MTX group was omitted. The results shown in FIG. 3 indicate that this compound clearly had a therapeutic effect on the development and severity of disease. These data indicate that the compounds of the invention are candidates for treatment of multiple sclerosis in humans.

### Example 18B

### Inhibition of Relapsing Experimental Autoimmune Encephalomyelitis in Mice by VAP-1/SSAO Inhibitor (Model of Chronic Multiple Sclerosis)

An analysis of the use of VAP-1/SSAO inhibitors to treat inflammatory diseases of the CNS is performed through the use of a relapsing experimental autoimmune encephalomyelitis model (EAE) in SJL/J mice. Relapsing EAE in mice is a well-characterized and reproducible animal model of multiple sclerosis in humans (Brown & McFarlin 1981 Lab. Invest. 45:278-284; McRae et al 1992 J. Neuroimmunol. 38:229-240). Multiple sclerosis is a chronic immune-mediated disease of the CNS characterized by pachy perivenular inflammatory infiltrates in areas of demyelination and axonal loss. As an animal model, chronic relapsing EAE can be induced in mice by immunization with encephalitogenic myelin antigen in the presence of adjuvant. The pathogenesis of EAE comprises presentation of myelin antigens to T cells, migration of activated T cells to the CNS, and development of inflammation and/or demyelination upon recognition of the same antigens.

Vascular adhesion protein-1 (VAP-1) is an amine oxidase and adhesion receptor that is expressed on the endothelium of inflamed tissues/organs including brain and spinal cord. Its ability to support lymphocyte transendothelial migration may be an important systemic function of VAP-1 in inflammatory disorders such as multiple sclerosis and Alzheimer's disease.

To examine the role of VAP-1 as a major regulator of lymphocyte recruitment to the CNS, VAP-1/SSAO inhibitor was evaluated in a chronic relapsing EAE model. Twenty 7-8 week old female SJL/J mice were immunized s.c. with 50 μg of mouse PLP peptide 139-151 in Complete Freund Adjuvant (CFA), followed by two i.p. injections of 200 ng pertussis toxin. Groups of 10 mice received i.p. either vehicle control (PBS, 0.1 ml) or (2-phenylallyl)hydrazine at 10 mg/kg, bid for 53 consecutive days, all-starting from one day after the immunization. (2-phenylallyl)hydrazine is the following compound:

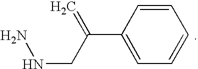

Then animals were monitored for signs of paralysis according to a 0-5 scale of scoring system as follows:
0.5 partial tail weakness
1 limp tail or waddling gait with tail tonicity;
1.5 waddling gait with partial tail weakness
2 waddling gait with limp tail (ataxia);
2.5 ataxia with partial limb paralysis;
3 full paralysis of one limb;
3.5 full paralysis of one limb with partial paralysis of second limb;
4 full paralysis of two limbs;
4.5 moribund;
5 death.

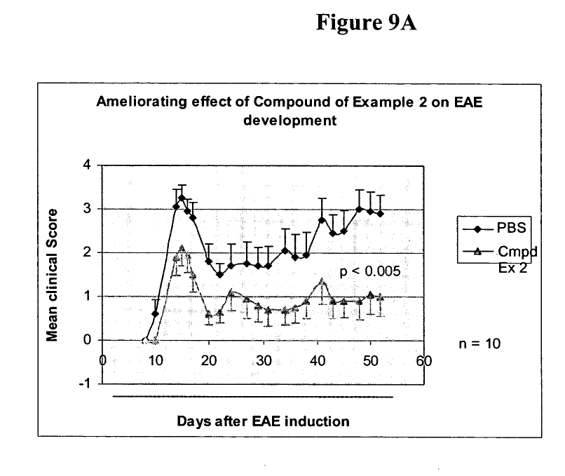

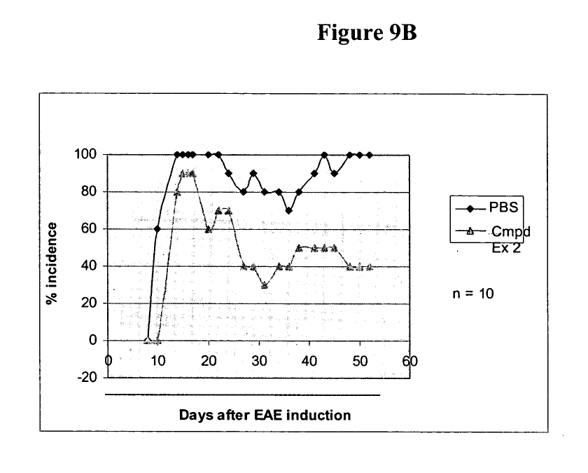

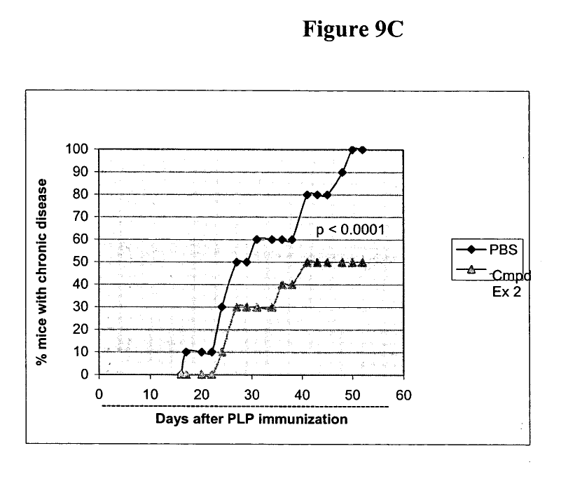

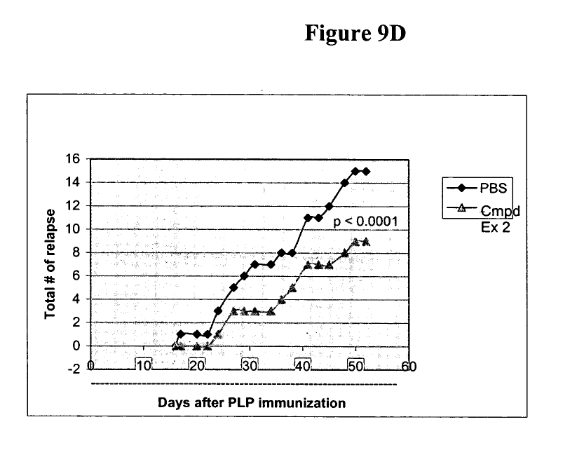

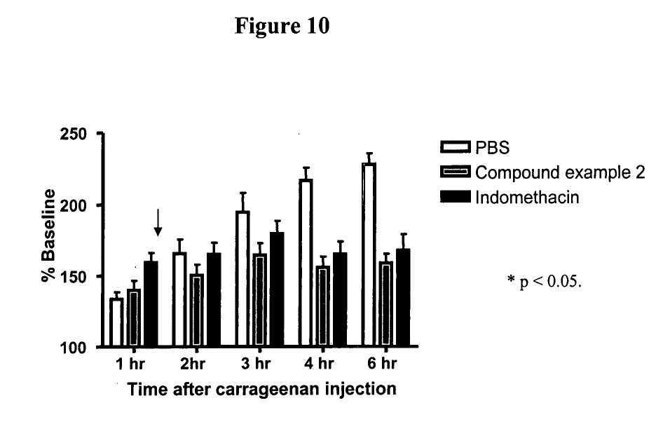

The results are expressed as mean clinical score (FIG. 9A), % incidence (number of mice with any paralysis/10 mice) (FIG. 9B), % mice with chronic disease (mice with at least one relapse) (FIG. 9C), and cumulative total number of relapses (FIG. 9D). The p value for clinical score were analyzed by a repeated measure method, and the p values for both accumulated number of relapses and percent of mice with chronic disease were calculated by a generalized linear model with main effects being treatment group ((2-phenylallyl)hydrazine vs. Buffer) and the day of collection.

As shown in FIG. 9A, FIG. 9B, FIG. 9C, and FIG. 9D, while 90-100% of mice developed moderate to severe paralysis two weeks after the immunization in both groups, the incidence of chronic disease is significant lower (p<0.0001) in the group treated with (2-phenylallyl)hydrazine than in control mice that received buffer. A similar statistically significant reduction in overall clinical severity (p<0.005) and cumulative number of relapses (p<0.0001) was also observed for (2-phenylallyl)hydrazine-treated mice as comparing with the control group that received buffer. Taken together, the results indicate an ameliorating effect of the SSAO/VAP-1 inhibitor on the development of chronic EAE.

### Example 19

### Inhibition of Carrageenan-Induced Rat Paw Edema

Carrageenan-induced paw edema has been extensively used in the evaluation of anti-inflammatory effects of various therapeutic agents and is a useful experimental system for assessing the efficacy of compounds to alleviate acute inflammation (Whiteley P E and Dalrymple S A, 1998. Models of inflammation: carrageenan-induced paw edema in the rat, in Current Protocols in Pharmacology. Enna S J, Williams M, Ferkany J W, Kenaki T, Porsolt R E and Sullivan J P, eds., pp 5.4.1-5.4.3, John Wiley & Sons, New York). The full development of the edema is neutrophil-dependent (Salvemini D. et al. (1996) Br. J. Pharmacol. 118: 829).

Female Sprague Dawley rats were used and compounds of the invention were injected i.p. at 100 mg/kg 15 minutes prior to carrageenan exposure. The control group was injected with an equal volume of vehicle (PBS). Edema in the paws was induced as previously described by injecting 50 μl of a 0.5% solution of carrageenan (Type IV Lambda, Sigma) in saline with a 27-G needle s.c. in the right foot pat. (See Whiteley P. E. and Dalrymple S. A. (1998) Models of inflammation: carrageenan-induced paw edema in the rat, in Current Protocols in Pharmacology. Enna S J, Williams M, Ferkany J W, Kenaki T, Porsolt R E and Sullivan J P, eds., pp 5.4.1-5.4.3, John Wiley & Sons, New York) The size of the tested foot of each animal was measured volumetrically, before induction of edema, and at 60, 120, and 180 min after carrageenan induction.

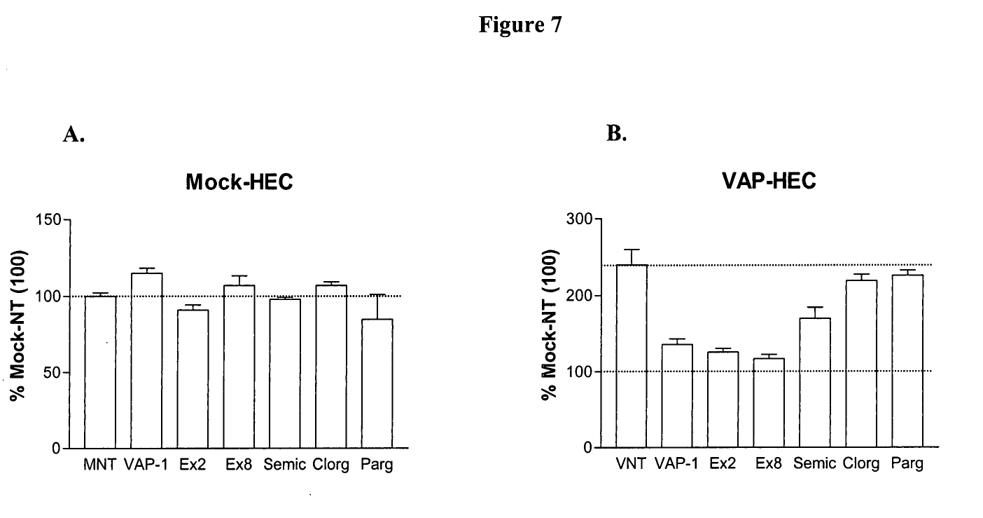

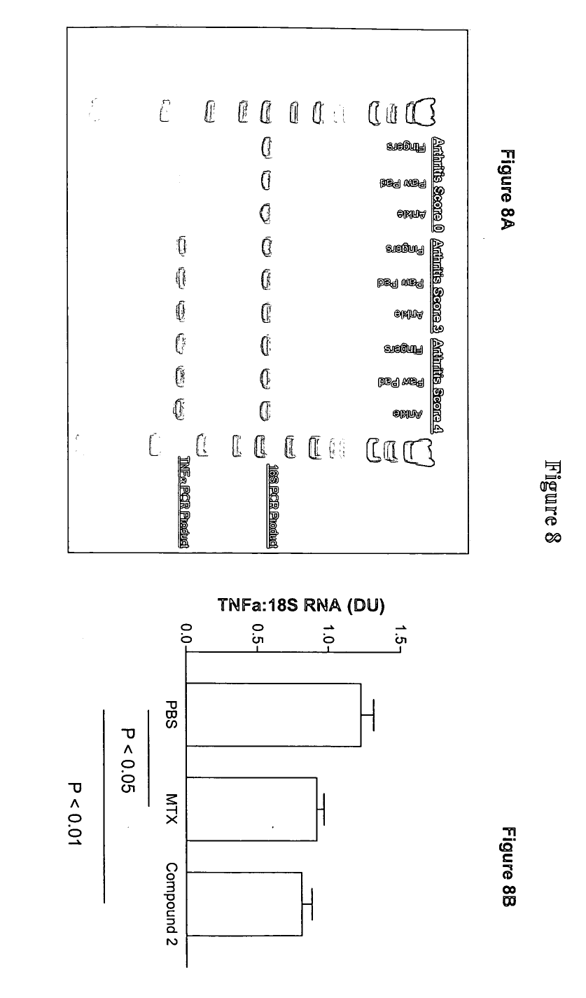

Results of an experiment where the compounds of Examples 2 and 8 were used are shown in FIG. 4. In both cases the 100 mg/kg dose clearly and significantly reduced the paw swelling at all time points tested. Data were analyzed with GraphPad Prism software (San Diego, Calif.) by Dunnett's test following analysis of variance (p<0.05).

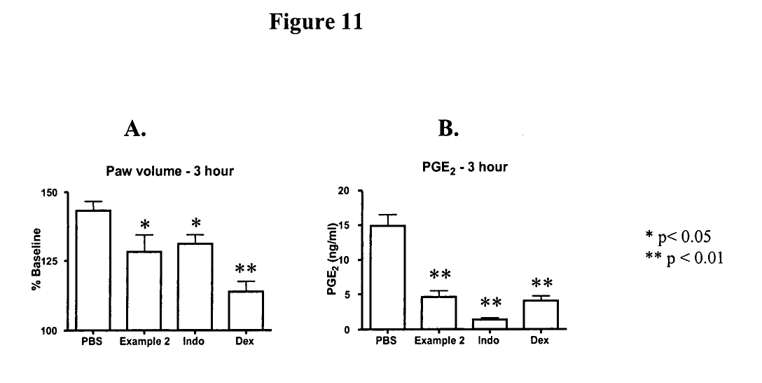

Additional experiments using this model were performed to ascertain whether SSAO inhibitors showed significant efficacy when used in a therapeutic mode (i.e., after injection of carrageenan). Briefly, SSAO inhibitors (30 mg/kg), indomethacin (3 mg/kg) and PBS were administered orally to rats 1 hour after carrageenan injections. Results of one representative experiment are shown in FIG. 10. Data indicate that the SSAO inhibitor tested is able to reduce paw edema when applied in a therapeutic manner to levels comparable to that observed with indomethacin.

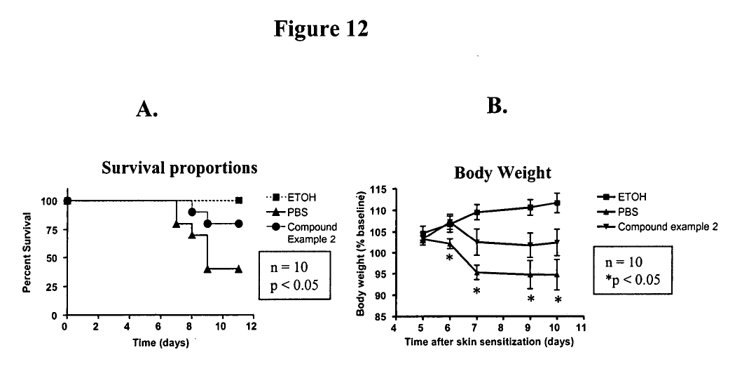

To investigate further the role of SSAO inhibition in the inflammatory response, studies were carried out to assess the effect of SSAO inhibition on prostaglandin E₂ (PGE2) levels. Animals were divided into four therapeutic groups of eight rats each. Three groups received oral administration of either 50 mg/kg of the compound of example 2; 3 mg/kg indomethacin; or PBS, respectively, 1 hour prior to carrageenan injections. The fourth group received 3 mg/kg of dexamethasone, i.p. 1 hour before paw inflammation. Three hours after carrageenan injection, rats were asphyxiated with CO₂ and their hind paws removed. The paws were lacerated with a scalpel, suspended off the bottom of a polypropylene 1.5 ml tube with a micropipette tip and centrifuged to express the inflammatory fluid. The volume collected from each paw was determined and the fluid was analyzed by ELISA for PGE2 production using a commercial kit (R&D Systems, Minneapolis, Minn.) according to the manufacturer's instructions. Carrageenan injection into footpad typically induces a 5- to 10-fold increase in PGs. As expected, dexamethasone was more effective at preventing swelling, whereas indomethacin had a greater impact on PGE2 levels (see FIG. 11). The compound of example 2 was able to significantly reduce PGE2 production to levels equivalent to those observed in the dexamethasone-treated animals. Data were analyzed with GraphPad Prism software (San Diego, Calif.) by Dunnett's test following analysis of variance.

### Example 20

### Inhibition of Chemically-Induced Colitis

2,4,6-trinitrobenzene sulfonic acid (TNBS)-induced colitis and dextran sodium sulfate (DSS)-induced colitis are TH1-mediated mouse models of colitis related to Crohn's disease. Compounds acting through various mechanisms have been demonstrated to be effective in these models, including prednisolone, anti IL-16, anti-ICAM, and anti-integrin, among many others (Strober W. et al (2002) Annu. Rev. Immunol. 20: 495). Oxazolone-induced colitis is a TH2-mediated process that closely resembles ulcerative colitis and is responsive to anti-IL4 therapy (Boirivant M. et al. (1998) J. Ex. Med 188: 1929).

TNBS colitis is induced as described (Fuss I. J. et al. (2002) J. Immunol: 168: 900). Briefly, 2.5 mg/mouse of TNBS (pH 1.5-2, Sigma) in 50% ETOH is administered intrarectally in anesthetized SJL/J male mice through a 3.5 F catheter inserted 4 cm proximal to the anal verge. TNBS-injected mice are divided in three treatment groups and injected i.p. twice a day with: PBS; prednisolone (5 mg/kg) and a compound of the invention (at, e.g., 20 mg/kg). Injections are initiated at day 0 (day of TNBS injection) and are continued through day 7.

Oxazolone colitis is induced as described (Fuss I. J. et al. (2002) J. Immunol. 168: 900). Briefly, mice are pre-sensitized by skin epicutaneous application of 3% oxazolone (4-ethoxymethylene-2-phenyl-2oxazolin-5-one, Sigma) in 100% EtOH (150 μl) on day 0, followed by intrarectal administration of 1% oxazolone in 50% EtOH (100 μl) to anesthetized SJL/J male mice on day 5 through a 3.5 F catheter inserted 4 cm proximal to the anal verge. Mice are divided in three treatment groups and injected i.p. twice a day with: PBS and a compound of the invention. Injections are initiated at day 0 and are continued through day 7 or the end of the study.

Colitis is also induced by feeding Balb/c mice with 5% (wt/vol) DSS (ICN Biomedicals Inc., Ohio, USA) for 7 days as described (Okayasu I. et al. (1990) Gastroenterology 98: 694). Mice are divided in three treatment groups and injected i.p. twice a day with: PBS, prednisolone (5 mg/kg) and a compound of the invenvion (at, e.g., 20 mg/kg). Injections are initiated at day 0 (first day of DSS feeding) and are continued through day 7.

Disease progression is evaluated in all models by monitoring body weight, stool consistency, presence of blood in stool, histologic analysis of colon tissues sections, and monitoring levels of several cytokines.

### Example 20A

### Inhibition of Oxazolone-Induced Colitis

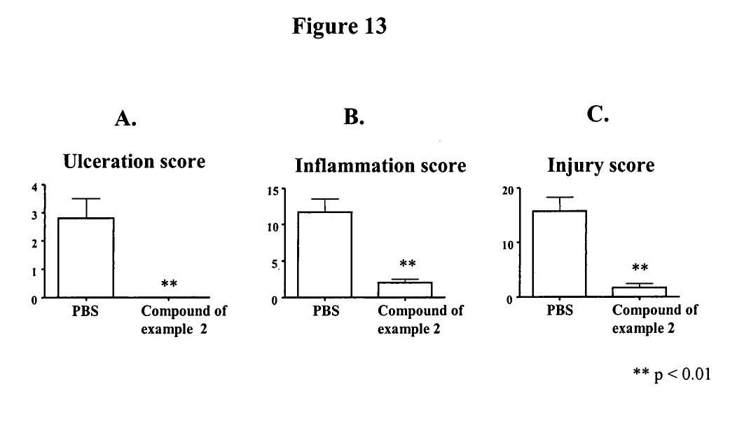

A study was carried out using the protocol in Example 20 for oxazolone-induced colitis. Injections were initiated at day 0 and were continued through the end of the study. Disease progression was evaluated for 12 days (6 days after intrarectal administration) by monitoring survival rates and body weight, as well as by macroscopic evidence of colitis (i.e. rectal prolapse, colon size, colon weight). (2-phenylallyl)hydrazine was administered at 10 mg/kg, i.p. twice a day, starting on the day of skin pre-sensitization (day 0). Results showed that (2-phenylallyl)hydrazine significantly improved survival rates and body weight loss when compared with the vehicle group (see FIGS. 12A and 12B). Kaplan-Meyer survival curves and unpaired t tests were calculated using GraphPad Prism software (San Diego, Calif.).

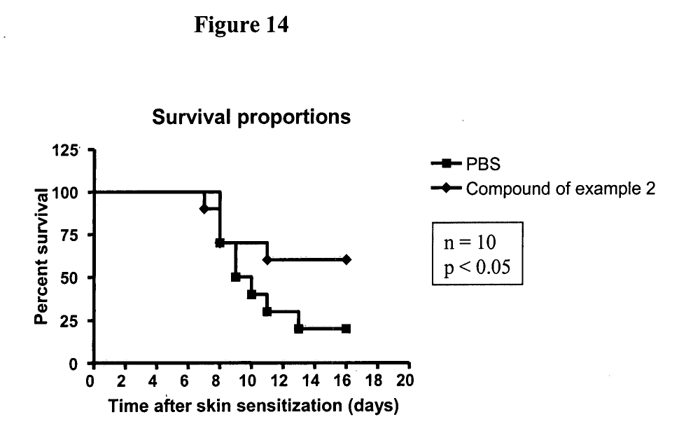

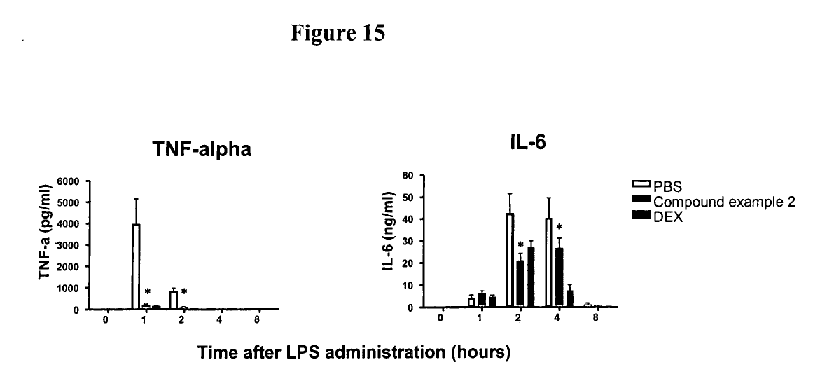

When following the above-described protocol, disease severity as measured by body weight drop was maximal at day 7 (2 days after intrarectal challenge), which is also the day when animals start dying. Thus, animals from a similar study were sacrificed seven days after initial sensitization and their colons removed and fixed in 1% formalin. Tissue processing and analysis were performed blindly at a contract laboratory (Pathology Associates, Frederick, Md.). Briefly, after paraffin embedding 5 μm sections were cut and stained with hematoxylin and eosin. Three cross sections were taken from each animal at 1 cm (section 1), 3 cm (section 2), and 6 cm (section 3) from the anus. The degrees of ulceration, inflammation (in mucosa, submucosa, serosa, and outer muscular layers) and epithelial injury (including mucosal and submucosal abscess, submucosal fibrosis, glandular distortion and mucosal and submucosal edema/hemorrhage), were graded semiquantitatively as 0—absent; 1—minimal; 2—mild; 3—moderate; 4—marked. FIG. 13 shows that (2-phenylallyl)hydrazine had a significant effect on the ulceration, inflammatory and injury indexes (see FIG. 13A, FIG. 13B, and FIG. 13C, respectively). In another study dosing was started one day after intrarectal challenge (day 6) to determine whether administration of SSAO inhibitor after disease induction would have any impact on survival. Data of one representative experiment is shown in FIG. 14. Administration of (2-phenylallyl)hydrazine after disease onset had a significant impact on survival. Kaplan-Meyer survival curves were calculated using GraphPad Prism software (San Diego, Calif.).

### Example 21

### Inhibition of Concanavalin A-Induced Liver Injury

Prevention of inflammation by administration of compounds of the invention is assessed in the concanavalin A (Con A) murine model of liver injury. Con A activates T lymphocytes and causes T cell-mediated hepatic injury in mice. Tumor necrosis factor alpha is a critical mediator in this experimental model. T-cell-mediated liver injury involves the migration of immune cells, notably CD4+ T lymphocytes, into liver tissue. Balb/c mice are inoculated with 10 mg/kg concanavalin A administered i.v. in 200 μl pyrogen-free saline as described (Willuweit A. et al. (2001) J. Immunol. 167:3944). Previous to Con A administration, animals are separated into treatment groups and injected i.p with: PBS, and different concentrations of compound of the invention (e.g., 20 mg/kg). Liver damage is evaluated by determining serum levels of liver enzymes such as transaminase and alkaline phosphatase, hepatic histopathology, and levels of of different inflammatory cytokines in plasma and liver tissue.

### Example 22

### Inhibition of Cutaneous Inflammation in the SCID Mouse Model of Psoriasis

Recent establishment of the SCID-human skin chimeras with transplanted psoriasis plaques has opened new vistas to study the molecular complexities involved in psoriasis. This model also offers a unique opportunity to investigate various key biological events such as cell proliferation, homing in of T cells in target tissues, inflammation and cytokine/chemokine cascades involved in an inflammatory reaction. The SCID mouse model has been used to evaluate the efficacy of several compounds for psoriasis and other inflammatory diseases (Boehncke W. H. et al. (1999) Arch Dermatol Res. 291(2-3):104).

Transplantations are done as described previously (Boehncke, W. H. et al. (1994) Arch. Dermatol. Res. 286:325). Human full-thickness xenografts are transplanted onto the backs of 6- to 8-week-old C.B17 SCID mice (Charles River). For the surgical procedure, mice are anesthetized by intraperitoneal injection of 100 mg/kg ketamine and 5 mg/kg xylazine. Spindle-shaped pieces of full-thickness skin measuring 1 cm in diameter are grafted onto corresponding excisional full-thickness defects of the shaved central dorsum of the mice and fixed by 6-0 atraumatic monofilament sutures. After applying a sterile petroleum jelly-impregnated gauze, the grafts are protected from injury by suturing a skin pouch over the transplanted area using the adjacent lateral skin. The sutures and over-tied pouches are left in place until they resolve spontaneously after 2-3 weeks. Grafts are allowed 2 weeks for acceptance and healing. Thereafter, daily intraperitoneal injections are performed between days 15 and 42 after transplantation. Mice are injected with either vehicle (PBS), dexamethasone (0.2 mg/kg body weight), or a compound of the invention (at, e.g., 20 mg/kg body weight) in a final volume of 200 μl. Mice are sacrificed at day 42, and after excision with surrounding mouse skin the grafts are formalin-embedded. Subsequently, routine hematoxylin-and-eosin staining is performed, and the grafts are analyzed with regard to their pathological changes both qualitatively (epidermal differentiation, inflammatory infiltrate) and quantitatively (epidermal thickness).

### Example 23

### Effect of Compounds of the Invention in a Mouse Model of Alzheimer 's Disease

Alzheimer's disease (AD) is characterized clinically by a dementia of insidious onset and pathologically by the presence of numerous neuritic plaques and neurofibrillary tangles. The plaques are composed mainly of β-amyloid (Aβ) peptide fragments, derived from processing of the amyloid precursor protein (APP). Tangles consist of paired helical filaments composed of the microtubule-associated protein, tau. Transgenic mice carrying a pathogenic mutation in APP show marked elevation of Aβ-protein level and Aβ deposition in the cerebral cortex and hippocampus from approximately 1 year of age (Hsiao K. et al. (1996) Science 274:99). Mutant PS-1 transgenic mice do not show abnormal pathological changes, but do show subtly elevated levels of the Aβ42/43 peptide (Duff K, et al. (1996) Nature 383:710). Transgenic mice derived from a cross between these mice (PS/APP) show markedly accelerated accumulation of Aβ into visible deposits compared with APP singly transgenic mice (Holcomb L. et al. (1998) Nat Med 4:97). Further, a recent study indicates that in these mice, inflammatory responses may be involved in the Aβ depositions (Matsuoka Y. et al. (2001) Am J Pathol. 158(4):1345).

The PS/APP mouse, therefore, has considerable utility in the study of the amyloid phenotype of AD and is used in studies to assess efficacy of the compounds of the invention to treat Alzheimer's patients. Mice are injected with vehicle (e.g., PBS) or a compound of the invention (at, e.g., 10-20 mg/kg), and are evaluated by analysis of memory deficits, histological characteristics of sample tissues, and other indicators of disease progression.

### Example 24

### Effect of Compounds of the Invention in Murine Models of Type I Diabetes Mellitus

It is widely accepted that proinflammatory cytokines play an important role in the development of type 1 diabetes. Thus, compounds of the invention can be used to treat patients suffering from this disease. A mouse with diabetes induced by multiple low doses of streptozotocin (STZ) can be used as an animal model for type 1 diabetes. STZ is used to induce diabetes in C57BL/6J mice. Briefly, STZ (40 mg/kg) or citrate buffer (vehicle) is given i.p. once daily for 5 consecutive days as described (Carlsson P. Oet al. (2000) Endocrinology. 141(8):2752). Compound administration (i.p. 10 mg/kg, twice a day) are started 5 days before STZ injections and continue for 2 weeks. Another widely use model is the NOD mouse model of autoimmune type 1 diabetes (Wong F. S. and Janeway C. A. Jr. (1999) Curr Opin Immunol. 11 (6):643. Female NOD mice are treated with daily injections of a compound of the invention (20 mg/kg/day) from week 10 through week 25. The effect of the compounds of the invention in preventing the development of insulitis and diabetes in NOD-scid/scid females after adoptive transfer of splenocytes from diabetic NOD females is also assessed. For both the STZ and NOD models, the incidence of diabetes is monitored in several ways, including monitoring of blood glucose levels. Insulin secretion is assessed in pancreatic islets isolated from experimental mice. Cytokine production is measured in mouse sera. Islet apoptosis is assessed quantitatively.

### Example 25

### Effect of Compounds of the Invention in Models of Airway Inflammation

Anti-inflammatory compounds such as SSAO inhibitors can have beneficial effects in airway inflammatory conditions such as asthma and chronic obstructive pulmonary disease. The rodent model here described has been extensively used in efficacy studies. Other murine models of acute lung inflammation can also be used to test the compounds of the invention.

For the evaluation of the effects of SSAO inhibitors in preventing airway inflammation, three groups of sensitized rats are studied. Animals are challenged with aerosolized OVA (ovalbumin) after intraperitoneal administration of the vehicle saline, a compound of the invention, or a positive control (e.g. prednisone) twice daily for a period of seven days. At the end of the week animals are anesthetized for measurements of allergen-induced airway responses as described (Martin J. G. et al. (2002) J. Immunol. 169(7):3963). Animals are intubated endotracheally with polyethylene tubing and placed on a heating pad to maintain a rectal temperature of 36° C. Airflow is measured by placing the tip of the endotracheal tube inside a Plexiglas box (˜250 ml). A pneumotachograph coupled to a differential transducer is connected to the other end of the box to measure airflow. Animals are challenged for 5 min with an aerosol of OVA (5% w/v). A disposable nebulizer will be used with an output of 0.15 ml/min. Airflow is measured every 5 min for 30 min after challenge and subsequently at 15-min intervals for a total period of 8 h. Animals are then sacrificed for bronchoalveolar lavage (BAL). BAL is performed 8 h after challenge with five instillations of 5 ml of saline. The total cell count and cell viability is estimated using a hemacytometer and trypan blue stain. Slides are prepared using a Cytospin and the differential cell count is assessed with May-Griinwald-Giemsa staining, and eosinophil counts by immunocytochemistry.

### Example 26

### Oral Bioavailability Studies in Rodents

Oral bioavailability studies in mice and rats were performed. Briefly, C57B1/6 female mice and Sprague Dawley female rats were administered 50 mg/kg of different compounds of the invention by oral gavage. Animals were bled at different time intervals after compound administration and the levels of inhibitor in plasma were determined using the colorimetric assay described in Example 14. Results of representative experiments are shown in FIG. 5 and indicated that the compounds of the invention are orally bioavailable. (FIG. 5A shows results in mice; FIG. 5B shows results in rats.) Thus, these data indicate that the small molecule SSAO inhibitors herein described allow for the development of an orally administered drug. The same studies were carried out after intravenous and intraperitoneal administration of different doses of the compounds of the invention described in examples 2, 8 and 10. The results show that these compounds were also readily bioavailable after those forms of administration.

### Example 27

### Dose-Response Effect After In Vivo Administration of SSAO/VAP-1 Inhibitors

In vivo inhibition of SSAO was assessed in rat aorta and lungs, two of the tissues where SSAO activity is highest. Six week old female Sprague Dawley rats were administered 0, 0.1, 1, 10 and 50 mg/kg of the compound of example 8 in 2.5 ml/kg PBS by oral gavage. Four hours after compound administration the animals were euthanized and their aortas and lungs removed and frozen in liquid nitrogen. Tissues were homogenized in 0.1 M potassium phosphate pH 7.8 buffer (30 ml/g for aorta and 20 ml/g for lung) and centrifuged at 1000×g for 15 min. Supernatants were collected and used in the radioactive assay following the protocol described by Lizcano J. M. et al. (1998) Biochem. J. 331:69. Enzymatic reactions were initiated by incubating a 200 μl aliquot of the tissue homogenate with 20 μl of 0.4 mM ¹⁴C-labeled benzylamine substrate (6 mCi/mmol specific activity, Pharmacia) for 30 min at RT. The assay was stopped by addition of 100 μl of 2 M citric acid, the assay volume was extracted with 5 ml toluene:ethyl acetate (1:1) containing 0.6% (w/v) 2,5-diphenyloxdazole (PPO), and an aliquot of the organic layer was counted by liquid scintillation. Because SSAO and MAO-B are both active towards benzylamine, control samples needed to be run concomitantly so that MAO-B and SSAO activities could be identified. SSAO was inhibited with 0, 10, 50 and 500 μM of semicarbazide for MAO-B determinations, and MAO-B was inhibited with 0, 5, and 100 μM of pargyline for SSAO determinations. These inhibitors were added to the tissue supernatant prior to addition of benzylamine. Aorta and lung had mainly SSAO activity; these results are in accordance with published data. FIG. 6 shows that in vivo ED₅₀ values for the compound of example 8 were 0.72 mg/kg and 5 mg/kg for lung and aorta, respectively.

### Example 28

### Blocking of In Vitro Adhesion by SSAO/VAP-1 Inhibitors

The studies in this Example were carried out in order to determine whether SSAO/VAP-1 transfected into endothelial cells retained the adhesion function and whether it played any role in the adhesion of freshly isolated human PBMCs to these cells. Moreover, the studies were also designed to determine whether blocking of SSAO/VAP-1 would have an impact on the level of adhesion between these two cell types. Adhesion assays were performed using cells labeled with the fluorescent dye Calcein-AM (Molecular Probes, OR, USA) as per the manufacturer's instructions. Briefly, rat lymph node high endothelial cells (HEC; isolation and culture was described in Ager, A. (1987) J. Cell Sci. 87: 133) were plated overnight in 96-well plates (2,000 cells/well). PBMCs (peripheral blood mononuclear cells) (1×10⁷) were labeled with 1 ml of 10 μM Calcein-AM for 1 hr at 37° C., washed three times with RPMI, and added to the 96 well plates containing monolayers of HEC cells mock-transfected or transfected with full-length human SSAO/VAP-1 (60,000 PBMCs were plated per well containing 2,000 HEC cells). Adhesion was carried out for 3 hr at 37° C. Non-adherent cells were removed by washing three times with RPMI and fluorescence was measured in a fluorescence plate reader at an excitation wavelength of 485 nm and emission wavelength of 530 nm. Several controls were included, such as HEC cells and PBMCs (labeled and unlabeled) alone. In all experiments, SSAO/VAP-1 expression increased adhesion of PBMCs to HEC cells by 2-5 fold. These results are in agreement with data published by others (Smith et al. J Exp Med (1998) 188:17; Salmi et al. Circ Res (2000) 86:1245).

The next experiments were designed in order to investigate whether blocking the enzymatic catalytic site has any effect on the adhesion function of SSAO/VAP-1, and whether or not inhibitors according to the invention could mediate an adhesion-inhibiting effect. Published results suggest that blocking SSAO enzymatic activity with semicarbazide inhibited lymphocyte rolling under laminar sheer on cardiac endothelial monolayers (Salmi et al. Immunity (2001) 14:265). These studies were repeated using the adhesion assay as described above to evaluate the inhibitors of the invention. Adhesion blockers used included an anti-human VAP-1 monoclonal antibody (Serotec, Oxford, UK), neuramidase (a sialidase, because SSAO/VAP-1 is a sialoglycoprotein; Sigma), and several function-blocking antibodies to rat adhesion molecules (CD31-PECAM, CD54-ICAM-1, CD92P-P Selectin). Controls included the SSAO inhibitor semicarbazide (Sigma), MAO-A and MAO-B inhibitors (clorgyline and pargyline, respectively; Sigma), and mouse IgG1 and IgG2 isotype controls (BD, USA). Antibodies (10 μg/ml) and neuramidase (5 mU) were incubated with the HECs for 30 min at 37° C.; excess antibody was washed away prior to the addition of the labeled PBMCs. Small-molecule inhibitors were pre-incubated the same way at IC₁₀₀ concentrations, but the amounts present in the supernatant were not washed away to preserve the IC₁₀₀ concentration during the adhesion step.

FIG. 7 shows the results of one representative experiment (n=6 replicates). FIG. 7B shows data indicating that anti-VAP-1, the compound of example 2, the compound of example 8, and, to a lesser extent, semicarbazide reduced the number of PBMCs adherent to SSAO/VAP-1 transfected HECs to levels close to the ones observed in the mock-transfected cells. (The data for the same compounds tested against mock-transfected cells is depicted in FIG. 7A.) The anti-VAP-1 antibody results here are in agreement with the published data regarding the effect of anti-VAP-1 mAb on the adhesion of lymphocytes to VAP-1-transfected HEC cells (Salmi et al Circ Res (2000) 86:1245). Clorgyline (MAO-A inhibitor) and pargyline (MAO-B inhibitor) had no effect. Interestingly, VAP-1 expression seems to decrease the relative blocking effect of anti-CD54, CD31 and CD62P antibodies.

In summary, these results indicate that the compounds of the invention which inhibit SSAO enzymatic function reduced binding of PBMCs to SSAO/VAP-1-expressing HECs in vitro; that is, the SSAO inhibitors of the invention were able to inhibit adhesion of PBMCs to SSAO/VAP-1-expressing HECs in vitro.

### Example 29

### Inhibition of Lipopolysaccharide (LPS)-Induced Endotoxemia

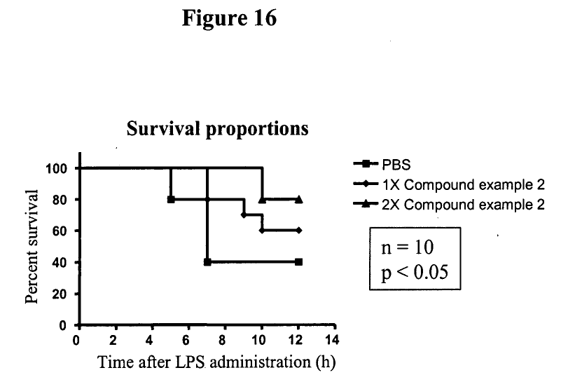

In sepsis exposure of endothelial cells of all organs to elevated levels of LPS and inflammatory cytokines leads to upregulation of adhesion molecules and chemokines, which results in an increase in the tethering, rolling and transmigration of leukocytes (Pawlinski R. et al. (2004) Blood 103:1342). LPS-induced endotoxemia is a well-characterized model of systemic inflammation and thus can be used to investigate the putative role of SSAO inhibition in these inflammatory mechanisms. Sepsis was induced in C57B1/6J female mice by i.p. administration of 5 mg/kg of LPS. Sixty minutes prior to LPS injections, 200 μl of vehicle (PBS) or 50 mg/kg of (2-phenylallyl)hydrazine was administered orally to the animals. Dexamethasone was administered i.p, at a concentration of 3 mg/kg 1 hr prior to disease induction. Blood was drawn from the retroorbital plexus of anesthetized animals and sera was collected and frozen until time of cytokine measurements. IL-1β, TNF-α, and IL-6 concentrations were determined by ELISA using commercial kits (R&D Systems, Minneapolis, Minn.) according to the manufacturer's instructions. FIG. 15 shows that (2-phenylallyl)hydrazine significantly reduced levels of circulatory TNF-α and IL-6 in this model. Data were analyzed with GraphPad Prism software (San Diego, Calif.) by Dunnett's test following analysis of variance. FIG. 16 shows the results of a study designed to investigate whether SSAO inhibition is able to affect survival of animals after LPS shock. 2 mg/kg LPS together with 300 mg/kg D-galactosamine (GalN, Sigma), both dissolved in PBS, were administered to mice by i.p. injection. At the indicated time points animals from the different therapeutic groups received 200 μl of vehicle (PBS) or 30 mg/kg (2-phenylallyl)hydrazine by oral administration. Data indicate that SSAO inhibition prolongs the survival of mice post LPS shock.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A compound of formula I-P: 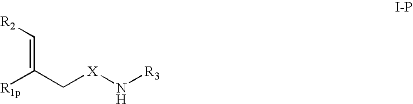
wherein R₁ₚ is independently chosen from the group consisting of H, C₁-C₄ alkyl,
C₃-C₈ cycloalkyl, C₆-C₁₀ aryl, C₆-C₁₄ aralkyl, C₄-C₉ heteroaryl, C₆-C₁₄ substituted aryl,
C₅-C₁₄ substituted heteroaryl, R₄—(CH₂)ₙ—, and R₅—Y₁—CH₂—;
n is independently 1 or 2;
Y₁ is independently S or O;
R₂ is independently chosen from H, C₁-C₄ alkyl, Cl, F, or CF₃;
X is independently chosen from O or NR₆;
R₃ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₄ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₅ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl; and
R₆ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
with the proviso that when R₁ is unsubstituted phenyl, R₂ is H, and X is NH, then R₃ is not H;
including all stereoisomers thereof, all E/Z (cis/trans) isomers thereof, all solvates and hydrates thereof, all crystalline and non-crystalline forms thereof, and all salts thereof

2. A compound of claim 1, where R₁ₚ is unsubstituted phenyl.

3. A compound of claim 1, where R₁ₚ is substituted phenyl.

4. A compound of claim 1, where R₂ is H.

5. A compound of claim 1, where X is O.

6. A compound of claim 1, where X is NR₆.

7. A compound of claim 1, where R₃ is H or C₁-C₄ alkyl.

8. A compound of claim 1, where R₆ is H or C₁-C₄ alkyl.

9. A compound of claim 1 according to formula I-AP: 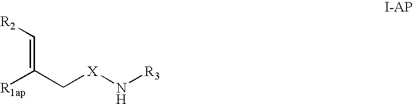
wherein:
R₁ₐₚ is substituted or unsubstituted phenyl;
R₂ is independently chosen from H, C₁-C₄ alkyl, Cl, F, or CF₃;
X is independently chosen from O or NR₆;
R₃ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₆ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
with the proviso that when R₁ is unsubstituted phenyl, R₂ is H, and X is NH, then R₃ is not H;
including all stereoisomers thereof, all E/Z (cis/trans) isomers thereof, all solvates and hydrates thereof, all crystalline and non-crystalline forms thereof, and all salts thereof.

10. A compound of claim 9, where R₁ₐₚ is unsubstituted phenyl.

11. A compound of claim 9, where R₁ₐₚ is substituted phenyl.

12. A compound of claim 9, where X is O.

13. A compound of claim 9, where X is NR₆.

14. A compound of claim 9, where R₃ is H or C₁-C₄ alkyl.

15. A compound of claim 9, where R₆ is H or C₁-C₄ alkyl.

16. A compound of formula I-B: 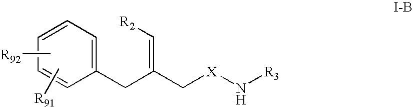
wherein:
R₂ is independently chosen from H, C₁-C₄ alkyl, Cl, F, or CF₃;
R₉₁ and R₉₂ are independently chosen from H, F, Br, Cl, I, C₁-C₄ alkyl, and C₁-C₄ alkoxy;
X is independently chosen from O or NR₆;
R₃ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₆ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
including all stereoisomers thereof, all E/Z (cis/trans) isomers thereof, all solvates and hydrates thereof, all crystalline and non-crystalline forms thereof, and all salts thereof.

17. A compound of claim 16, where X is NR₆.

18. A compound of claim 16, where R₃ is H or C₁-C₄ alkyl

19. A compound of claim 16, where R₆ is H or C₁-C₄ alkyl.

20. A compound of claim 16, where R₉₁ and R₉₂ are both H.

21. A compound of formula I-C: 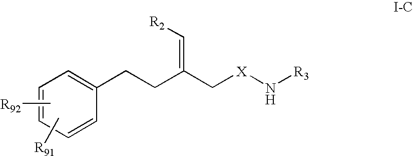
wherein:
R₂ is independently chosen from H, C₁-C₄ alkyl, Cl, F, or CF₃;
R₉₁ and R₉₂ are independently chosen from H, F, Br, Cl, I, C₁-C₄ alkyl, and C₁-C₄ alkoxy;
X is independently chosen from O or NR₆;
R₃ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
R₆ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
including all stereoisomers thereof, all E/Z (cis/trans) isomers thereof, all solvates and hydrates thereof, all crystalline and non-crystalline forms thereof, and all salts thereof.

22. A compound of claim 21, where X is NR₆.

23. A compound of claim 21, where R₃ is H or C₁-C₄ alkyl

24. A compound of claim 21, where R₆ is H or C₁-C₄ alkyl.

25. A compound of claim 21, where R₉₁ and R₉₂ are both H.

26. A compound of formula III: 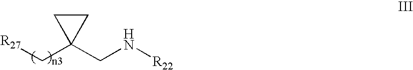
wherein R₂₇ is independently chosen from H, C₁-C₄ alkyl, C₃-C₈ cycloalkyl, C₆-C₁₀ aryl,
C₆-C₁₄ aralkyl, C₄-C₉ heteroaryl, C₆-C₁₄ substituted aryl, C₅-C₁₄ substituted heteroaryl,
R₂₃—(CH₂)ₙ—, and R₂₄—Y₂—(CH₂)—;
R₂₂ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
n is independently 1 or 2;
n3 is independently 0, 1, or 2;
Y₂ is independently S or O; and
R₂₃ and R₂₄ are independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
including all stereoisomers thereof, all E/Z (cis/trans) isomers thereof, all solvates and hydrates thereof, all crystalline and non-crystalline forms thereof, and all salts thereof.

27. A compound of claim 26, wherein R₂₇ is unsubstituted phenyl.

28. A compound of claim 26, wherein R₂₇ is substituted phenyl.

29. A compound of claim 26, wherein R₂₂ is H or C₁-C₄ alkyl.

30. A compound of claim 26 of the formula III-A: 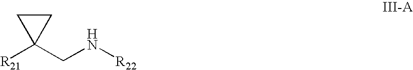
wherein R₂₁ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl, C₅-C₁₄ substituted heteroaryl, R₂₃—(CH₂)ₙ—, and R₂₄—Y₂—(CH₂)—;
R₂₂ is independently chosen from H, C₁-C₄ alkyl, C₃-Cg cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
n is independently 1 or 2;
Y₂ is independently S or O; and
R₂₃ and R₂₄ are independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
including all stereoisomers thereof, all E/Z (cis/trans) isomers thereof, all solvates and hydrates thereof, all crystalline and non-crystalline forms thereof, and all salts thereof.

31. A compound of claim 30, wherein R₂₇ is unsubstituted phenyl.

32. A compound of claim 30, wherein R₂₇ is substituted phenyl.

33. A compound of claim 30, wherein R₂₂ is H or C₁-C₄ alkyl.

34. A compound of claim 26 of the formula III-B: 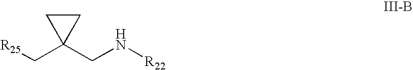
wherein R₂₅ is independently chosen from C₆-C₁₀ aryl, C₆-C₁₄ aralkyl, C₄-C₉ heteroaryl, C₆-C₁₄ substituted aryl, and C₅-C₁₄ substituted heteroaryl; and
R₂₂ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
including all stereoisomers thereof, all E/Z (cis/trans) isomers thereof, all solvates and hydrates thereof, all crystalline and non-crystalline forms thereof, and all salts thereof.

35. A compound of claim 34, wherein R₂₇ is unsubstituted phenyl.

36. A compound of claim 34, wherein R₂₇ is substituted phenyl.

37. A compound of claim 34, wherein R₂₂ is H or C₁-C₄ alkyl.

38. A compound of formula III-C: 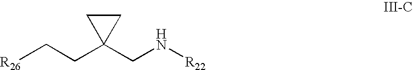
wherein R₂₆ is independently chosen from C₆-C₁₀ aryl, C₆-C₁₄ aralkyl, C₄-C₉ heteroaryl, C₆-C₁₄ substituted aryl, and C₅-C₁₄ substituted heteroaryl; and
R₂₂ is independently chosen from H, C₁-C₄ alkyl, C₃-C₈ cycloalkyl, C₆-C₁₀ aryl, C₆-C₁₄ aralkyl, C₄-C₉ heteroaryl, C₆-C₁₄ substituted aryl and C₅-C₁₄ substituted heteroaryl;
including all stereoisomers thereof, all E/Z (cis/trans) isomers thereof, all solvates and hydrates thereof, all crystalline and non-crystalline forms thereof, and all salts thereof.

39. A compound of claim 38, wherein R₂₇ is unsubstituted phenyl.

40. A compound of claim 38, wherein R₂₇ is substituted phenyl.

41. A compound of claim 38, wherein R₂₂ is H or C₁-C₄ alkyl.

42. A method of treating inflammation or an inflammatory disease in a mammal, comprising administering a compound of claim 1 in a therapeutically effective amount.

43. A method of treating an immune or autoimmune disease in a mammal, comprising administering a compound of claim 1 in a therapeutically effective amount.

44. A method of treating multiple sclerosis or chronic multiple sclerosis in a mammal, comprising administering a compound of claim 1 in a therapeutically effective amount.

45. A method of treating inflammation or an inflammatory disease in a mammal, comprising administering a compound of claim 9 in a therapeutically effective amount.

46. A method of treating an immune or autoimmune disease in a mammal, comprising administering a compound of claim 9 in a therapeutically effective amount.

47. A method of treating multiple sclerosis or chronic multiple sclerosis in a mammal, comprising administering a compound of claim 9 in a therapeutically effective amount.

48. A method of treating inflammation or an inflammatory disease in a mammal, comprising administering a compound of claim 16 in a therapeutically effective amount.

49. A method of treating an immune or autoimmune disease in a mammal, comprising administering a compound of claim 16 in a therapeutically effective amount.

50. A method of treating multiple sclerosis or chronic multiple sclerosis in a mammal, comprising administering a compound of claim 16 in a therapeutically effective amount.

51. A method of treating inflammation or an inflammatory disease in a mammal, comprising administering a compound of claim 21 in a therapeutically effective amount.

52. A method of treating an immune or autoimmune disease in a mammal, comprising administering a compound of claim 21 in a therapeutically effective amount.

53. A method of treating multiple sclerosis or chronic multiple sclerosis in a mammal, comprising administering a compound of claim 21 in a therapeutically effective amount.

54. A method of treating inflammation or an inflammatory disease in a mammal, comprising administering a compound of claim 26 in a therapeutically effective amount.

55. A method of treating an immune or autoimmune disease in a mammal, comprising administering a compound of claim 26 in a therapeutically effective amount.

56. A method of treating multiple sclerosis or chronic multiple sclerosis in a mammal, comprising administering a compound of claim 26 in a therapeutically effective amount.

57. A method of treating inflammation or an inflammatory disease in a mammal, comprising administering a compound of claim 30 in a therapeutically effective amount.

58. A method of treating an immune or autoimmune disease in a mammal, comprising administering a compound of claim 30 in a therapeutically effective amount.

59. A method of treating multiple sclerosis or chronic multiple sclerosis in a mammal, comprising administering a compound of claim 30 in a therapeutically effective amount.

60. A method of treating inflammation or an inflammatory disease in a mammal, comprising administering a compound of claim 34 in a therapeutically effective amount.

61. A method of treating an immune or autoimmune disease in a mammal, comprising administering a compound of claim 34 in a therapeutically effective amount.

62. A method of treating multiple sclerosis or chronic multiple sclerosis in a mammal, comprising administering a compound of claim 34 in a therapeutically effective amount.

63. A method of treating inflammation or an inflammatory disease in a mammal, comprising administering a compound of claim 38 in a therapeutically effective amount.

64. A method of treating an immune or autoimmune disease in a mammal, comprising administering a compound of claim 38 in a therapeutically effective amount.

65. A method of treating multiple sclerosis or chronic multiple sclerosis in a mammal, comprising administering a compound of claim 38 in a therapeutically effective amount.
